(19) United States
(12) Patent Application Publication
(10) Pub. No.: US 2026/0234574 A1
(43) Pub. Date: Aug. 13, 2026

(54) BIOSYNTHESIS OF CANNABINOIDS FROM CANNABIGEROLIC ACID USING NOVEL CANNABINOID SYNTHASES

(71) Applicant: National University of Singapore, Singapore (SG)

(72) Inventors: Maybelle Darlene Kho Go, Singapore (SG); Wen Shan Yew, Singapore (SG)

(21) Appl. No.: 19/413,690

(22) Filed: Dec. 9, 2025

Related U.S. Application Data
(63) Continuation of application No. 17/767,764, filed on Oct. 12, 2020, now Pat. No. 12,497,600.
(60) Provisional application No. 62/913,991, filed on Oct. 11, 2019.

(51) Int. Cl. C12N 9/02; C12N 15/81; C12P 17/04; C12P 17/06; C12P 17/16; C12R 1/84; C12R 1/865

(52) U.S. Cl. CPC C12N 9/0004; C12N 15/815; C12P 17/04; C12P 17/06; C12P 17/162; C12R 2001/84; C12R 2001/865; C12Y 121/03007

(57) ABSTRACT

A method for producing a cannabinoid by contacting cannabigerolic acid with a cannabinoid synthase orthologue. The cannabinoid synthase orthologue is from an organism other than Cannabis sativa. Also disclosed is a recombinant cell of Saccharomyces cerevisiae or Pichia pastoris that includes in its genome a nucleic acid encoding the above cannabinoid synthase orthologue. The cannabinoid synthase orthologue is expressed in the recombinant cell in an active form.

### CROSS-REFERENCE TO RELATED APPLICATIONS

The present application is a continuation of U.S. application Ser. No. 17/767,764, filed Apr. 8, 2022, which is a National Phase Entry of International App. No. PCT/SG2020/050583, filed Oct. 12, 2020, which claims the benefit of and priority to U.S. Prov. App. Ser. No. 62/913,991, filed Oct. 11, 2019. The content of each of which is incorporated herein by reference in its entirety.

### SEQUENCE LISTING

The instant application contains a Sequence Listing which has been submitted electronically in ST.26 format and is hereby incorporated by reference in its entirety. The ST.26 copy, created Apr. 23, 2026, is named 218005-0108C1.xml and is 321,733 bytes in size.

### BACKGROUND

The cannabinoid biosynthetic pathway has been the subject of intense investigation. Cloning and expression of the various enzymes in the cannabinoid biosynthetic pathway has been accomplished by several research groups. For example, the structure and function of 41-tetrahydrocannabinolic acid synthase, i.e., a cannabinoid synthase, has been elucidated. See Shoyama et al., J. Mol. Biol. 2012, 423 (1): 96-105.

The biosynthesis of two cannabinoids, cannabidiolic acid and Δ⁹-tetrahydrocannabinolic acid, has been accomplished by expressing cannabinoid biosynthetic pathway enzymes from Cannabis sativa and other organisms heterologously in Saccharomyces cerevisiae. See, e.g., Luo et al., Nature 2019, 567 (7746): 123-126 and Zirpel et al., J. Biotechnol. 2017, 259:204-212. Studies such as these have established that it is possible to build the cannabinoid biosynthetic pathway in a heterologous system.

Additional enzymes and methods are needed for synthesizing cannabinoids with specificity and high yield.

### SUMMARY

A method is disclosed for producing a cannabinoid. The method includes the steps of contacting cannabigerolic acid with a cannabinoid synthase orthologue. The cannabinoid synthase orthologue is from an organism other than Cannabis sativa, e.g., Citrus sinensis, Cucumis melo, Capsicum annuum, Brassica napus, Nicotiana attenuata, Nicotiana tabacum, Noccaea caerulescens (Thlaspi caerulescens), Gossypium hirsutum (Gossypium mexicanum), Oryza sativa subsp. indica, Oryza sativa subsp. japonica, Arabidopsis lyrata subsp. lyrata, Paenibacillus sp. Aloe-11, Streptomyces ipomoeae 91-03, Brassica rapa subsp. pekinensis, Prunus persica, Bacillus subtilis 168, Arabidopsis thaliana, Papaver somniferum and Phytophthora 45 parasitica P1569.

Also provided are recombinant cells of Saccharomyces cerevisiae and Pichia pastoris that each include in their genomes a nucleic acid encoding a cannabinoid synthase orthologue, wherein the cannabinoid synthase orthologue is from any of the organisms listed in the preceding paragraph, and the cannabinoid synthase orthologue is expressed in the recombinant cells in an active form.

The details of one or more embodiments are set forth in the description and the examples below. Other features, objects, and advantages will be apparent from the detailed description, from the drawings, and also from the appended claims.

### BRIEF DESCRIPTION OF THE DRAWINGS

The invention description below refers to the accompanying drawings, of which:

FIG. 1 shows cannabinoid products that can be formed from cannabigerolic acid (“CBGA”) as substrate. The molecular formula and formula weight of cannabielsoic acid (“CBEA”) is C₂₂H₃₀O₅ and 374.5, respectively. The respective molecular formula and formula weight of cannabielsoin (decarboxylated CBEA) is C₂₁H₃₀O₃ and 330.5. The molecular formula and formula weight of all other compounds shown are C₂₂H₃₀O₄ and 358.5, respectively. CBCA=cannabichromenic acid, CBTA=cannabicitranic acid, CBDA=cannabidiolic acid, THICA=tetrahydroisocannabinolic acid, THCA=tetrahydrocannabinolic acid, and CBLA=cannabicyclolic acid.

FIG. 2A shows a representative total ion chromatogram of 18 reaction sets of cannabinoid synthase orthologues. Each reaction set took 1.7 min to analyze.

FIG. 2B shows a representative extracted ion chromatogram (“EIC”) of the 18 reaction sets of cannabinoid synthase orthologues obtained at mass to charge ratio “(m/z”)=359.5 showing the presence of CBGA that was added to 9 of the 18 reaction sets, i.e., sets 1-3, 7-9, 13-15.

FIG. 2C shows a representative EIC of the 18 reaction sets that exhibited the production of a cannabinoid having at m/z=357.5.

FIG. 3A shows a total-ion chromatograph of a group of 18 cannabinoid synthase orthologues. Each reaction set took 1.7 min.

FIG. 3B shows a representative EIC of the 18 reaction sets of cannabinoid synthase orthologues obtained at m/z=359.5 showing the presence of CBGA that was added to 9 of the 18 reaction sets, i.e., sets 1-3, 7-9, 13-15.

FIG. 3C shows a representative EIC of the 18 reaction sets that exhibited the production of a cannabinoid having at m/z=357.5.

FIG. 4. shows a representative chromatogram of a cannabinoid synthase orthologue that produced a cannabinoid having m/z=357.2071. Each reaction set 85 took 15.70 min. to analyze. Chromatograms in rows 1, 2, and 4 show the presence of a peak when CBGA was incubated with a positive cannabinoid synthase orthologue, the peak being 65-fold larger than the peak found in the control experiment (row 3) performed in the absence of a cannabinoid synthase orthologue.

FIG. 5 shows a representative chromatogram of a cannabinoid synthase 90 orthologue that produced a cannabinoid having m/z=373.2020. Each reaction set took 15.70 min. to analyze. Chromatograms in rows 2-4 show the presence of a peak when CBGA was incubated with a positive cannabinoid synthase orthologue, the peak being 120-fold larger than the peak found in the control experiment (row 1) performed in the absence of a cannabinoid synthase orthologue.

FIG. 6 shows a representative chromatogram of a cannabinoid synthase orthologue that produced a cannabinoid having m/z=329.2122. Each reaction set took 15.70 min. to analyze. Chromatograms in rows 1, 2, and 4 show the presence of a peak when CBGA was incubated with a positive cannabinoid synthase orthologue, the peak being 300-fold larger than the peak found in the control experiment (row 3) performed in the absence of a cannabinoid synthase orthologue.

### DETAILED DESCRIPTION

Disclosed are enzymes that catalyze the biosynthesis of cannabinoids from CBGA. These enzymes, not previously known as cannabinoid synthases, are from organisms other than Cannabis sativa. The enzymes can be recombinantly expressed in Saccharomyces cerevisiae and Pichia pastoris in an active form.

The method summarized above for producing a cannabinoid requires contacting CBGA with a cannabinoid synthase orthologue not from Cannabis sativa. The source of the cannabinoid synthase orthologue can be, but is not limited to, Citrus sinensis, Brassica napus, Nicotiana attenuate, Gossypium hirsutum, Oryza sativa subsp. indica, Oryza sativa subsp. japonica, Arabidopsis lyrata subsp. lyrata, Paenibacillus sp. Aloe-11, Streptomyces ipomoeae 91-03, Brassica rapa subsp. pekinensis, Prunus persica, Bacillus subtilis 168, Arabidopsis thaliana, Papaver somniferum, and Phytophthora parasitica P1569.

The cannabinoid synthase orthologue can be, but is not limited to, those shown 115 in Tables 1 and 2 below. Exemplary cannabinoid synthase orthologues can have the amino acid sequence of SEQ ID NOs: 7, 16, 23, 31, 37, 47, 57, 67, 77, 87, 97, 107, 117, and 127, or a sequence having 70% or greater (e.g., 70%, 75%, 80%, 85%, 90%, 95%, and 99%) identity to SEQ ID NOs: 7, 16, 23, 31, 37, 47, 57, 67, 77, 87, 97, 107, 117, and 127.

The method set forth above can produce cannabinoids having a formula weight of 358.5 g/mol, 374.5 g/mol, or 330.5 g/mol. In a particular method, cannabinoids having a formula weight of 358.5 g/mol, 374.5 g/mol, and 330.5 g/mol are each produced.

Another method produces cannabielsoic acid and cannabielsoin. An example of this method produces cannabinoids that include both cannabielsoic acid and cannabielsoin but the products are free of any cannabinoid having a formula weight of 358.5 g/mol. In this exemplary method, the cannabinoid synthase orthologue includes the amino acid sequence of SEQ ID NOs: 67, 77, 97, or 127 or a sequence having 70% or greater identity to SEQ ID NOs: 67, 77, 97, or 127.

In the methods described above, the cannabinoid synthase orthologue can be a recombinant enzyme. The recombinant enzyme can be produced in, e.g., Saccharomyces cerevisiae, Yarrowia lipolytica, Kluyveromyces marxianus, and Pichia pastoris. A specific method features a recombinant enzyme produced in Saccharomyces cerevisiae or Pichia pastoris.

Also mentioned above is a recombinant cell of Saccharomyces cerevisiae or Pichia pastoris contains in its genome a nucleic acid encoding a cannabinoid synthase orthologue. The orthologue is from an organism other than Cannabis sativa. Exemplary sources of the cannabinoid synthase orthologue are listed above and shown in Tables 1 and 2 below.

The recombinant cell can contain a nucleic acid that encodes a cannabinoid synthase orthologue that includes an amino acid sequence selected from the group consisting of SEQ ID NOs: 7, 16, 23, 31, 37, 47, 57, 67, 77, 87, 97, 107, 117, and 127, or a sequence having 70% or greater (e.g., 70%, 75%, 80%, 85%, 90%, 95%, and 99%) identity to SEQ ID NOs: 7, 16, 23, 31, 37, 47, 57, 67, 77, 87, 97, 107, 117, and 127.

Without further elaboration, it is believed that one skilled in the art can, based on the disclosure herein, utilize the present disclosure to its fullest extent. The following specific examples are, therefore, to be construed as merely descriptive, and not limitative of the remainder of the disclosure in any way whatsoever. All 150 publications cited herein are incorporated by reference in their entirety.

### EXAMPLES

### Example 1: Identification and Preparation of Potential Cannabinoid Synthases

Based on its sequence, THCA synthase can be classified as a berberine-bridge FAD-dependent enzyme. Through a search of available sequence databases, 232 related genes were identified that are annotated in the UniProt database as berberine-bridge FAD dependent enzymes. In other words, these 232 genes were potential cannabinoid synthase orthologues.

Each gene sequence was codon-optimized for S. cerevisiae protein expression, synthesized, and cloned into pYES2-CT vector (Thermo Fisher). The vectors were transformed separately into S. cerevisiae BY4741 cells using chemical treatment and grown on SC-URA-glucose selection plates for two days at 30° C. Three single colonies from each plate were picked and replicated onto SC-URA-glucose selection plates and incubated for two days at 30° C. The replicated colonies were grown in 10 mL SC-URA-glucose media for 16 h at 30° C. The cells were then harvested and resuspended in 5 mL SC-URA-galactose media and grown for protein expression for 16 h at 30° C. After 16 h, the cells were harvested again and stored at −20° C. Cells not carrying any cannabinoid synthase orthologue were also prepared as above to serve as a negative control.

The cells were resuspended in 400 μL of 100 mM citrate buffer, pH 5.5 with 1 mM MgCl₂ and 5 units of lyticase. The cells were incubated at 37° C. with shaking for 1 h. One gram of glass beads was added to the suspension and the cells were cracked open using the MP Biomedical FastPrep 24 Tissue Homogenizer. Cell debris was removed by centrifugation for 30 min. at 14,800 rpm at 4° C.

### Example 2: Enzymatic Assay

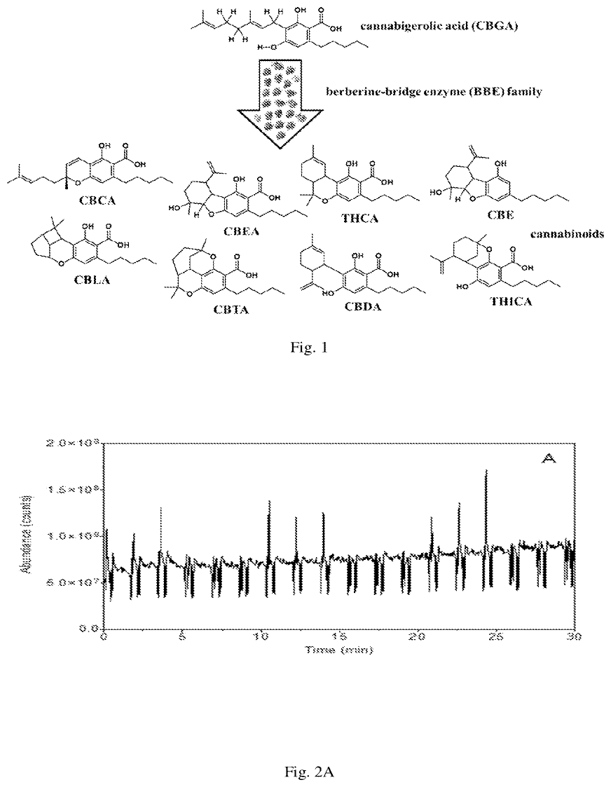

The potential cannabinoid synthase orthologues were tested for enzymatic activity as follows. In a reaction volume of 55 μL, 2.5 μL of 1 mg/mL CBGA, 2.5 μL of 20 mM FAD, and 50 μL of yeast cell supernatants prepared as above were combined and incubated under ambient conditions. A corresponding control reaction lacking CBGA was also carried out. After 24 h, the reactions were extracted three times with ethyl acetate. Samples of the extracted material were dried under vacuum and redissolved in acetonitrile for mass analysis using negative ion mode. EIC for m/z=357.5 and m/z=373.5 were generated for each sample to determine if the biosynthesis of a cannabinoid was catalyzed by the orthologues. The potential reaction products are shown in FIG. 1.

For each potential cannabinoid synthase orthologue, three single yeast colonies were picked and grown as mentioned above. The cell supernatant prepared from each colony was split into six portions. Three portions were incubated with CBGA and three portions were not incubated with CBGA as the negative control reaction. In total, for each potential orthologue, nine reactions were performed in the presence of CBGA and nine reactions were not incubated with CBGA.

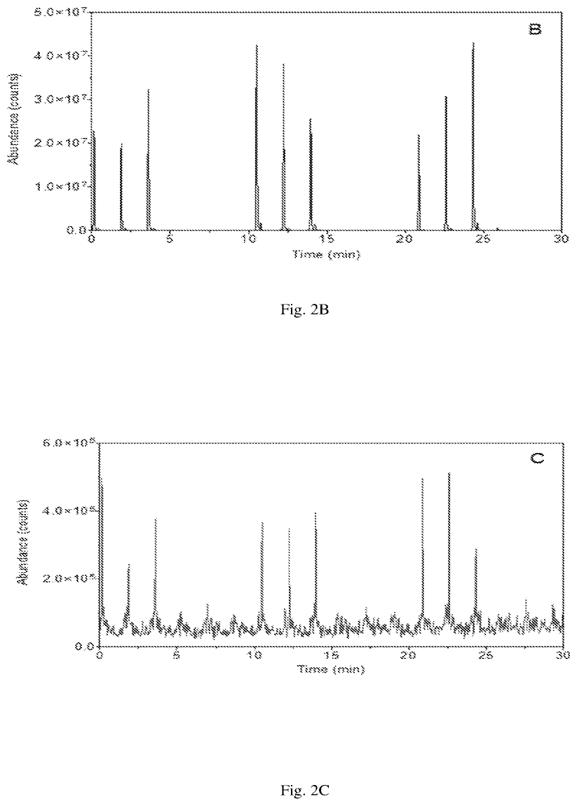

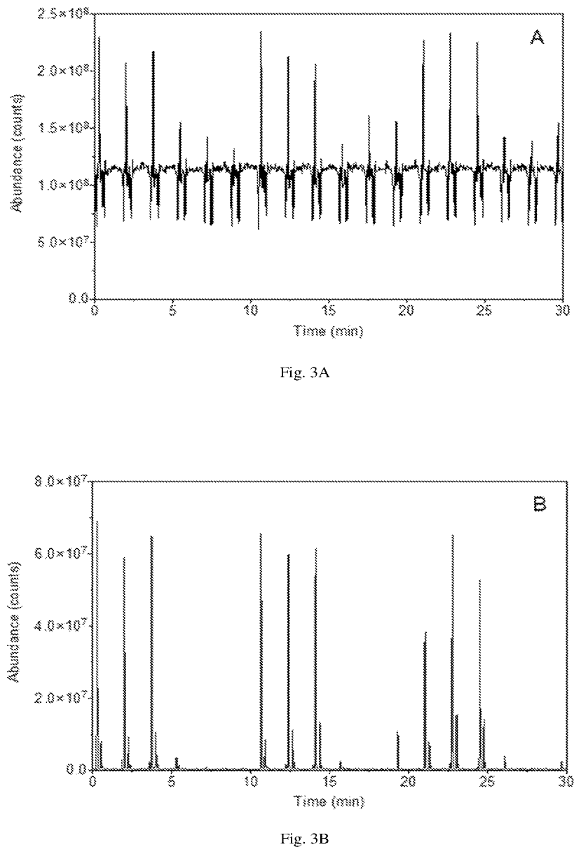

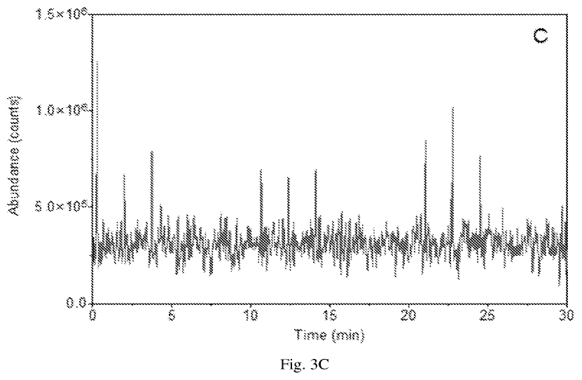

A sample incubated with CBGA was considered to be positive for cannabinoid synthesis if the extracted material had a chromatographic peak area in EIC having an abundance of at least 800,000 at m/z=357.5 and/or m/z=373.5. A potential cannabinoid synthase orthologue was considered to be positive if seven out of the nine total reactions with CBGA were positive using the above criteria. The results are shown in Table 1 below, as well as in FIGS. 2A-2C and FIGS. 3A-3C.

FIGS. 2A-2C and 3A-3C are representative chromatograms of a Rapid-Fire/Triple Quad mass analysis. FIG. 2C shows a representative chromatogram showing the presence of a peak at m/z 357.5. FIG. 3C shows a representative chromatogram comparing the presence of a peak at m/z 373.5.

TABLE 1
Activity of cannabinoid synthase orthologues
expressed in S. cerevisiae

| UniProt ID | | m/z = | m/z = |
|---|---|---|---|
| (SEQ ID NO) | Organism | 357.5 | 373.5 |
| A0A067ESH1 | Citrus sinensis | Yes | No |
| A0A078IY96 | Brassica napus | Yes | Yes |
| A0A1J6KPK0 | Nicotiana attenuata | Yes | No |
| (23) | | | |
| A0A1U8INJ7 | Gossypium hirsutum | Yes | No |
| A2YDX4 | Oryza sativa subsp. indica | Yes | No |
| A2YRE8 | Oryza sativa subsp. indica (Rice) | No | Yes |
| A3A4W8 | Oryza sativa subsp. japonica | Yes | No |
| A3CBG3 | Oryza sativa subsp. japonica | Yes | No |
| D7MMG9 | Arabidopsis lyrata subsp. lyrata | Yes | No |
| (7) | | | |
| H6CS09 | Paenibacillus sp. Aloe-11 | Yes | No |
| LIKS92 | Streptomyces ipomoeae 91-03 | Yes | No |
| LIKUA4 | Streptomyces ipomoeae 91-03 | Yes | No |
| M4DIE5 | Brassica rapa subsp. pekinensis | Yes | Yes |
| (16) | | | |
| M5WQ23 | Prunus persica | Yes | Yes |
| M5X864 | Prunus persica | Yes | Yes |
| (31) | | | |
| O06997 | Bacillus subtilis (strain 168) | Yes | No |
| O64743 | Arabidopsis thaliana | No | Yes |
| O64745 | Arabidopsis thaliana | No | Yes |
| (37) | | | |
| P93479 | Papaver somniferum | Yes | Yes |
| (47) | | | |
| Q93ZA3 | Arabidopsis thaliana | No | Yes |
| Q9FKU8 | Arabidopsis thaliana | Yes | No |
| Q9FKU9 | Arabidopsis thaliana | Yes | No |
| Q9FKV2 | Arabidopsis thaliana | Yes | No |
| Q9FZC5 | Arabidopsis thaliana | Yes | No |
| Q9FZC7 | Arabidopsis thaliana | No | Yes |
| Q9SA86 | Arabidopsis thaliana | No | Yes |
| Q9SA89 | Arabidopsis thaliana | No | Yes |
| Q9SVG3 | Arabidopsis thaliana | No | Yes |
| Q9SVG4 | Arabidopsis thaliana | No | Yes |
| Q9SVG7 | Arabidopsis thaliana | No | Yes |
| V9EEP8 | Phytophthora parasitica P1569 | No | Yes |

Out of the 72 cannabinoid synthase orthologues tested, 20 showed the production of a cannabinoid with a molecular formula of C₂₂H₃₀O₄ (MW=358.5; m/z 357.5) and 16 orthologues showed the production of a cannabinoid with a molecular formula of C₂₂H₃₀O₄ (MW=374.5; m/z 373.5). Five orthologues showed the production of both types of cannabinoids. See Table 1 above.

### Example 3: Cannabinoid Synthase Orthologue Expression in Pichia pastoris

Genes encoding orthologues that showed cannabinoid synthase activity using CBGA as substrate were cloned into a P. pastoris expression system for larger scale protein expression. The expression of THCA synthase in P. pastoris was previously demonstrated. See, e.g., Zirpel et al., Biotechnology Lett. 2015, 37 (9): 1869-1875 and Lange et al., J. Biotechnol. 2015, 211:68-76.

The genes were cloned into the pPICZA plasmid (Invitrogen) and subsequently integrated into the P. pastoris genome using standard techniques. The resulting P. pastoris integrants were inoculated from agar plates and incubated in 20 mL buffered complex glycerol medium in baffled flasks at 30° C. for 48 h with shaking at 190 rpm. Cells were harvested by centrifugation and resuspended in 200 mL buffered methanol complex medium containing 0.5% w/v casamino acids and 0.01% w/v riboflavin. The expression of orthologue proteins was induced by adding 1% methanol every 24 h for a total of 120 hr.

Cells were harvested by centrifugation and resuspended in Buffer A (100 mM Tris, pH 8.0 and 150 mM NaCl). The cells were lysed using an M110P Microfluidizer® (Microfluidics International Corp.). Cell debris was removed by centrifugation and the cell lysates were loaded onto a 5 mL StrepTrap™ HP column (Cytiva Life Sciences) with a flow rate of 1 mL/min. The column was washed with 25 mL Buffer A, i.e., 5 column volumes (“CV”), and eluted with 6 CV Buffer B (100 mM Tris, pH 8.0, 150 mM NaCl, and 2.5 mM desthiobiotin). Fractions containing protein, identified by an increase in absorbance at 280 nm, were pooled, concentrated, and frozen at −80° C.

In a reaction volume of 50 μL, the cannabinoid synthase purified from was incubated with 150 μM CBGA and 0.2 mM FAD in 50 mM citrate, pH 8.0. The reaction was incubated for 24-48 h at 30° C., followed by the addition of 160 μL of acetonitrile to stop the reaction and precipitate the protein. The mixture (120 μL) was injected into an Agilent® InfinityLab® Poroshell® 120 EC-C₁₈ column for mass analysis using the Agilent® 1290 Infinity HPLC coupled with Agilent® 6550 iFunnel® Q-TOF high resolution mass spectrometer in the negative ion mode. EICs for m/z=357.2071, m/z=373.2020, and m/z=329.2122 were generated for each sample to determine if the biosynthesis of a potential cannabinoid was catalyzed by the orthologue.

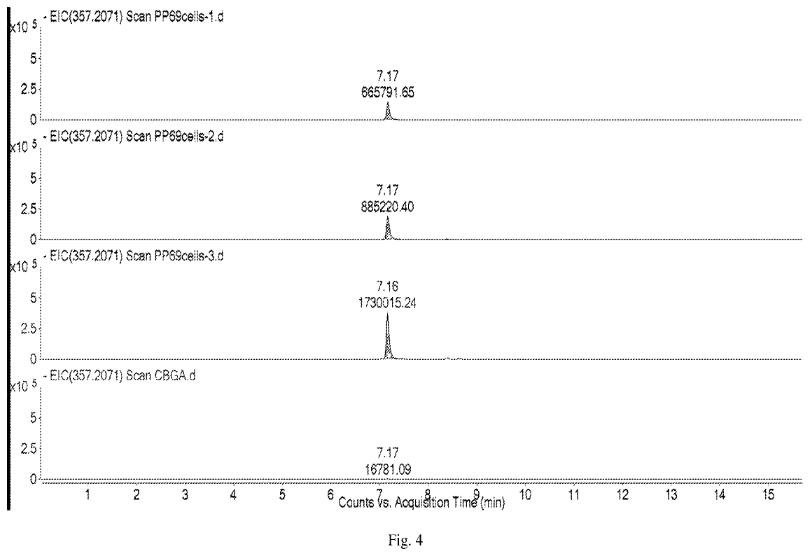

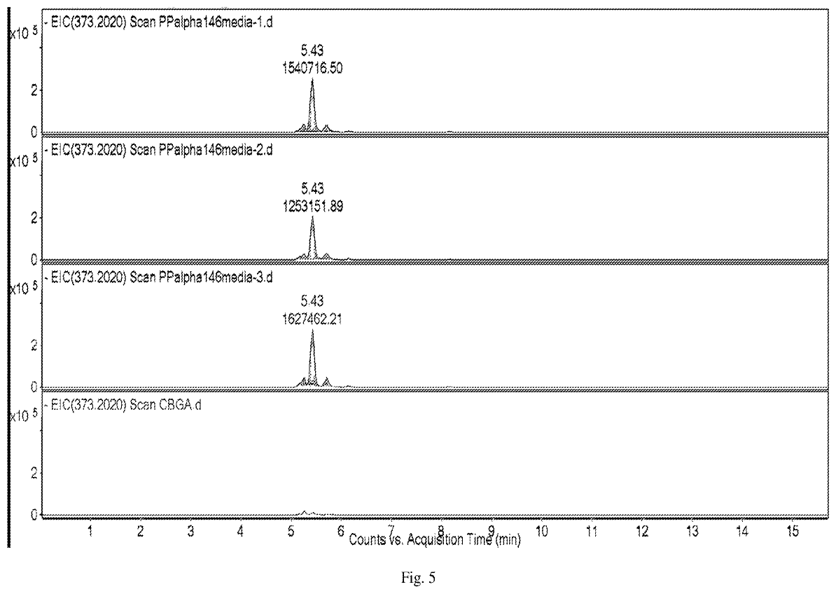

The results are shown in FIGS. 4-6 and Table 2 below.

TABLE 2
Activity of cannabinoid synthase orthologues expressed in P. pastoris

| UniProt ID | | m/z = | m/z = | m/z = |
|---|---|---|---|---|
| (SEQ ID NO) | Organism | 357.2071 | 373.2020 | 329.2122 |
| D7MMG9 | Arabidopsis lyrata subsp. | Yes | No | No |
| (7) | lyrata | | | |
| M4DIE5 | Brassica rapa subsp. | Yes | Yes | Yes |
| (16) | pekinensis | | | |
| A0A1J6KPK0 | Nicotiana attenuata | Yes | Yes | Yes |
| (23) | | | | |
| M5X864 | Prunus persica | Yes | No | No |
| (31) | | | | |
| O64745 | Arabidopsis thaliana | Yes | No | No |
| (37) | | | | |
| P93479 | Papaver somniferum | Yes | Yes | Yes |
| (47) | | | | |
| A0A1U8G0D2 | Capsicum annuum | Yes | Yes | Yes |
| (57) | | | | |
| A0A1S3XA06 | Nicotiana tabacum | No | Yes | Yes |
| (67) | | | | |
| A0A1S3BDA8 | Cucumis melo | No | Yes | Yes |
| (77) | | | | |
| A0A1U8FGM5 | Capsicum annuum | Yes | Yes | Yes |
| (87) | | | | |
| A0A1U8MHW2 | Gossypium hirsutum | No | Yes | Yes |
| (97) | (Gossypium mexicanum) | | | |
| A0A1J3CT99 | Noccaea caerulescens | Yes | Yes | Yes |
| (107) | (Thlaspi caerulescens) | | | |
| A0A1J3J6A2 | Noccaea caerulescens | Yes | Yes | Yes |
| (117) | (Thlaspi caerulescens) | | | |
| A0A1S3BE31 | Cucumis melo | No | Yes | Yes |
| (127) | | | | |

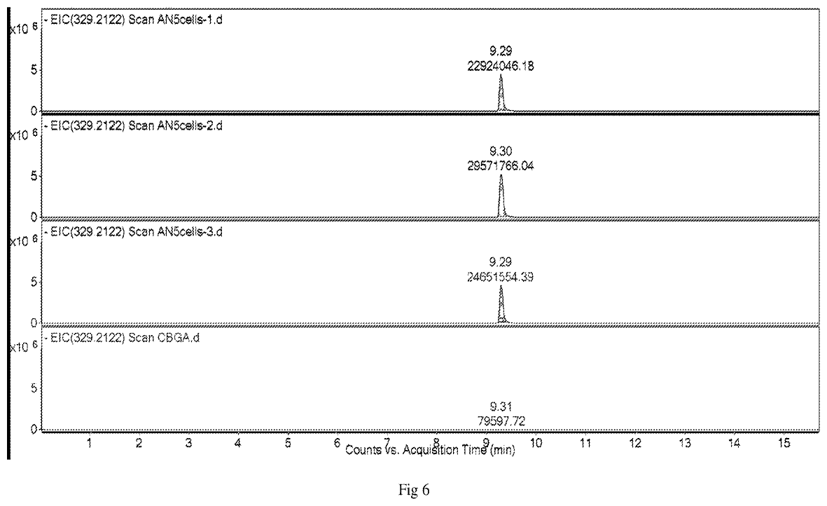

Out of the 232 potential CBS orthologs screened, seven orthologs showed the production of two types of cannabinoids, (i) the “cannabidiolic acid group” type having a molecular formula of C₂₂H₃₀O₄ (FW=358.5; m/z=357.2071, see FIG. 4) and (ii) cannabielsoic acid, with a molecular formula of C₂₂H₃₀O₄ (FW=374.5; m/z=373.2020, see FIG. 5). The decarboxylated product of cannabielsoic acid, namely, cannabielsoin, having a molecular formula of C₂₁H₃₀₀₂ (FW=330.5; m/z=329.2122, see FIG. 6) was also observed. Three orthologues produced only 255 cannabinoids of the cannabidiolic acid group. Four orthologues produced only cannabielsoic acid and cannabielsoin.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the 265 essential characteristics of the present invention, and without departing from the spirit and scope thereof, can make various changes and modifications of the invention to adapt it to various usages and conditions. Thus, other embodiments are also within the scope of the following claims.

What is claimed is:

1. A method for producing one or more cannabinoids, the method comprising contacting cannabigerolic acid (CBGA) with a cannabinoid synthase orthologue, wherein the orthologue includes an amino acid sequence selected from the group consisting of SEQ ID NOs: 7, 31, 37, 67, 77, 97, 117, and 127.

2. The method of claim 1, wherein the cannabinoids produced are selected from the group consisting of cannabidiolic acid, cannabielsoic acid, and cannabielsoin.

3. The method of claim 2, wherein the cannabinoids produced include cannabielsoic acid and cannabielsoin.

4. The method of claim 3, wherein the cannabinoids produced are free of cannabidiolic acid and the cannabinoid synthase orthologue includes the amino acid sequence of SEQ ID NOs: 67, 77, 97, or 127.

5. The method of claim 1, wherein the cannabinoid synthase orthologue is a recombinant enzyme produced in Saccharomyces cerevisiae, Yarrowia lipolytica, Kluyveromyces marxianus, or Pichia pastoris.

6. A recombinant cell of Saccharomyces cerevisiae or Pichia pastoris, comprising in its genome a nucleic acid encoding a cannabinoid synthase orthologue comprising an amino acid sequence selected from the group consisting of SEQ ID NOs: 7, 31, 37, 67, 77, 97, 117, and 127, and the cannabinoid synthase orthologue is expressed in the recombinant cell in an active form.

7. The recombinant cell of claim 6, wherein the nucleic acid encodes a cannabinoid synthase orthologue that includes the amino acid sequence of SEQ ID NOs: 67, 77, 97, or 127.


SEQUENCE LISTING (135 sequences)

SEQ ID NO: 1 - DNA, 24 bp, synthetic construct
  misc_feature 1..24: note = Strep Tag
  source 1..24: mol_type = other DNA
  CDS 1..24
tggtcacatccacaatttgaaaag

SEQ ID NO: 2 - protein, 8 aa, synthetic construct
  REGION 1..8: note = Synthetic Construct
WSHPQFEK

SEQ ID NO: 3 - DNA, 57 bp, synthetic construct
  misc_feature 1..57: note = truncated alpha factor
  source 1..57: mol_type = other DNA
  CDS 1..57
atgagatttccttccatcttcacggctgtgctatttgcagcatcctccgcacttgca

SEQ ID NO: 4 - protein, 19 aa, synthetic construct
  REGION 1..19: note = Synthetic Construct
MRFPSIFTAVLFAASSALA

SEQ ID NO: 5 - DNA, 267 bp, synthetic construct
  misc_feature 1..267: note = alpha factor full
  source 1..267: mol_type = other DNA
  CDS 1..267
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagct

SEQ ID NO: 6 - protein, 89 aa, synthetic construct
  REGION 1..89: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 7 - DNA, 1431 bp, synthetic construct
  misc_feature 1..1431: note = CBS1-D7MMG9
  source 1..1431: mol_type = other DNA
  CDS 1..1431
atgaaccacaacaccgttgaatccaaggttattcatacctccaaggactcttcattcttctccattttggactcctctattcagaacccaagattctctgtttctgaaactccaaagccagtttccattattactccagttaaggcctctgatgttcaaaccgttattagatgtgctagattgcatggtatccacgttagaactagatctgctggtcattgctatgaaggcttgtcttatattgcttacaacaagccattcgtcgtcatcgacattagaaacttgcaatccatctccttggatgttgataatagaactggttgggttcaaactggtgctactgctggtgaactatattacgaaattggtaagactccaaagaccttggcttttccagctggtattcatccaactgttgctgccgataacattattgatgctttggttgttgatgcctccggtagaattttggatagacaagctatgggtgaagagtactacttttgggctatttgcggtggtggtggttcttcttttggtattatcttgtcctggaagatcaagttggttgatgttccatctaccatcactgttttcaaggttaagaggacctctaagaaagaagccgttagaatcattaacaagtggcaatacgttgccgataaggttccagatgatttgttcattagaaccaccttggaaaggtctaacaagaatgctgttcatgctttgtttaccggcttgtatttgggtccagctaacaatttgttggccttgatggaagaaaagttcccagaattgggtttagaaaaggatggttgtactgaaatgtcctgggttgaatctgttttgtggtttgctgatttccacaagggtgaatctttggatgatgttttgaccaacagagaaaggacctctttgtcttacaaaggtaaggatgatttcgtgcaagagccaattccagaagctgctattcaagaactttggagaagattggatgctccagaagcaagattggccaagattattttgactccatttggtggtaagatgtccgaaattgctgaacacgaaactttgttcccacatagagaaggtaacttgtacgaaattcaatacgtcgcctattggagagaagaagaggataagaatatgactggcaccaacaaatacttgaagtgggttgattcagtctacgaattgatgactccatacgtttctaaatctccaagaggtgcttacgttaacttcgttgatatggatttgggtatgtacctgggtaagaaaaagaccaaatacgaagagggtaaatcctggggtgttaagtactttaagaacaacttcgaaagactggtcagagtcaagacttctgttgatccaactgatttcttctgcgacgaacaatctattcccttgttgaacaaagtcact

SEQ ID NO: 8 - protein, 477 aa, synthetic construct
  REGION 1..477: note = Synthetic Construct
MNHNTVESKVIHTSKDSSFFSILDSSIQNPRFSVSETPKPVSIITPVKASDVQTVIRCARLHGIHVRTRSAGHCYEGLSYIAYNKPFVVIDIRNLQSISLDVDNRTGWVQTGATAGELYYEIGKTPKTLAFPAGIHPTVAADNIIDALVVDASGRILDRQAMGEEYYFWAICGGGGSSFGIILSWKIKLVDVPSTITVFKVKRTSKKEAVRIINKWQYVADKVPDDLFIRTTLERSNKNAVHALFTGLYLGPANNLLALMEEKFPELGLEKDGCTEMSWVESVLWFADFHKGESLDDVLTNRERTSLSYKGKDDFVQEPIPEAAIQELWRRLDAPEARLAKIILTPFGGKMSEIAEHETLFPHREGNLYEIQYVAYWREEEDKNMTGTNKYLKWVDSVYELMTPYVSKSPRGAYVNFVDMDLGMYLGKKKTKYEEGKSWGVKYFKNNFERLVRVKTSVDPTDFFCDEQSIPLLNKVT

SEQ ID NO: 9 - DNA, 1455 bp, synthetic construct
  misc_feature 1..1455: note = CBS1-D7MMG9-strep-Tag
  source 1..1455: mol_type = other DNA
  CDS 1..1455
atgaaccacaacaccgttgaatccaaggttattcatacctccaaggactcttcattcttctccattttggactcctctattcagaacccaagattctctgtttctgaaactccaaagccagtttccattattactccagttaaggcctctgatgttcaaaccgttattagatgtgctagattgcatggtatccacgttagaactagatctgctggtcattgctatgaaggcttgtcttatattgcttacaacaagccattcgtcgtcatcgacattagaaacttgcaatccatctccttggatgttgataatagaactggttgggttcaaactggtgctactgctggtgaactatattacgaaattggtaagactccaaagaccttggcttttccagctggtattcatccaactgttgctgccgataacattattgatgctttggttgttgatgcctccggtagaattttggatagacaagctatgggtgaagagtactacttttgggctatttgcggtggtggtggttcttcttttggtattatcttgtcctggaagatcaagttggttgatgttccatctaccatcactgttttcaaggttaagaggacctctaagaaagaagccgttagaatcattaacaagtggcaatacgttgccgataaggttccagatgatttgttcattagaaccaccttggaaaggtctaacaagaatgctgttcatgctttgtttaccggcttgtatttgggtccagctaacaatttgttggccttgatggaagaaaagttcccagaattgggtttagaaaaggatggttgtactgaaatgtcctgggttgaatctgttttgtggtttgctgatttccacaagggtgaatctttggatgatgttttgaccaacagagaaaggacctctttgtcttacaaaggtaaggatgatttcgtgcaagagccaattccagaagctgctattcaagaactttggagaagattggatgctccagaagcaagattggccaagattattttgactccatttggtggtaagatgtccgaaattgctgaacacgaaactttgttcccacatagagaaggtaacttgtacgaaattcaatacgtcgcctattggagagaagaagaggataagaatatgactggcaccaacaaatacttgaagtgggttgattcagtctacgaattgatgactccatacgtttctaaatctccaagaggtgcttacgttaacttcgttgatatggatttgggtatgtacctgggtaagaaaaagaccaaatacgaagagggtaaatcctggggtgttaagtactttaagaacaacttcgaaagactggtcagagtcaagacttctgttgatccaactgatttcttctgcgacgaacaatctattcccttgttgaacaaagtcacttggtcacatccacaatttgaaaag

SEQ ID NO: 10 - protein, 485 aa, synthetic construct
  REGION 1..485: note = Synthetic Construct
MNHNTVESKVIHTSKDSSFFSILDSSIQNPRFSVSETPKPVSIITPVKASDVQTVIRCARLHGIHVRTRSAGHCYEGLSYIAYNKPFVVIDIRNLQSISLDVDNRTGWVQTGATAGELYYEIGKTPKTLAFPAGIHPTVAADNIIDALVVDASGRILDRQAMGEEYYFWAICGGGGSSFGIILSWKIKLVDVPSTITVFKVKRTSKKEAVRIINKWQYVADKVPDDLFIRTTLERSNKNAVHALFTGLYLGPANNLLALMEEKFPELGLEKDGCTEMSWVESVLWFADFHKGESLDDVLTNRERTSLSYKGKDDFVQEPIPEAAIQELWRRLDAPEARLAKIILTPFGGKMSEIAEHETLFPHREGNLYEIQYVAYWREEEDKNMTGTNKYLKWVDSVYELMTPYVSKSPRGAYVNFVDMDLGMYLGKKKTKYEEGKSWGVKYFKNNFERLVRVKTSVDPTDFFCDEQSIPLLNKVTWSHPQFEK

SEQ ID NO: 11 - DNA, 1512 bp, synthetic construct
  misc_feature 1..1512: note = CBS1-D7MMG9- truncated alpha factor strep Tag
  source 1..1512: mol_type = other DNA
  CDS 1..1512
atgagatttccttccatcttcacggctgtgctatttgcagcatcctccgcacttgcaatgaaccacaacaccgttgaatccaaggttattcatacctccaaggactcttcattcttctccattttggactcctctattcagaacccaagattctctgtttctgaaactccaaagccagtttccattattactccagttaaggcctctgatgttcaaaccgttattagatgtgctagattgcatggtatccacgttagaactagatctgctggtcattgctatgaaggcttgtcttatattgcttacaacaagccattcgtcgtcatcgacattagaaacttgcaatccatctccttggatgttgataatagaactggttgggttcaaactggtgctactgctggtgaactatattacgaaattggtaagactccaaagaccttggcttttccagctggtattcatccaactgttgctgccgataacattattgatgctttggttgttgatgcctccggtagaattttggatagacaagctatgggtgaagagtactacttttgggctatttgcggtggtggtggttcttcttttggtattatcttgtcctggaagatcaagttggttgatgttccatctaccatcactgttttcaaggttaagaggacctctaagaaagaagccgttagaatcattaacaagtggcaatacgttgccgataaggttccagatgatttgttcattagaaccaccttggaaaggtctaacaagaatgctgttcatgctttgtttaccggcttgtatttgggtccagctaacaatttgttggccttgatggaagaaaagttcccagaattgggtttagaaaaggatggttgtactgaaatgtcctgggttgaatctgttttgtggtttgctgatttccacaagggtgaatctttggatgatgttttgaccaacagagaaaggacctctttgtcttacaaaggtaaggatgatttcgtgcaagagccaattccagaagctgctattcaagaactttggagaagattggatgctccagaagcaagattggccaagattattttgactccatttggtggtaagatgtccgaaattgctgaacacgaaactttgttcccacatagagaaggtaacttgtacgaaattcaatacgtcgcctattggagagaagaagaggataagaatatgactggcaccaacaaatacttgaagtgggttgattcagtctacgaattgatgactccatacgtttctaaatctccaagaggtgcttacgttaacttcgttgatatggatttgggtatgtacctgggtaagaaaaagaccaaatacgaagagggtaaatcctggggtgttaagtactttaagaacaacttcgaaagactggtcagagtcaagacttctgttgatccaactgatttcttctgcgacgaacaatctattcccttgttgaacaaagtcacttggtcacatccacaatttgaaaag

SEQ ID NO: 12 - protein, 504 aa, synthetic construct
  REGION 1..504: note = Synthetic Construct
MRFPSIFTAVLFAASSALAMNHNTVESKVIHTSKDSSFFSILDSSIQNPRFSVSETPKPVSIITPVKASDVQTVIRCARLHGIHVRTRSAGHCYEGLSYIAYNKPFVVIDIRNLQSISLDVDNRTGWVQTGATAGELYYEIGKTPKTLAFPAGIHPTVAADNIIDALVVDASGRILDRQAMGEEYYFWAICGGGGSSFGIILSWKIKLVDVPSTITVFKVKRTSKKEAVRIINKWQYVADKVPDDLFIRTTLERSNKNAVHALFTGLYLGPANNLLALMEEKFPELGLEKDGCTEMSWVESVLWFADFHKGESLDDVLTNRERTSLSYKGKDDFVQEPIPEAAIQELWRRLDAPEARLAKIILTPFGGKMSEIAEHETLFPHREGNLYEIQYVAYWREEEDKNMTGTNKYLKWVDSVYELMTPYVSKSPRGAYVNFVDMDLGMYLGKKKTKYEEGKSWGVKYFKNNFERLVRVKTSVDPTDFFCDEQSIPLLNKVTWSHPQFEK

SEQ ID NO: 13 - DNA, 1722 bp, synthetic construct
  misc_feature 1..1722: note = CBS1-D7MMG9-full alpha factor -strep Tag
  source 1..1722: mol_type = other DNA
  CDS 1..1722
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatgaaccacaacaccgttgaatccaaggttattcatacctccaaggactcttcattcttctccattttggactcctctattcagaacccaagattctctgtttctgaaactccaaagccagtttccattattactccagttaaggcctctgatgttcaaaccgttattagatgtgctagattgcatggtatccacgttagaactagatctgctggtcattgctatgaaggcttgtcttatattgcttacaacaagccattcgtcgtcatcgacattagaaacttgcaatccatctccttggatgttgataatagaactggttgggttcaaactggtgctactgctggtgaactatattacgaaattggtaagactccaaagaccttggcttttccagctggtattcatccaactgttgctgccgataacattattgatgctttggttgttgatgcctccggtagaattttggatagacaagctatgggtgaagagtactacttttgggctatttgcggtggtggtggttcttcttttggtattatcttgtcctggaagatcaagttggttgatgttccatctaccatcactgttttcaaggttaagaggacctctaagaaagaagccgttagaatcattaacaagtggcaatacgttgccgataaggttccagatgatttgttcattagaaccaccttggaaaggtctaacaagaatgctgttcatgctttgtttaccggcttgtatttgggtccagctaacaatttgttggccttgatggaagaaaagttcccagaattgggtttagaaaaggatggttgtactgaaatgtcctgggttgaatctgttttgtggtttgctgatttccacaagggtgaatctttggatgatgttttgaccaacagagaaaggacctctttgtcttacaaaggtaaggatgatttcgtgcaagagccaattccagaagctgctattcaagaactttggagaagattggatgctccagaagcaagattggccaagattattttgactccatttggtggtaagatgtccgaaattgctgaacacgaaactttgttcccacatagagaaggtaacttgtacgaaattcaatacgtcgcctattggagagaagaagaggataagaatatgactggcaccaacaaatacttgaagtgggttgattcagtctacgaattgatgactccatacgtttctaaatctccaagaggtgcttacgttaacttcgttgatatggatttgggtatgtacctgggtaagaaaaagaccaaatacgaagagggtaaatcctggggtgttaagtactttaagaacaacttcgaaagactggtcagagtcaagacttctgttgatccaactgatttcttctgcgacgaacaatctattcccttgttgaacaaagtcacttggtcacatccacaatttgaaaag

SEQ ID NO: 14 - protein, 574 aa, synthetic construct
  REGION 1..574: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAMNHNTVESKVIHTSKDSSFFSILDSSIQNPRFSVSETPKPVSIITPVKASDVQTVIRCARLHGIHVRTRSAGHCYEGLSYIAYNKPFVVIDIRNLQSISLDVDNRTGWVQTGATAGELYYEIGKTPKTLAFPAGIHPTVAADNIIDALVVDASGRILDRQAMGEEYYFWAICGGGGSSFGIILSWKIKLVDVPSTITVFKVKRTSKKEAVRIINKWQYVADKVPDDLFIRTTLERSNKNAVHALFTGLYLGPANNLLALMEEKFPELGLEKDGCTEMSWVESVLWFADFHKGESLDDVLTNRERTSLSYKGKDDFVQEPIPEAAIQELWRRLDAPEARLAKIILTPFGGKMSEIAEHETLFPHREGNLYEIQYVAYWREEEDKNMTGTNKYLKWVDSVYELMTPYVSKSPRGAYVNFVDMDLGMYLGKKKTKYEEGKSWGVKYFKNNFERLVRVKTSVDPTDFFCDEQSIPLLNKVTWSHPQFEK

SEQ ID NO: 15 - DNA, 1011 bp, synthetic construct
  misc_feature 1..1011: note = CBS5-M4DIE5
  source 1..1011: mol_type = other DNA
  CDS 1..1011
atgaggaagtacggtttgaccgttgataatgttgttgatgccttgatcgttgatgttaacggcaagttgttgaacagatctactatgggtgaagatttgttctgggctattagaggtggtggtggcggttcttttggtgttattttgtcttggaagatcaacttggttgacgtcccagaaattttgactgttttcagagttaacaagaccttggaacaaggtggtattgacgtcttgtacaaatggcaattggtgtcctctaagttgttggatggtttgtttttgagagccttgtctcaagttgtcaacagaactatcaccgttgaattttacgctcagtttttgggtagagccgatgaattggttgctatcgttaatcaatctttgccagagttgggtttgaagagacaagattgcttggaaatgtcttggttgaacactaccttgttttgggaagatttgccagttggtactccaacttctgttttgttggatagaccatctaagccagagaagttcttcaagtctaaatccgattacgtcaagaagcccattccaaaagagggtattaagaaattgtgggaagccttgttggagtttaacaacaacaatatcgtctacatgcagtggaatccatacggtggtgttatggatactgttccagctgctgctactccatttccacatagaaaaggtaacttgttcgaagtccagtactacacttcttggttggatgctaatgctacaatgggttctttggacatgatgaagaagttgtacgaagttgctgaaccctacgtttcttctaatccaagagaagcctttttgaactacagagacatcgacattggtagcaatccatctaacgaaactaatgttgaagaagccgaaatctacggctctaagtactttgttggtaacttgaagagattgatggaagtcaaggctaagtacgatccagaaaactttttcaagaacgagcaatctattccaccagccagagttaagcaa

SEQ ID NO: 16 - protein, 337 aa, synthetic construct
  REGION 1..337: note = Synthetic Construct
MRKYGLTVDNVVDALIVDVNGKLLNRSTMGEDLFWAIRGGGGGSFGVILSWKINLVDVPEILTVFRVNKTLEQGGIDVLYKWQLVSSKLLDGLFLRALSQVVNRTITVEFYAQFLGRADELVAIVNQSLPELGLKRQDCLEMSWLNTTLFWEDLPVGTPTSVLLDRPSKPEKFFKSKSDYVKKPIPKEGIKKLWEALLEFNNNNIVYMQWNPYGGVMDTVPAAATPFPHRKGNLFEVQYYTSWLDANATMGSLDMMKKLYEVAEPYVSSNPREAFLNYRDIDIGSNPSNETNVEEAEIYGSKYFVGNLKRLMEVKAKYDPENFFKNEQSIPPARVKQ

SEQ ID NO: 17 - DNA, 1035 bp, synthetic construct
  misc_feature 1..1035: note = CBS5-M4DIE5 strep Tag
  source 1..1035: mol_type = other DNA
  CDS 1..1035
atgaggaagtacggtttgaccgttgataatgttgttgatgccttgatcgttgatgttaacggcaagttgttgaacagatctactatgggtgaagatttgttctgggctattagaggtggtggtggcggttcttttggtgttattttgtcttggaagatcaacttggttgacgtcccagaaattttgactgttttcagagttaacaagaccttggaacaaggtggtattgacgtcttgtacaaatggcaattggtgtcctctaagttgttggatggtttgtttttgagagccttgtctcaagttgtcaacagaactatcaccgttgaattttacgctcagtttttgggtagagccgatgaattggttgctatcgttaatcaatctttgccagagttgggtttgaagagacaagattgcttggaaatgtcttggttgaacactaccttgttttgggaagatttgccagttggtactccaacttctgttttgttggatagaccatctaagccagagaagttcttcaagtctaaatccgattacgtcaagaagcccattccaaaagagggtattaagaaattgtgggaagccttgttggagtttaacaacaacaatatcgtctacatgcagtggaatccatacggtggtgttatggatactgttccagctgctgctactccatttccacatagaaaaggtaacttgttcgaagtccagtactacacttcttggttggatgctaatgctacaatgggttctttggacatgatgaagaagttgtacgaagttgctgaaccctacgtttcttctaatccaagagaagcctttttgaactacagagacatcgacattggtagcaatccatctaacgaaactaatgttgaagaagccgaaatctacggctctaagtactttgttggtaacttgaagagattgatggaagtcaaggctaagtacgatccagaaaactttttcaagaacgagcaatctattccaccagccagagttaagcaatggtcacatccacaatttgaaaag

SEQ ID NO: 18 - protein, 345 aa, synthetic construct
  REGION 1..345: note = Synthetic Construct
MRKYGLTVDNVVDALIVDVNGKLLNRSTMGEDLFWAIRGGGGGSFGVILSWKINLVDVPEILTVFRVNKTLEQGGIDVLYKWQLVSSKLLDGLFLRALSQVVNRTITVEFYAQFLGRADELVAIVNQSLPELGLKRQDCLEMSWLNTTLFWEDLPVGTPTSVLLDRPSKPEKFFKSKSDYVKKPIPKEGIKKLWEALLEFNNNNIVYMQWNPYGGVMDTVPAAATPFPHRKGNLFEVQYYTSWLDANATMGSLDMMKKLYEVAEPYVSSNPREAFLNYRDIDIGSNPSNETNVEEAEIYGSKYFVGNLKRLMEVKAKYDPENFFKNEQSIPPARVKQWSHPQFEK

SEQ ID NO: 19 - DNA, 1092 bp, synthetic construct
  misc_feature 1..1092: note = CBS5-M4DIE5-truncated alpha factor strep Tag
  source 1..1092: mol_type = other DNA
atgagatttccttccatcttcacggctgtgctatttgcagcatcctccgcacttgcaatgaggaagtacggtttgaccgttgataatgttgttgatgccttgatcgttgatgttaacggcaagttgttgaacagatctactatgggtgaagatttgttctgggctattagaggtggtggtggcggttcttttggtgttattttgtcttggaagatcaacttggttgacgtcccagaaattttgactgttttcagagttaacaagaccttggaacaaggtggtattgacgtcttgtacaaatggcaattggtgtcctctaagttgttggatggtttgtttttgagagccttgtctcaagttgtcaacagaactatcaccgttgaattttacgctcagtttttgggtagagccgatgaattggttgctatcgttaatcaatctttgccagagttgggtttgaagagacaagattgcttggaaatgtcttggttgaacactaccttgttttgggaagatttgccagttggtactccaacttctgttttgttggatagaccatctaagccagagaagttcttcaagtctaaatccgattacgtcaagaagcccattccaaaagagggtattaagaaattgtgggaagccttgttggagtttaacaacaacaatatcgtctacatgcagtggaatccatacggtggtgttatggatactgttccagctgctgctactccatttccacatagaaaaggtaacttgttcgaagtccagtactacacttcttggttggatgctaatgctacaatgggttctttggacatgatgaagaagttgtacgaagttgctgaaccctacgtttcttctaatccaagagaagcctttttgaactacagagacatcgacattggtagcaatccatctaacgaaactaatgttgaagaagccgaaatctacggctctaagtactttgttggtaacttgaagagattgatggaagtcaaggctaagtacgatccagaaaactttttcaagaacgagcaatctattccaccagccagagttaagcaatggtcacatccacaatttgaaaag

SEQ ID NO: 20 - DNA, 1302 bp, synthetic construct
  misc_feature 1..1302: note = CBS5-M4DIE5-nucleotide-full alpha factor strep Tag
  source 1..1302: mol_type = other DNA
  CDS 1..1302
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatgaggaagtacggtttgaccgttgataatgttgttgatgccttgatcgttgatgttaacggcaagttgttgaacagatctactatgggtgaagatttgttctgggctattagaggtggtggtggcggttcttttggtgttattttgtcttggaagatcaacttggttgacgtcccagaaattttgactgttttcagagttaacaagaccttggaacaaggtggtattgacgtcttgtacaaatggcaattggtgtcctctaagttgttggatggtttgtttttgagagccttgtctcaagttgtcaacagaactatcaccgttgaattttacgctcagtttttgggtagagccgatgaattggttgctatcgttaatcaatctttgccagagttgggtttgaagagacaagattgcttggaaatgtcttggttgaacactaccttgttttgggaagatttgccagttggtactccaacttctgttttgttggatagaccatctaagccagagaagttcttcaagtctaaatccgattacgtcaagaagcccattccaaaagagggtattaagaaattgtgggaagccttgttggagtttaacaacaacaatatcgtctacatgcagtggaatccatacggtggtgttatggatactgttccagctgctgctactccatttccacatagaaaaggtaacttgttcgaagtccagtactacacttcttggttggatgctaatgctacaatgggttctttggacatgatgaagaagttgtacgaagttgctgaaccctacgtttcttctaatccaagagaagcctttttgaactacagagacatcgacattggtagcaatccatctaacgaaactaatgttgaagaagccgaaatctacggctctaagtactttgttggtaacttgaagagattgatggaagtcaaggctaagtacgatccagaaaactttttcaagaacgagcaatctattccaccagccagagttaagcaatggtcacatccacaatttgaaaag

SEQ ID NO: 21 - protein, 434 aa, synthetic construct
  REGION 1..434: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAMRKYGLTVDNVVDALIVDVNGKLLNRSTMGEDLFWAIRGGGGGSFGVILSWKINLVDVPEILTVFRVNKTLEQGGIDVLYKWQLVSSKLLDGLFLRALSQVVNRTITVEFYAQFLGRADELVAIVNQSLPELGLKRQDCLEMSWLNTTLFWEDLPVGTPTSVLLDRPSKPEKFFKSKSDYVKKPIPKEGIKKLWEALLEFNNNNIVYMQWNPYGGVMDTVPAAATPFPHRKGNLFEVQYYTSWLDANATMGSLDMMKKLYEVAEPYVSSNPREAFLNYRDIDIGSNPSNETNVEEAEIYGSKYFVGNLKRLMEVKAKYDPENFFKNEQSIPPARVKQWSHPQFEK

SEQ ID NO: 22 - DNA, 1641 bp, synthetic construct
  misc_feature 1..1641: note = CBS17-A0A1J6KPK0
  source 1..1641: mol_type = other DNA
  CDS 1..1641
atggcctccttgaactctatcatggtgtcttttttcttggtcctgctgttctcttcttcttgttttgctttgtctgataccactccagaaaagttctaccaatgtatctgcaagaactccgatttctctgatccattttctaaggctttcttcactccaaacaccgattctttcaacaccgttttaaactctaccgctcaaaacttgagatgtttgatgccaactgttccaaagccagttttgatttttactccaatggccgaatctcatgttcaagctgctgttatttgtgccaagcaattgaacttgcaattgagagttagatccggtggtcatgattatgaaggtttgtcctacatctccgttatggattctccattcgttatcgttgacttgtccaagttgagagagattgacgttaacatcgaagaaaattctgcttgggctcaagctggtgctactgttggtgaagtttattacagaatctccgagaagtctaagactcatggttttccagctggtttgtgtacctctttaggtattggtggtcatatcactggtggtgcttatggttctatgatgagaaaatttggtttgggtgccgataacgtcattgatgctagaatcgttaatgccaacggtactatcttggacagattgtctatgggtgaagatttgttctgggctattagaggtggtggtggcgcttcttttggtattatcttgtcttggaagttgaagttggttaccgttccatctgttgttactgttttcaccgttccaaaaaccttggaagatggtgctacaaagatcttgtacaaatggcaacaagttgccgataagatcgatgatgatttgttcatgagagtcgttatcaacgtcgttgacaaaaaggataagaagggtgaaagaactgttcaaaccgcttacaactctttgttcttgggtaatgttgacaggttgttgcagatcatgaatgaatcttttccagagttgggtttgacccaaaaagattgcactgaaatgtcctggatcgagtccatcttgtatattgctggtttcccaacaaaaaccccaccagaaattttgttgcaaggtaagccaactttcaagaactacttcaaggctaagtccgacttcatcaaagaacctattccagaaactggtctggatggtatttggaaaagactattggaagaggactccccattgatgatatggaatccatacggtggtatgatggccaacatttctgaatctgaaactccatttccacacagaaagggtgttatcttcaagatccaatacttgactttgtggaccgaaccagatcaagaattggctacaaaacatgttgactggatcagaaagctgtactcttacatgacttcttacgcttctatgtttccaagagaagcctacgttaactacagagatttggacttgggtatgaacaagaatggcaactctaactcctcattcgttcaagctacagtttggggtaacaagtacttcaacaacaatttcaacaggctggtcgaaatcaagactaaggttgatccagagaatttcttcaagttcgaacaatccattccaaccttgccattgactaagaaaagaaagggtaagaagatcaaccat

SEQ ID NO: 23 - protein, 547 aa, synthetic construct
  REGION 1..547: note = Synthetic Construct
MASLNSIMVSFFLVLLFSSSCFALSDTTPEKFYQCICKNSDFSDPFSKAFFTPNTDSFNTVLNSTAQNLRCLMPTVPKPVLIFTPMAESHVQAAVICAKQLNLQLRVRSGGHDYEGLSYISVMDSPFVIVDLSKLREIDVNIEENSAWAQAGATVGEVYYRISEKSKTHGFPAGLCTSLGIGGHITGGAYGSMMRKFGLGADNVIDARIVNANGTILDRLSMGEDLFWAIRGGGGASFGIILSWKLKLVTVPSVVTVFTVPKTLEDGATKILYKWQQVADKIDDDLFMRVVINVVDKKDKKGERTVQTAYNSLFLGNVDRLLQIMNESFPELGLTQKDCTEMSWIESILYIAGFPTKTPPEILLQGKPTFKNYFKAKSDFIKEPIPETGLDGIWKRLLEEDSPLMIWNPYGGMMANISESETPFPHRKGVIFKIQYLTLWTEPDQELATKHVDWIRKLYSYMTSYASMFPREAYVNYRDLDLGMNKNGNSNSSFVQATVWGNKYFNNNFNRLVEIKTKVDPENFFKFEQSIPTLPLTKKRKGKKINH

SEQ ID NO: 24 - DNA, 1575 bp, synthetic construct
  misc_feature 1..1575: note = CBS17-A0A1J6KPK0-no signal peptide
  source 1..1575: mol_type = other DNA
  CDS 1..1575
atgttgtctgataccactccagaaaagttctaccaatgtatctgcaagaactccgatttctctgatccattttctaaggctttcttcactccaaacaccgattctttcaacaccgttttaaactctaccgctcaaaacttgagatgtttgatgccaactgttccaaagccagttttgatttttactccaatggccgaatctcatgttcaagctgctgttatttgtgccaagcaattgaacttgcaattgagagttagatccggtggtcatgattatgaaggtttgtcctacatctccgttatggattctccattcgttatcgttgacttgtccaagttgagagagattgacgttaacatcgaagaaaattctgcttgggctcaagctggtgctactgttggtgaagtttattacagaatctccgagaagtctaagactcatggttttccagctggtttgtgtacctctttaggtattggtggtcatatcactggtggtgcttatggttctatgatgagaaaatttggtttgggtgccgataacgtcattgatgctagaatcgttaatgccaacggtactatcttggacagattgtctatgggtgaagatttgttctgggctattagaggtggtggtggcgcttcttttggtattatcttgtcttggaagttgaagttggttaccgttccatctgttgttactgttttcaccgttccaaaaaccttggaagatggtgctacaaagatcttgtacaaatggcaacaagttgccgataagatcgatgatgatttgttcatgagagtcgttatcaacgtcgttgacaaaaaggataagaagggtgaaagaactgttcaaaccgcttacaactctttgttcttgggtaatgttgacaggttgttgcagatcatgaatgaatcttttccagagttgggtttgacccaaaaagattgcactgaaatgtcctggatcgagtccatcttgtatattgctggtttcccaacaaaaaccccaccagaaattttgttgcaaggtaagccaactttcaagaactacttcaaggctaagtccgacttcatcaaagaacctattccagaaactggtctggatggtatttggaaaagactattggaagaggactccccattgatgatatggaatccatacggtggtatgatggccaacatttctgaatctgaaactccatttccacacagaaagggtgttatcttcaagatccaatacttgactttgtggaccgaaccagatcaagaattggctacaaaacatgttgactggatcagaaagctgtactcttacatgacttcttacgcttctatgtttccaagagaagcctacgttaactacagagatttggacttgggtatgaacaagaatggcaactctaactcctcattcgttcaagctacagtttggggtaacaagtacttcaacaacaatttcaacaggctggtcgaaatcaagactaaggttgatccagagaatttcttcaagttcgaacaatccattccaaccttgccattgactaagaaaagaaagggtaagaagatcaaccat

SEQ ID NO: 25 - protein, 525 aa, synthetic construct
  REGION 1..525: note = Synthetic Construct
MLSDTTPEKFYQCICKNSDFSDPFSKAFFTPNTDSFNTVLNSTAQNLRCLMPTVPKPVLIFTPMAESHVQAAVICAKQLNLQLRVRSGGHDYEGLSYISVMDSPFVIVDLSKLREIDVNIEENSAWAQAGATVGEVYYRISEKSKTHGFPAGLCTSLGIGGHITGGAYGSMMRKFGLGADNVIDARIVNANGTILDRLSMGEDLFWAIRGGGGASFGIILSWKLKLVTVPSVVTVFTVPKTLEDGATKILYKWQQVADKIDDDLFMRVVINVVDKKDKKGERTVQTAYNSLFLGNVDRLLQIMNESFPELGLTQKDCTEMSWIESILYIAGFPTKTPPEILLQGKPTFKNYFKAKSDFIKEPIPETGLDGIWKRLLEEDSPLMIWNPYGGMMANISESETPFPHRKGVIFKIQYLTLWTEPDQELATKHVDWIRKLYSYMTSYASMFPREAYVNYRDLDLGMNKNGNSNSSFVQATVWGNKYFNNNFNRLVEIKTKVDPENFFKFEQSIPTLPLTKKRKGKKINH

SEQ ID NO: 26 - DNA, 1599 bp, synthetic construct
  misc_feature 1..1599: note = CBS17-A0A1J6KPK0-no signal peptide-strep Tag
  source 1..1599: mol_type = other DNA
atgttgtctgataccactccagaaaagttctaccaatgtatctgcaagaactccgatttctctgatccattttctaaggctttcttcactccaaacaccgattctttcaacaccgttttaaactctaccgctcaaaacttgagatgtttgatgccaactgttccaaagccagttttgatttttactccaatggccgaatctcatgttcaagctgctgttatttgtgccaagcaattgaacttgcaattgagagttagatccggtggtcatgattatgaaggtttgtcctacatctccgttatggattctccattcgttatcgttgacttgtccaagttgagagagattgacgttaacatcgaagaaaattctgcttgggctcaagctggtgctactgttggtgaagtttattacagaatctccgagaagtctaagactcatggttttccagctggtttgtgtacctctttaggtattggtggtcatatcactggtggtgcttatggttctatgatgagaaaatttggtttgggtgccgataacgtcattgatgctagaatcgttaatgccaacggtactatcttggacagattgtctatgggtgaagatttgttctgggctattagaggtggtggtggcgcttcttttggtattatcttgtcttggaagttgaagttggttaccgttccatctgttgttactgttttcaccgttccaaaaaccttggaagatggtgctacaaagatcttgtacaaatggcaacaagttgccgataagatcgatgatgatttgttcatgagagtcgttatcaacgtcgttgacaaaaaggataagaagggtgaaagaactgttcaaaccgcttacaactctttgttcttgggtaatgttgacaggttgttgcagatcatgaatgaatcttttccagagttgggtttgacccaaaaagattgcactgaaatgtcctggatcgagtccatcttgtatattgctggtttcccaacaaaaaccccaccagaaattttgttgcaaggtaagccaactttcaagaactacttcaaggctaagtccgacttcatcaaagaacctattccagaaactggtctggatggtatttggaaaagactattggaagaggactccccattgatgatatggaatccatacggtggtatgatggccaacatttctgaatctgaaactccatttccacacagaaagggtgttatcttcaagatccaatacttgactttgtggaccgaaccagatcaagaattggctacaaaacatgttgactggatcagaaagctgtactcttacatgacttcttacgcttctatgtttccaagagaagcctacgttaactacagagatttggacttgggtatgaacaagaatggcaactctaactcctcattcgttcaagctacagtttggggtaacaagtacttcaacaacaatttcaacaggctggtcgaaatcaagactaaggttgatccagagaatttcttcaagttcgaacaatccattccaaccttgccattgactaagaaaagaaagggtaagaagatcaaccattggtcacatccacaatttgaaaag

SEQ ID NO: 27 - DNA, 1656 bp, synthetic construct
  misc_feature 1..1656: note = CBS17-A0A1J6KPK0-truncatd alpha factor - no signal peptide-strep Tag
  source 1..1656: mol_type = other DNA
atgagatttccttccatcttcacggctgtgctatttgcagcatcctccgcacttgcaatgttgtctgataccactccagaaaagttctaccaatgtatctgcaagaactccgatttctctgatccattttctaaggctttcttcactccaaacaccgattctttcaacaccgttttaaactctaccgctcaaaacttgagatgtttgatgccaactgttccaaagccagttttgatttttactccaatggccgaatctcatgttcaagctgctgttatttgtgccaagcaattgaacttgcaattgagagttagatccggtggtcatgattatgaaggtttgtcctacatctccgttatggattctccattcgttatcgttgacttgtccaagttgagagagattgacgttaacatcgaagaaaattctgcttgggctcaagctggtgctactgttggtgaagtttattacagaatctccgagaagtctaagactcatggttttccagctggtttgtgtacctctttaggtattggtggtcatatcactggtggtgcttatggttctatgatgagaaaatttggtttgggtgccgataacgtcattgatgctagaatcgttaatgccaacggtactatcttggacagattgtctatgggtgaagatttgttctgggctattagaggtggtggtggcgcttcttttggtattatcttgtcttggaagttgaagttggttaccgttccatctgttgttactgttttcaccgttccaaaaaccttggaagatggtgctacaaagatcttgtacaaatggcaacaagttgccgataagatcgatgatgatttgttcatgagagtcgttatcaacgtcgttgacaaaaaggataagaagggtgaaagaactgttcaaaccgcttacaactctttgttcttgggtaatgttgacaggttgttgcagatcatgaatgaatcttttccagagttgggtttgacccaaaaagattgcactgaaatgtcctggatcgagtccatcttgtatattgctggtttcccaacaaaaaccccaccagaaattttgttgcaaggtaagccaactttcaagaactacttcaaggctaagtccgacttcatcaaagaacctattccagaaactggtctggatggtatttggaaaagactattggaagaggactccccattgatgatatggaatccatacggtggtatgatggccaacatttctgaatctgaaactccatttccacacagaaagggtgttatcttcaagatccaatacttgactttgtggaccgaaccagatcaagaattggctacaaaacatgttgactggatcagaaagctgtactcttacatgacttcttacgcttctatgtttccaagagaagcctacgttaactacagagatttggacttgggtatgaacaagaatggcaactctaactcctcattcgttcaagctacagtttggggtaacaagtacttcaacaacaatttcaacaggctggtcgaaatcaagactaaggttgatccagagaatttcttcaagttcgaacaatccattccaaccttgccattgactaagaaaagaaagggtaagaagatcaaccattggtcacatccacaatttgaaaag

SEQ ID NO: 28 - DNA, 1866 bp, synthetic construct
  misc_feature 1..1866: note = CBS17-A0A1J6KPK0-full alpha factor - no signal peptide-strep Tag
  source 1..1866: mol_type = other DNA
  CDS 1..1866
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatgttgtctgataccactccagaaaagttctaccaatgtatctgcaagaactccgatttctctgatccattttctaaggctttcttcactccaaacaccgattctttcaacaccgttttaaactctaccgctcaaaacttgagatgtttgatgccaactgttccaaagccagttttgatttttactccaatggccgaatctcatgttcaagctgctgttatttgtgccaagcaattgaacttgcaattgagagttagatccggtggtcatgattatgaaggtttgtcctacatctccgttatggattctccattcgttatcgttgacttgtccaagttgagagagattgacgttaacatcgaagaaaattctgcttgggctcaagctggtgctactgttggtgaagtttattacagaatctccgagaagtctaagactcatggttttccagctggtttgtgtacctctttaggtattggtggtcatatcactggtggtgcttatggttctatgatgagaaaatttggtttgggtgccgataacgtcattgatgctagaatcgttaatgccaacggtactatcttggacagattgtctatgggtgaagatttgttctgggctattagaggtggtggtggcgcttcttttggtattatcttgtcttggaagttgaagttggttaccgttccatctgttgttactgttttcaccgttccaaaaaccttggaagatggtgctacaaagatcttgtacaaatggcaacaagttgccgataagatcgatgatgatttgttcatgagagtcgttatcaacgtcgttgacaaaaaggataagaagggtgaaagaactgttcaaaccgcttacaactctttgttcttgggtaatgttgacaggttgttgcagatcatgaatgaatcttttccagagttgggtttgacccaaaaagattgcactgaaatgtcctggatcgagtccatcttgtatattgctggtttcccaacaaaaaccccaccagaaattttgttgcaaggtaagccaactttcaagaactacttcaaggctaagtccgacttcatcaaagaacctattccagaaactggtctggatggtatttggaaaagactattggaagaggactccccattgatgatatggaatccatacggtggtatgatggccaacatttctgaatctgaaactccatttccacacagaaagggtgttatcttcaagatccaatacttgactttgtggaccgaaccagatcaagaattggctacaaaacatgttgactggatcagaaagctgtactcttacatgacttcttacgcttctatgtttccaagagaagcctacgttaactacagagatttggacttgggtatgaacaagaatggcaactctaactcctcattcgttcaagctacagtttggggtaacaagtacttcaacaacaatttcaacaggctggtcgaaatcaagactaaggttgatccagagaatttcttcaagttcgaacaatccattccaaccttgccattgactaagaaaagaaagggtaagaagatcaaccattggtcacatccacaatttgaaaag

SEQ ID NO: 29 - protein, 622 aa, synthetic construct
  REGION 1..622: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAMLSDTTPEKFYQCICKNSDFSDPFSKAFFTPNTDSFNTVLNSTAQNLRCLMPTVPKPVLIFTPMAESHVQAAVICAKQLNLQLRVRSGGHDYEGLSYISVMDSPFVIVDLSKLREIDVNIEENSAWAQAGATVGEVYYRISEKSKTHGFPAGLCTSLGIGGHITGGAYGSMMRKFGLGADNVIDARIVNANGTILDRLSMGEDLFWAIRGGGGASFGIILSWKLKLVTVPSVVTVFTVPKTLEDGATKILYKWQQVADKIDDDLFMRVVINVVDKKDKKGERTVQTAYNSLFLGNVDRLLQIMNESFPELGLTQKDCTEMSWIESILYIAGFPTKTPPEILLQGKPTFKNYFKAKSDFIKEPIPETGLDGIWKRLLEEDSPLMIWNPYGGMMANISESETPFPHRKGVIFKIQYLTLWTEPDQELATKHVDWIRKLYSYMTSYASMFPREAYVNYRDLDLGMNKNGNSNSSFVQATVWGNKYFNNNFNRLVEIKTKVDPENFFKFEQSIPTLPLTKKRKGKKINHWSHPQFEK

SEQ ID NO: 30 - DNA, 1521 bp, synthetic construct
  misc_feature 1..1521: note = CBS35-M5X864
  source 1..1521: mol_type = other DNA
atgcagatcattatcaccaagagatcctctgcttacttctctgctgttcaatcctctatccaaaacttgatcttcgttaccagaggtacttctagaccagaagctattattaccttgttgcatgattctcacggtgtcttgattagaatcagatctagaggtcatgactacgaaggtcaatcttatgttgctagagccccattcgttatcatcgatttgtttaacttgaggtccatcgacgttgacattgaaaatggttctgcttgggttgaatctggtgctactttgggtgagttgtattacagaattgccgaaaagtctaaggcttacggttttccagctggttcttgtccaactattggtgttggtggtcatatttctggtggtggttttggtactttgttcagaaagtatggtttggctgctgataacgttttggatgctaaaatcgttgatgtcaacggtagagttttggacagaaaacctatgggtgaagaattattctgggctattagaggtggtggtggatcttcttttggtgttattttggcttggaagttgaagttggttccagttccaccatctgttactagacatgaagatttgttcttgcactccgttataggtgttggtaacaaagctggtactaatggtgataagaccattattatcgagttcagctccttgtttttgggtccagttgaaaagttgttgccatccatgcaagataacttctccgaattgtctgttgatagatccgattgcactgaaatgtcctggattgaatctgtcttgtacttcgctttcatcccaagaaatgaatctgaagccttgttgggtagaacccaacaatctaaatctttcgctaagccacaaggtattttctggactcatagaactccaaccatgaacgttaattctccaacctctaagttctgtactacttgtactccatctaagacctacaagagaggtacatctccaaagattgaagttactggtactttcggcactttgaaaacttctacctctgttttcaaggccaagtccgattatgttaacgaacctatttctaaggctggtttggaaggtttgtggcaaactttgatagaagttaaggcctacctgatcttgacattatacggtggtaagatctccgagatttccaactctgaaattcatttcccacacaggtctggtaacttgttcaaaattcagtacatggttacctgggacgactacagagaaaacgaaattggtgaatccctgatgagaaagttgtacgattatatggctccctacgtttctaaatctccaagagctgcttatctgaactacaaggatttggacttaggtagaaacaacgatgttcatacttcttacgctcaagctagtatttggggcttgaagtactttaagaacaacttcagaagattggtccacgttaagactttggttgatccaggtaatttcttcagggacgaacaatctattccatccgttagatcttcccgtaagaag

SEQ ID NO: 31 - DNA, 1545 bp, synthetic construct
  misc_feature 1..1545: note = CBS35-M5X864-strep Tag
  source 1..1545: mol_type = other DNA
  CDS 1..1545
atgcagatcattatcaccaagagatcctctgcttacttctctgctgttcaatcctctatccaaaacttgatcttcgttaccagaggtacttctagaccagaagctattattaccttgttgcatgattctcacggtgtcttgattagaatcagatctagaggtcatgactacgaaggtcaatcttatgttgctagagccccattcgttatcatcgatttgtttaacttgaggtccatcgacgttgacattgaaaatggttctgcttgggttgaatctggtgctactttgggtgagttgtattacagaattgccgaaaagtctaaggcttacggttttccagctggttcttgtccaactattggtgttggtggtcatatttctggtggtggttttggtactttgttcagaaagtatggtttggctgctgataacgttttggatgctaaaatcgttgatgtcaacggtagagttttggacagaaaacctatgggtgaagaattattctgggctattagaggtggtggtggatcttcttttggtgttattttggcttggaagttgaagttggttccagttccaccatctgttactagacatgaagatttgttcttgcactccgttataggtgttggtaacaaagctggtactaatggtgataagaccattattatcgagttcagctccttgtttttgggtccagttgaaaagttgttgccatccatgcaagataacttctccgaattgtctgttgatagatccgattgcactgaaatgtcctggattgaatctgtcttgtacttcgctttcatcccaagaaatgaatctgaagccttgttgggtagaacccaacaatctaaatctttcgctaagccacaaggtattttctggactcatagaactccaaccatgaacgttaattctccaacctctaagttctgtactacttgtactccatctaagacctacaagagaggtacatctccaaagattgaagttactggtactttcggcactttgaaaacttctacctctgttttcaaggccaagtccgattatgttaacgaacctatttctaaggctggtttggaaggtttgtggcaaactttgatagaagttaaggcctacctgatcttgacattatacggtggtaagatctccgagatttccaactctgaaattcatttcccacacaggtctggtaacttgttcaaaattcagtacatggttacctgggacgactacagagaaaacgaaattggtgaatccctgatgagaaagttgtacgattatatggctccctacgtttctaaatctccaagagctgcttatctgaactacaaggatttggacttaggtagaaacaacgatgttcatacttcttacgctcaagctagtatttggggcttgaagtactttaagaacaacttcagaagattggtccacgttaagactttggttgatccaggtaatttcttcagggacgaacaatctattccatccgttagatcttcccgtaagaagtggtcacatccacaatttgaaaag

SEQ ID NO: 32 - protein, 515 aa, synthetic construct
  REGION 1..515: note = Synthetic Construct
MQIIITKRSSAYFSAVQSSIQNLIFVTRGTSRPEAIITLLHDSHGVLIRIRSRGHDYEGQSYVARAPFVIIDLFNLRSIDVDIENGSAWVESGATLGELYYRIAEKSKAYGFPAGSCPTIGVGGHISGGGFGTLFRKYGLAADNVLDAKIVDVNGRVLDRKPMGEELFWAIRGGGGSSFGVILAWKLKLVPVPPSVTRHEDLFLHSVIGVGNKAGTNGDKTIIIEFSSLFLGPVEKLLPSMQDNFSELSVDRSDCTEMSWIESVLYFAFIPRNESEALLGRTQQSKSFAKPQGIFWTHRTPTMNVNSPTSKFCTTCTPSKTYKRGTSPKIEVTGTFGTLKTSTSVFKAKSDYVNEPISKAGLEGLWQTLIEVKAYLILTLYGGKISEISNSEIHFPHRSGNLFKIQYMVTWDDYRENEIGESLMRKLYDYMAPYVSKSPRAAYLNYKDLDLGRNNDVHTSYAQASIWGLKYFKNNFRRLVHVKTLVDPGNFFRDEQSIPSVRSSRKKWSHPQFEK

SEQ ID NO: 33 - DNA, 1602 bp, synthetic construct
  misc_feature 1..1602: note = CBS35-M5X864- truncated alpha factor - strep Tag
  source 1..1602: mol_type = other DNA
  CDS 1..1602
atgagatttccttccatcttcacggctgtgctatttgcagcatcctccgcacttgcaatgcagatcattatcaccaagagatcctctgcttacttctctgctgttcaatcctctatccaaaacttgatcttcgttaccagaggtacttctagaccagaagctattattaccttgttgcatgattctcacggtgtcttgattagaatcagatctagaggtcatgactacgaaggtcaatcttatgttgctagagccccattcgttatcatcgatttgtttaacttgaggtccatcgacgttgacattgaaaatggttctgcttgggttgaatctggtgctactttgggtgagttgtattacagaattgccgaaaagtctaaggcttacggttttccagctggttcttgtccaactattggtgttggtggtcatatttctggtggtggttttggtactttgttcagaaagtatggtttggctgctgataacgttttggatgctaaaatcgttgatgtcaacggtagagttttggacagaaaacctatgggtgaagaattattctgggctattagaggtggtggtggatcttcttttggtgttattttggcttggaagttgaagttggttccagttccaccatctgttactagacatgaagatttgttcttgcactccgttataggtgttggtaacaaagctggtactaatggtgataagaccattattatcgagttcagctccttgtttttgggtccagttgaaaagttgttgccatccatgcaagataacttctccgaattgtctgttgatagatccgattgcactgaaatgtcctggattgaatctgtcttgtacttcgctttcatcccaagaaatgaatctgaagccttgttgggtagaacccaacaatctaaatctttcgctaagccacaaggtattttctggactcatagaactccaaccatgaacgttaattctccaacctctaagttctgtactacttgtactccatctaagacctacaagagaggtacatctccaaagattgaagttactggtactttcggcactttgaaaacttctacctctgttttcaaggccaagtccgattatgttaacgaacctatttctaaggctggtttggaaggtttgtggcaaactttgatagaagttaaggcctacctgatcttgacattatacggtggtaagatctccgagatttccaactctgaaattcatttcccacacaggtctggtaacttgttcaaaattcagtacatggttacctgggacgactacagagaaaacgaaattggtgaatccctgatgagaaagttgtacgattatatggctccctacgtttctaaatctccaagagctgcttatctgaactacaaggatttggacttaggtagaaacaacgatgttcatacttcttacgctcaagctagtatttggggcttgaagtactttaagaacaacttcagaagattggtccacgttaagactttggttgatccaggtaatttcttcagggacgaacaatctattccatccgttagatcttcccgtaagaagtggtcacatccacaatttgaaaag

SEQ ID NO: 34 - protein, 534 aa, synthetic construct
  REGION 1..534: note = Synthetic Construct
MRFPSIFTAVLFAASSALAMQIIITKRSSAYFSAVQSSIQNLIFVTRGTSRPEAIITLLHDSHGVLIRIRSRGHDYEGQSYVARAPFVIIDLFNLRSIDVDIENGSAWVESGATLGELYYRIAEKSKAYGFPAGSCPTIGVGGHISGGGFGTLFRKYGLAADNVLDAKIVDVNGRVLDRKPMGEELFWAIRGGGGSSFGVILAWKLKLVPVPPSVTRHEDLFLHSVIGVGNKAGTNGDKTIIIEFSSLFLGPVEKLLPSMQDNFSELSVDRSDCTEMSWIESVLYFAFIPRNESEALLGRTQQSKSFAKPQGIFWTHRTPTMNVNSPTSKFCTTCTPSKTYKRGTSPKIEVTGTFGTLKTSTSVFKAKSDYVNEPISKAGLEGLWQTLIEVKAYLILTLYGGKISEISNSEIHFPHRSGNLFKIQYMVTWDDYRENEIGESLMRKLYDYMAPYVSKSPRAAYLNYKDLDLGRNNDVHTSYAQASIWGLKYFKNNFRRLVHVKTLVDPGNFFRDEQSIPSVRSSRKKWSHPQFEK

SEQ ID NO: 35 - DNA, 1812 bp, synthetic construct
  misc_feature 1..1812: note = CBS35-M5X864- full alpha factor - strep Tag
  source 1..1812: mol_type = other DNA
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatgcagatcattatcaccaagagatcctctgcttacttctctgctgttcaatcctctatccaaaacttgatcttcgttaccagaggtacttctagaccagaagctattattaccttgttgcatgattctcacggtgtcttgattagaatcagatctagaggtcatgactacgaaggtcaatcttatgttgctagagccccattcgttatcatcgatttgtttaacttgaggtccatcgacgttgacattgaaaatggttctgcttgggttgaatctggtgctactttgggtgagttgtattacagaattgccgaaaagtctaaggcttacggttttccagctggttcttgtccaactattggtgttggtggtcatatttctggtggtggttttggtactttgttcagaaagtatggtttggctgctgataacgttttggatgctaaaatcgttgatgtcaacggtagagttttggacagaaaacctatgggtgaagaattattctgggctattagaggtggtggtggatcttcttttggtgttattttggcttggaagttgaagttggttccagttccaccatctgttactagacatgaagatttgttcttgcactccgttataggtgttggtaacaaagctggtactaatggtgataagaccattattatcgagttcagctccttgtttttgggtccagttgaaaagttgttgccatccatgcaagataacttctccgaattgtctgttgatagatccgattgcactgaaatgtcctggattgaatctgtcttgtacttcgctttcatcccaagaaatgaatctgaagccttgttgggtagaacccaacaatctaaatctttcgctaagccacaaggtattttctggactcatagaactccaaccatgaacgttaattctccaacctctaagttctgtactacttgtactccatctaagacctacaagagaggtacatctccaaagattgaagttactggtactttcggcactttgaaaacttctacctctgttttcaaggccaagtccgattatgttaacgaacctatttctaaggctggtttggaaggtttgtggcaaactttgatagaagttaaggcctacctgatcttgacattatacggtggtaagatctccgagatttccaactctgaaattcatttcccacacaggtctggtaacttgttcaaaattcagtacatggttacctgggacgactacagagaaaacgaaattggtgaatccctgatgagaaagttgtacgattatatggctccctacgtttctaaatctccaagagctgcttatctgaactacaaggatttggacttaggtagaaacaacgatgttcatacttcttacgctcaagctagtatttggggcttgaagtactttaagaacaacttcagaagattggtccacgttaagactttggttgatccaggtaatttcttcagggacgaacaatctattccatccgttagatcttcccgtaagaagtggtcacatccacaatttgaaaag

SEQ ID NO: 36 - DNA, 1620 bp, synthetic construct
  misc_feature 1..1620: note = CBS59-O64745
  source 1..1620: mol_type = other DNA
  CDS 1..1620
atgaagttctggtcaagacctttgaccttcttgatcatcatcatctacctgatcatccaacaggttaattcttctccaccatccttgtctattccagaacatttcttgagatgcttggatacccaaccatctgatcatggttctccaaattctagaactgccgttattccaacgaactcttctttttctaccaacttgatgaacggcgtcagaaatttgagattcgcttctgtttctactaggaagccagaagttatagttgctgctgttactgaaacccatattagagctaccatttcttgctgcaagttgttgaacttggaattgagaattagatccggtggtcatgattacgaaggtttctcttatacttctccagttccattcgttatcctggacatgtacaacttcaacaagatcgacattaacatgaaggacgaaaccgtctggattcaatctggtgcttctttgggtcagttgtactacaatattgcctccaagtctaaggttcatgcttttccagctggtgtttgtccaaaagttggtgctggtggtcatttttctggtggtggttttggtaatttgatgaggaaatacggtttgtccatcgatcatatcattgacgcccaaattatggatgctaacggtaaggtttacagaaacagacaagctatgggtgaagatgttttctgggctattagaggtggtggtggcggttcttatggtgttattttggcttggaagattaagttggttagggttccagaaaaggtcaccgtttttaagttggaaagaaccgttagagaaggtgccgttgatttggttcataagtggcaacaagttgccccagttattgatagagatttgttcatcagattggagatcaagcccatcaacagaaagatttctaagggtaagaccatcaaggtgtccttcattggtatgtttttgggtttgccagaacgtttgttgaacatcactaagcaatctttcccagaactgcacttgacaaaagaagattgcatggttaagaagtggatcgagtcatctgttttttgggcaaattacccagaaaaagccccaattgagttgttgttgaaaagagtctctaccaacgagtactactggaagagaacttctgattttgttcaggccccaatttctaaacaaggtttggctaagattttccagaccatgattgatcattctccattgcctagaagagtttggatgcaatggaatccttggggtggtaaaatggctgaaattgcttctgatgctactgcctttgttcacagaggtggtaatgttttcatgatcgaacatttcatgaactggtacagaccaggtgatgaattggaagaaaagttcttggctattgccaggtctttcaaagaagctatggctccatttgtttctaagaatccaagagaagccttcttcaactacagagatgttgatattggtatcactaccccaggttacaatgctacttatgaaggtgctaaagtttacggcgattcttactttaagggtaactacttgaggttggttaagatcaaggctagattcgacagaacgaacttcttcagatctcaacaaggtattccagttttggcc

SEQ ID NO: 37 - protein, 540 aa, synthetic construct
  REGION 1..540: note = Synthetic Construct
MKFWSRPLTFLIIIIYLIIQQVNSSPPSLSIPEHFLRCLDTQPSDHGSPNSRTAVIPTNSSFSTNLMNGVRNLRFASVSTRKPEVIVAAVTETHIRATISCCKLLNLELRIRSGGHDYEGFSYTSPVPFVILDMYNFNKIDINMKDETVWIQSGASLGQLYYNIASKSKVHAFPAGVCPKVGAGGHFSGGGFGNLMRKYGLSIDHIIDAQIMDANGKVYRNRQAMGEDVFWAIRGGGGGSYGVILAWKIKLVRVPEKVTVFKLERTVREGAVDLVHKWQQVAPVIDRDLFIRLEIKPINRKISKGKTIKVSFIGMFLGLPERLLNITKQSFPELHLTKEDCMVKKWIESSVFWANYPEKAPIELLLKRVSTNEYYWKRTSDFVQAPISKQGLAKIFQTMIDHSPLPRRVWMQWNPWGGKMAEIASDATAFVHRGGNVFMIEHFMNWYRPGDELEEKFLAIARSFKEAMAPFVSKNPREAFFNYRDVDIGITTPGYNATYEGAKVYGDSYFKGNYLRLVKIKARFDRTNFFRSQQGIPVLA

SEQ ID NO: 38 - DNA, 1551 bp, synthetic construct
  misc_feature 1..1551: note = CBS59-O64745-no signal peptide
  source 1..1551: mol_type = other DNA
  CDS 1..1551
atgtctccaccatccttgtctattccagaacatttcttgagatgcttggatacccaaccatctgatcatggttctccaaattctagaactgccgttattccaacgaactcttctttttctaccaacttgatgaacggcgtcagaaatttgagattcgcttctgtttctactaggaagccagaagttatagttgctgctgttactgaaacccatattagagctaccatttcttgctgcaagttgttgaacttggaattgagaattagatccggtggtcatgattacgaaggtttctcttatacttctccagttccattcgttatcctggacatgtacaacttcaacaagatcgacattaacatgaaggacgaaaccgtctggattcaatctggtgcttctttgggtcagttgtactacaatattgcctccaagtctaaggttcatgcttttccagctggtgtttgtccaaaagttggtgctggtggtcatttttctggtggtggttttggtaatttgatgaggaaatacggtttgtccatcgatcatatcattgacgcccaaattatggatgctaacggtaaggtttacagaaacagacaagctatgggtgaagatgttttctgggctattagaggtggtggtggcggttcttatggtgttattttggcttggaagattaagttggttagggttccagaaaaggtcaccgtttttaagttggaaagaaccgttagagaaggtgccgttgatttggttcataagtggcaacaagttgccccagttattgatagagatttgttcatcagattggagatcaagcccatcaacagaaagatttctaagggtaagaccatcaaggtgtccttcattggtatgtttttgggtttgccagaacgtttgttgaacatcactaagcaatctttcccagaactgcacttgacaaaagaagattgcatggttaagaagtggatcgagtcatctgttttttgggcaaattacccagaaaaagccccaattgagttgttgttgaaaagagtctctaccaacgagtactactggaagagaacttctgattttgttcaggccccaatttctaaacaaggtttggctaagattttccagaccatgattgatcattctccattgcctagaagagtttggatgcaatggaatccttggggtggtaaaatggctgaaattgcttctgatgctactgcctttgttcacagaggtggtaatgttttcatgatcgaacatttcatgaactggtacagaccaggtgatgaattggaagaaaagttcttggctattgccaggtctttcaaagaagctatggctccatttgtttctaagaatccaagagaagccttcttcaactacagagatgttgatattggtatcactaccccaggttacaatgctacttatgaaggtgctaaagtttacggcgattcttactttaagggtaactacttgaggttggttaagatcaaggctagattcgacagaacgaacttcttcagatctcaacaaggtattccagttttggcc

SEQ ID NO: 39 - protein, 517 aa, synthetic construct
  REGION 1..517: note = Synthetic Construct
MSPPSLSIPEHFLRCLDTQPSDHGSPNSRTAVIPTNSSFSTNLMNGVRNLRFASVSTRKPEVIVAAVTETHIRATISCCKLLNLELRIRSGGHDYEGFSYTSPVPFVILDMYNFNKIDINMKDETVWIQSGASLGQLYYNIASKSKVHAFPAGVCPKVGAGGHFSGGGFGNLMRKYGLSIDHIIDAQIMDANGKVYRNRQAMGEDVFWAIRGGGGGSYGVILAWKIKLVRVPEKVTVFKLERTVREGAVDLVHKWQQVAPVIDRDLFIRLEIKPINRKISKGKTIKVSFIGMFLGLPERLLNITKQSFPELHLTKEDCMVKKWIESSVFWANYPEKAPIELLLKRVSTNEYYWKRTSDFVQAPISKQGLAKIFQTMIDHSPLPRRVWMQWNPWGGKMAEIASDATAFVHRGGNVFMIEHFMNWYRPGDELEEKFLAIARSFKEAMAPFVSKNPREAFFNYRDVDIGITTPGYNATYEGAKVYGDSYFKGNYLRLVKIKARFDRTNFFRSQQGIPVLA

SEQ ID NO: 40 - DNA, 1575 bp, synthetic construct
  misc_feature 1..1575: note = CBS59-O64745-no signal peptide-strep Tag
  source 1..1575: mol_type = other DNA
  CDS 1..1575
atgtctccaccatccttgtctattccagaacatttcttgagatgcttggatacccaaccatctgatcatggttctccaaattctagaactgccgttattccaacgaactcttctttttctaccaacttgatgaacggcgtcagaaatttgagattcgcttctgtttctactaggaagccagaagttatagttgctgctgttactgaaacccatattagagctaccatttcttgctgcaagttgttgaacttggaattgagaattagatccggtggtcatgattacgaaggtttctcttatacttctccagttccattcgttatcctggacatgtacaacttcaacaagatcgacattaacatgaaggacgaaaccgtctggattcaatctggtgcttctttgggtcagttgtactacaatattgcctccaagtctaaggttcatgcttttccagctggtgtttgtccaaaagttggtgctggtggtcatttttctggtggtggttttggtaatttgatgaggaaatacggtttgtccatcgatcatatcattgacgcccaaattatggatgctaacggtaaggtttacagaaacagacaagctatgggtgaagatgttttctgggctattagaggtggtggtggcggttcttatggtgttattttggcttggaagattaagttggttagggttccagaaaaggtcaccgtttttaagttggaaagaaccgttagagaaggtgccgttgatttggttcataagtggcaacaagttgccccagttattgatagagatttgttcatcagattggagatcaagcccatcaacagaaagatttctaagggtaagaccatcaaggtgtccttcattggtatgtttttgggtttgccagaacgtttgttgaacatcactaagcaatctttcccagaactgcacttgacaaaagaagattgcatggttaagaagtggatcgagtcatctgttttttgggcaaattacccagaaaaagccccaattgagttgttgttgaaaagagtctctaccaacgagtactactggaagagaacttctgattttgttcaggccccaatttctaaacaaggtttggctaagattttccagaccatgattgatcattctccattgcctagaagagtttggatgcaatggaatccttggggtggtaaaatggctgaaattgcttctgatgctactgcctttgttcacagaggtggtaatgttttcatgatcgaacatttcatgaactggtacagaccaggtgatgaattggaagaaaagttcttggctattgccaggtctttcaaagaagctatggctccatttgtttctaagaatccaagagaagccttcttcaactacagagatgttgatattggtatcactaccccaggttacaatgctacttatgaaggtgctaaagtttacggcgattcttactttaagggtaactacttgaggttggttaagatcaaggctagattcgacagaacgaacttcttcagatctcaacaaggtattccagttttggcctggtcacatccacaatttgaaaag

SEQ ID NO: 41 - protein, 525 aa, synthetic construct
  REGION 1..525: note = Synthetic Construct
MSPPSLSIPEHFLRCLDTQPSDHGSPNSRTAVIPTNSSFSTNLMNGVRNLRFASVSTRKPEVIVAAVTETHIRATISCCKLLNLELRIRSGGHDYEGFSYTSPVPFVILDMYNFNKIDINMKDETVWIQSGASLGQLYYNIASKSKVHAFPAGVCPKVGAGGHFSGGGFGNLMRKYGLSIDHIIDAQIMDANGKVYRNRQAMGEDVFWAIRGGGGGSYGVILAWKIKLVRVPEKVTVFKLERTVREGAVDLVHKWQQVAPVIDRDLFIRLEIKPINRKISKGKTIKVSFIGMFLGLPERLLNITKQSFPELHLTKEDCMVKKWIESSVFWANYPEKAPIELLLKRVSTNEYYWKRTSDFVQAPISKQGLAKIFQTMIDHSPLPRRVWMQWNPWGGKMAEIASDATAFVHRGGNVFMIEHFMNWYRPGDELEEKFLAIARSFKEAMAPFVSKNPREAFFNYRDVDIGITTPGYNATYEGAKVYGDSYFKGNYLRLVKIKARFDRTNFFRSQQGIPVLAWSHPQFEK

SEQ ID NO: 42 - DNA, 1632 bp, synthetic construct
  misc_feature 1..1632: note = CBS59-O64745- truncated alpha factor -no signal peptide-strep Tag
  source 1..1632: mol_type = other DNA
  CDS 1..1632
atgagatttccttccatcttcacggctgtgctatttgcagcatcctccgcacttgcaatgtctccaccatccttgtctattccagaacatttcttgagatgcttggatacccaaccatctgatcatggttctccaaattctagaactgccgttattccaacgaactcttctttttctaccaacttgatgaacggcgtcagaaatttgagattcgcttctgtttctactaggaagccagaagttatagttgctgctgttactgaaacccatattagagctaccatttcttgctgcaagttgttgaacttggaattgagaattagatccggtggtcatgattacgaaggtttctcttatacttctccagttccattcgttatcctggacatgtacaacttcaacaagatcgacattaacatgaaggacgaaaccgtctggattcaatctggtgcttctttgggtcagttgtactacaatattgcctccaagtctaaggttcatgcttttccagctggtgtttgtccaaaagttggtgctggtggtcatttttctggtggtggttttggtaatttgatgaggaaatacggtttgtccatcgatcatatcattgacgcccaaattatggatgctaacggtaaggtttacagaaacagacaagctatgggtgaagatgttttctgggctattagaggtggtggtggcggttcttatggtgttattttggcttggaagattaagttggttagggttccagaaaaggtcaccgtttttaagttggaaagaaccgttagagaaggtgccgttgatttggttcataagtggcaacaagttgccccagttattgatagagatttgttcatcagattggagatcaagcccatcaacagaaagatttctaagggtaagaccatcaaggtgtccttcattggtatgtttttgggtttgccagaacgtttgttgaacatcactaagcaatctttcccagaactgcacttgacaaaagaagattgcatggttaagaagtggatcgagtcatctgttttttgggcaaattacccagaaaaagccccaattgagttgttgttgaaaagagtctctaccaacgagtactactggaagagaacttctgattttgttcaggccccaatttctaaacaaggtttggctaagattttccagaccatgattgatcattctccattgcctagaagagtttggatgcaatggaatccttggggtggtaaaatggctgaaattgcttctgatgctactgcctttgttcacagaggtggtaatgttttcatgatcgaacatttcatgaactggtacagaccaggtgatgaattggaagaaaagttcttggctattgccaggtctttcaaagaagctatggctccatttgtttctaagaatccaagagaagccttcttcaactacagagatgttgatattggtatcactaccccaggttacaatgctacttatgaaggtgctaaagtttacggcgattcttactttaagggtaactacttgaggttggttaagatcaaggctagattcgacagaacgaacttcttcagatctcaacaaggtattccagttttggcctggtcacatccacaatttgaaaag

SEQ ID NO: 43 - protein, 544 aa, synthetic construct
  REGION 1..544: note = Synthetic Construct
MRFPSIFTAVLFAASSALAMSPPSLSIPEHFLRCLDTQPSDHGSPNSRTAVIPTNSSFSTNLMNGVRNLRFASVSTRKPEVIVAAVTETHIRATISCCKLLNLELRIRSGGHDYEGFSYTSPVPFVILDMYNFNKIDINMKDETVWIQSGASLGQLYYNIASKSKVHAFPAGVCPKVGAGGHFSGGGFGNLMRKYGLSIDHIIDAQIMDANGKVYRNRQAMGEDVFWAIRGGGGGSYGVILAWKIKLVRVPEKVTVFKLERTVREGAVDLVHKWQQVAPVIDRDLFIRLEIKPINRKISKGKTIKVSFIGMFLGLPERLLNITKQSFPELHLTKEDCMVKKWIESSVFWANYPEKAPIELLLKRVSTNEYYWKRTSDFVQAPISKQGLAKIFQTMIDHSPLPRRVWMQWNPWGGKMAEIASDATAFVHRGGNVFMIEHFMNWYRPGDELEEKFLAIARSFKEAMAPFVSKNPREAFFNYRDVDIGITTPGYNATYEGAKVYGDSYFKGNYLRLVKIKARFDRTNFFRSQQGIPVLAWSHPQFEK

SEQ ID NO: 44 - DNA, 1842 bp, synthetic construct
  misc_feature 1..1842: note = CBS59-O64745-full alpha factor-no signal peptide-strep Tag
  source 1..1842: mol_type = other DNA
  CDS 1..1842
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatgtctccaccatccttgtctattccagaacatttcttgagatgcttggatacccaaccatctgatcatggttctccaaattctagaactgccgttattccaacgaactcttctttttctaccaacttgatgaacggcgtcagaaatttgagattcgcttctgtttctactaggaagccagaagttatagttgctgctgttactgaaacccatattagagctaccatttcttgctgcaagttgttgaacttggaattgagaattagatccggtggtcatgattacgaaggtttctcttatacttctccagttccattcgttatcctggacatgtacaacttcaacaagatcgacattaacatgaaggacgaaaccgtctggattcaatctggtgcttctttgggtcagttgtactacaatattgcctccaagtctaaggttcatgcttttccagctggtgtttgtccaaaagttggtgctggtggtcatttttctggtggtggttttggtaatttgatgaggaaatacggtttgtccatcgatcatatcattgacgcccaaattatggatgctaacggtaaggtttacagaaacagacaagctatgggtgaagatgttttctgggctattagaggtggtggtggcggttcttatggtgttattttggcttggaagattaagttggttagggttccagaaaaggtcaccgtttttaagttggaaagaaccgttagagaaggtgccgttgatttggttcataagtggcaacaagttgccccagttattgatagagatttgttcatcagattggagatcaagcccatcaacagaaagatttctaagggtaagaccatcaaggtgtccttcattggtatgtttttgggtttgccagaacgtttgttgaacatcactaagcaatctttcccagaactgcacttgacaaaagaagattgcatggttaagaagtggatcgagtcatctgttttttgggcaaattacccagaaaaagccccaattgagttgttgttgaaaagagtctctaccaacgagtactactggaagagaacttctgattttgttcaggccccaatttctaaacaaggtttggctaagattttccagaccatgattgatcattctccattgcctagaagagtttggatgcaatggaatccttggggtggtaaaatggctgaaattgcttctgatgctactgcctttgttcacagaggtggtaatgttttcatgatcgaacatttcatgaactggtacagaccaggtgatgaattggaagaaaagttcttggctattgccaggtctttcaaagaagctatggctccatttgtttctaagaatccaagagaagccttcttcaactacagagatgttgatattggtatcactaccccaggttacaatgctacttatgaaggtgctaaagtttacggcgattcttactttaagggtaactacttgaggttggttaagatcaaggctagattcgacagaacgaacttcttcagatctcaacaaggtattccagttttggcctggtcacatccacaatttgaaaag

SEQ ID NO: 45 - protein, 614 aa, synthetic construct
  REGION 1..614: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAMSPPSLSIPEHFLRCLDTQPSDHGSPNSRTAVIPTNSSFSTNLMNGVRNLRFASVSTRKPEVIVAAVTETHIRATISCCKLLNLELRIRSGGHDYEGFSYTSPVPFVILDMYNFNKIDINMKDETVWIQSGASLGQLYYNIASKSKVHAFPAGVCPKVGAGGHFSGGGFGNLMRKYGLSIDHIIDAQIMDANGKVYRNRQAMGEDVFWAIRGGGGGSYGVILAWKIKLVRVPEKVTVFKLERTVREGAVDLVHKWQQVAPVIDRDLFIRLEIKPINRKISKGKTIKVSFIGMFLGLPERLLNITKQSFPELHLTKEDCMVKKWIESSVFWANYPEKAPIELLLKRVSTNEYYWKRTSDFVQAPISKQGLAKIFQTMIDHSPLPRRVWMQWNPWGGKMAEIASDATAFVHRGGNVFMIEHFMNWYRPGDELEEKFLAIARSFKEAMAPFVSKNPREAFFNYRDVDIGITTPGYNATYEGAKVYGDSYFKGNYLRLVKIKARFDRTNFFRSQQGIPVLAWSHPQFEK

SEQ ID NO: 46 - DNA, 1605 bp, synthetic construct
  misc_feature 1..1605: note = CBS69-P93479
  source 1..1605: mol_type = other DNA
  CDS 1..1605
atgatgtgcagatctttgaccttgaggttcttcttgttcatcgttttgttgcaaacctgtgttagaggtggtgatgttaacgataacttgttgtcctcttgcttgaactctcatggtgttcataacttcactactttgtctaccgataccaactccgattacttcaagttgttgcatgcttctatgcaaaaccctttgtttgctaagccaaccgtttctaagccatcttttatcgttatgccaggctccaaagaagaattgtcatctactgttcattgctgcactagagaatcttggaccattagattgagatctggtggtcattcttacgagggtttgtcttatactgctgatactccattcgttatcgtcgacatgatgaacttgaacagaatctccatcgatgtcttgtctgaaactgcttgggttgaatctggtgctactttgggtgaactgtattatgctattgctcaatccactgataccttgggttttactgctggttggtgtccaactgttggttcaggtggtcatatttctggtggtggttttggtatgatgtctagaaagtatggtttggctgctgataacgttgttgatgccattttgattgattccaacggtgctatcttggacagagaaaaaatgggtgatgatgttttctgggctattagaggcggtggtggtggcgtttggggtgcaatctatgcttggaaaatcaagttattgccagtgccagaaaagttgaccgttttcagagttactaagaacgtcggtattgaagatgcctcttctttgttgcacaaatggcaatatgttgccgatgaattggatgaggatttcaccgtttctgttttaggtggtgttaatggtaatgatgcctggttgatgttcttgggtttacatttgggtagaaaggatgctgccaagaccattattgacgaaaagtttccagaattgggcctagtcgacaaagaatttcaagaaatgtcttggggtgaatcgatggcttttttgtctggtttggataccatctctgaactgaacaacagattcttgaagttcgacgaaagagctttcaagaccaaggttgatttcaccaaagtttccgttccattgaacgtttttagacacgctttggaaatgttgtctgaacaaccaggtggtttcattgctttgaatggtttcggtggtaagatgtccgaaatttctactgactttaccccatttccacacagaaaaggtacgaagttgatgtttgagtacattattgcctggaaccaggacgaagaatctaagattggtgaattttctgaatggctggccaagttctatgattacttggaaccattcgtttccaaagaacctagagttggttacgttaaccacatcgatttggatattggtggtatcgattggaggaacaaatcttctactaccaacgctgttgaaattgctagaaactggggtgaaagatacttcagctctaactacgaaagattggttaaggccaagactttgatcgatccaaacaacgttttcaaccatccacaatctattccaccaatgatgaagtttgaagagatctacatgctgaaagaattg

SEQ ID NO: 47 - protein, 535 aa, synthetic construct
  REGION 1..535: note = Synthetic Construct
MMCRSLTLRFFLFIVLLQTCVRGGDVNDNLLSSCLNSHGVHNFTTLSTDTNSDYFKLLHASMQNPLFAKPTVSKPSFIVMPGSKEELSSTVHCCTRESWTIRLRSGGHSYEGLSYTADTPFVIVDMMNLNRISIDVLSETAWVESGATLGELYYAIAQSTDTLGFTAGWCPTVGSGGHISGGGFGMMSRKYGLAADNVVDAILIDSNGAILDREKMGDDVFWAIRGGGGGVWGAIYAWKIKLLPVPEKLTVFRVTKNVGIEDASSLLHKWQYVADELDEDFTVSVLGGVNGNDAWLMFLGLHLGRKDAAKTIIDEKFPELGLVDKEFQEMSWGESMAFLSGLDTISELNNRFLKFDERAFKTKVDFTKVSVPLNVFRHALEMLSEQPGGFIALNGFGGKMSEISTDFTPFPHRKGTKLMFEYIIAWNQDEESKIGEFSEWLAKFYDYLEPFVSKEPRVGYVNHIDLDIGGIDWRNKSSTTNAVEIARNWGERYFSSNYERLVKAKTLIDPNNVFNHPQSIPPMMKFEEIYMLKEL

SEQ ID NO: 48 - DNA, 1539 bp, synthetic construct
  misc_feature 1..1539: note = CBS69-P93479-no signal peptide
  source 1..1539: mol_type = other DNA
  CDS 1..1539
atgggtgatgttaacgataacttgttgtcctcttgcttgaactctcatggtgttcataacttcactactttgtctaccgataccaactccgattacttcaagttgttgcatgcttctatgcaaaaccctttgtttgctaagccaaccgtttctaagccatcttttatcgttatgccaggctccaaagaagaattgtcatctactgttcattgctgcactagagaatcttggaccattagattgagatctggtggtcattcttacgagggtttgtcttatactgctgatactccattcgttatcgtcgacatgatgaacttgaacagaatctccatcgatgtcttgtctgaaactgcttgggttgaatctggtgctactttgggtgaactgtattatgctattgctcaatccactgataccttgggttttactgctggttggtgtccaactgttggttcaggtggtcatatttctggtggtggttttggtatgatgtctagaaagtatggtttggctgctgataacgttgttgatgccattttgattgattccaacggtgctatcttggacagagaaaaaatgggtgatgatgttttctgggctattagaggcggtggtggtggcgtttggggtgcaatctatgcttggaaaatcaagttattgccagtgccagaaaagttgaccgttttcagagttactaagaacgtcggtattgaagatgcctcttctttgttgcacaaatggcaatatgttgccgatgaattggatgaggatttcaccgtttctgttttaggtggtgttaatggtaatgatgcctggttgatgttcttgggtttacatttgggtagaaaggatgctgccaagaccattattgacgaaaagtttccagaattgggcctagtcgacaaagaatttcaagaaatgtcttggggtgaatcgatggcttttttgtctggtttggataccatctctgaactgaacaacagattcttgaagttcgacgaaagagctttcaagaccaaggttgatttcaccaaagtttccgttccattgaacgtttttagacacgctttggaaatgttgtctgaacaaccaggtggtttcattgctttgaatggtttcggtggtaagatgtccgaaatttctactgactttaccccatttccacacagaaaaggtacgaagttgatgtttgagtacattattgcctggaaccaggacgaagaatctaagattggtgaattttctgaatggctggccaagttctatgattacttggaaccattcgtttccaaagaacctagagttggttacgttaaccacatcgatttggatattggtggtatcgattggaggaacaaatcttctactaccaacgctgttgaaattgctagaaactggggtgaaagatacttcagctctaactacgaaagattggttaaggccaagactttgatcgatccaaacaacgttttcaaccatccacaatctattccaccaatgatgaagtttgaagagatctacatgctgaaagaattg

SEQ ID NO: 49 - protein, 513 aa, synthetic construct
  REGION 1..513: note = Synthetic Construct
MGDVNDNLLSSCLNSHGVHNFTTLSTDTNSDYFKLLHASMQNPLFAKPTVSKPSFIVMPGSKEELSSTVHCCTRESWTIRLRSGGHSYEGLSYTADTPFVIVDMMNLNRISIDVLSETAWVESGATLGELYYAIAQSTDTLGFTAGWCPTVGSGGHISGGGFGMMSRKYGLAADNVVDAILIDSNGAILDREKMGDDVFWAIRGGGGGVWGAIYAWKIKLLPVPEKLTVFRVTKNVGIEDASSLLHKWQYVADELDEDFTVSVLGGVNGNDAWLMFLGLHLGRKDAAKTIIDEKFPELGLVDKEFQEMSWGESMAFLSGLDTISELNNRFLKFDERAFKTKVDFTKVSVPLNVFRHALEMLSEQPGGFIALNGFGGKMSEISTDFTPFPHRKGTKLMFEYIIAWNQDEESKIGEFSEWLAKFYDYLEPFVSKEPRVGYVNHIDLDIGGIDWRNKSSTTNAVEIARNWGERYFSSNYERLVKAKTLIDPNNVFNHPQSIPPMMKFEEIYMLKEL

SEQ ID NO: 50 - DNA, 1563 bp, synthetic construct
  misc_feature 1..1563: note = CBS69-P93479-no signal peptide-strep Tag
  source 1..1563: mol_type = other DNA
  CDS 1..1563
atgggtgatgttaacgataacttgttgtcctcttgcttgaactctcatggtgttcataacttcactactttgtctaccgataccaactccgattacttcaagttgttgcatgcttctatgcaaaaccctttgtttgctaagccaaccgtttctaagccatcttttatcgttatgccaggctccaaagaagaattgtcatctactgttcattgctgcactagagaatcttggaccattagattgagatctggtggtcattcttacgagggtttgtcttatactgctgatactccattcgttatcgtcgacatgatgaacttgaacagaatctccatcgatgtcttgtctgaaactgcttgggttgaatctggtgctactttgggtgaactgtattatgctattgctcaatccactgataccttgggttttactgctggttggtgtccaactgttggttcaggtggtcatatttctggtggtggttttggtatgatgtctagaaagtatggtttggctgctgataacgttgttgatgccattttgattgattccaacggtgctatcttggacagagaaaaaatgggtgatgatgttttctgggctattagaggcggtggtggtggcgtttggggtgcaatctatgcttggaaaatcaagttattgccagtgccagaaaagttgaccgttttcagagttactaagaacgtcggtattgaagatgcctcttctttgttgcacaaatggcaatatgttgccgatgaattggatgaggatttcaccgtttctgttttaggtggtgttaatggtaatgatgcctggttgatgttcttgggtttacatttgggtagaaaggatgctgccaagaccattattgacgaaaagtttccagaattgggcctagtcgacaaagaatttcaagaaatgtcttggggtgaatcgatggcttttttgtctggtttggataccatctctgaactgaacaacagattcttgaagttcgacgaaagagctttcaagaccaaggttgatttcaccaaagtttccgttccattgaacgtttttagacacgctttggaaatgttgtctgaacaaccaggtggtttcattgctttgaatggtttcggtggtaagatgtccgaaatttctactgactttaccccatttccacacagaaaaggtacgaagttgatgtttgagtacattattgcctggaaccaggacgaagaatctaagattggtgaattttctgaatggctggccaagttctatgattacttggaaccattcgtttccaaagaacctagagttggttacgttaaccacatcgatttggatattggtggtatcgattggaggaacaaatcttctactaccaacgctgttgaaattgctagaaactggggtgaaagatacttcagctctaactacgaaagattggttaaggccaagactttgatcgatccaaacaacgttttcaaccatccacaatctattccaccaatgatgaagtttgaagagatctacatgctgaaagaattgtggtcacatccacaatttgaaaag

SEQ ID NO: 51 - protein, 521 aa, synthetic construct
  REGION 1..521: note = Synthetic Construct
MGDVNDNLLSSCLNSHGVHNFTTLSTDTNSDYFKLLHASMQNPLFAKPTVSKPSFIVMPGSKEELSSTVHCCTRESWTIRLRSGGHSYEGLSYTADTPFVIVDMMNLNRISIDVLSETAWVESGATLGELYYAIAQSTDTLGFTAGWCPTVGSGGHISGGGFGMMSRKYGLAADNVVDAILIDSNGAILDREKMGDDVFWAIRGGGGGVWGAIYAWKIKLLPVPEKLTVFRVTKNVGIEDASSLLHKWQYVADELDEDFTVSVLGGVNGNDAWLMFLGLHLGRKDAAKTIIDEKFPELGLVDKEFQEMSWGESMAFLSGLDTISELNNRFLKFDERAFKTKVDFTKVSVPLNVFRHALEMLSEQPGGFIALNGFGGKMSEISTDFTPFPHRKGTKLMFEYIIAWNQDEESKIGEFSEWLAKFYDYLEPFVSKEPRVGYVNHIDLDIGGIDWRNKSSTTNAVEIARNWGERYFSSNYERLVKAKTLIDPNNVFNHPQSIPPMMKFEEIYMLKELWSHPQFEK

SEQ ID NO: 52 - DNA, 1620 bp, synthetic construct
  misc_feature 1..1620: note = CBS69-P93479- truncated alpha factor -no signal peptide-strep Tag
  source 1..1620: mol_type = other DNA
  CDS 1..1620
atgagatttccttccatcttcacggctgtgctatttgcagcatcctccgcacttgcaatgggtgatgttaacgataacttgttgtcctcttgcttgaactctcatggtgttcataacttcactactttgtctaccgataccaactccgattacttcaagttgttgcatgcttctatgcaaaaccctttgtttgctaagccaaccgtttctaagccatcttttatcgttatgccaggctccaaagaagaattgtcatctactgttcattgctgcactagagaatcttggaccattagattgagatctggtggtcattcttacgagggtttgtcttatactgctgatactccattcgttatcgtcgacatgatgaacttgaacagaatctccatcgatgtcttgtctgaaactgcttgggttgaatctggtgctactttgggtgaactgtattatgctattgctcaatccactgataccttgggttttactgctggttggtgtccaactgttggttcaggtggtcatatttctggtggtggttttggtatgatgtctagaaagtatggtttggctgctgataacgttgttgatgccattttgattgattccaacggtgctatcttggacagagaaaaaatgggtgatgatgttttctgggctattagaggcggtggtggtggcgtttggggtgcaatctatgcttggaaaatcaagttattgccagtgccagaaaagttgaccgttttcagagttactaagaacgtcggtattgaagatgcctcttctttgttgcacaaatggcaatatgttgccgatgaattggatgaggatttcaccgtttctgttttaggtggtgttaatggtaatgatgcctggttgatgttcttgggtttacatttgggtagaaaggatgctgccaagaccattattgacgaaaagtttccagaattgggcctagtcgacaaagaatttcaagaaatgtcttggggtgaatcgatggcttttttgtctggtttggataccatctctgaactgaacaacagattcttgaagttcgacgaaagagctttcaagaccaaggttgatttcaccaaagtttccgttccattgaacgtttttagacacgctttggaaatgttgtctgaacaaccaggtggtttcattgctttgaatggtttcggtggtaagatgtccgaaatttctactgactttaccccatttccacacagaaaaggtacgaagttgatgtttgagtacattattgcctggaaccaggacgaagaatctaagattggtgaattttctgaatggctggccaagttctatgattacttggaaccattcgtttccaaagaacctagagttggttacgttaaccacatcgatttggatattggtggtatcgattggaggaacaaatcttctactaccaacgctgttgaaattgctagaaactggggtgaaagatacttcagctctaactacgaaagattggttaaggccaagactttgatcgatccaaacaacgttttcaaccatccacaatctattccaccaatgatgaagtttgaagagatctacatgctgaaagaattgtggtcacatccacaatttgaaaag

SEQ ID NO: 53 - protein, 540 aa, synthetic construct
  REGION 1..540: note = Synthetic Construct
MRFPSIFTAVLFAASSALAMGDVNDNLLSSCLNSHGVHNFTTLSTDTNSDYFKLLHASMQNPLFAKPTVSKPSFIVMPGSKEELSSTVHCCTRESWTIRLRSGGHSYEGLSYTADTPFVIVDMMNLNRISIDVLSETAWVESGATLGELYYAIAQSTDTLGFTAGWCPTVGSGGHISGGGFGMMSRKYGLAADNVVDAILIDSNGAILDREKMGDDVFWAIRGGGGGVWGAIYAWKIKLLPVPEKLTVFRVTKNVGIEDASSLLHKWQYVADELDEDFTVSVLGGVNGNDAWLMFLGLHLGRKDAAKTIIDEKFPELGLVDKEFQEMSWGESMAFLSGLDTISELNNRFLKFDERAFKTKVDFTKVSVPLNVFRHALEMLSEQPGGFIALNGFGGKMSEISTDFTPFPHRKGTKLMFEYIIAWNQDEESKIGEFSEWLAKFYDYLEPFVSKEPRVGYVNHIDLDIGGIDWRNKSSTTNAVEIARNWGERYFSSNYERLVKAKTLIDPNNVFNHPQSIPPMMKFEEIYMLKELWSHPQFEK

SEQ ID NO: 54 - DNA, 1830 bp, synthetic construct
  misc_feature 1..1830: note = CBS69-P93479-full alpha factor -no signal peptide-strep Tag
  source 1..1830: mol_type = other DNA
  CDS 1..1830
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatgggtgatgttaacgataacttgttgtcctcttgcttgaactctcatggtgttcataacttcactactttgtctaccgataccaactccgattacttcaagttgttgcatgcttctatgcaaaaccctttgtttgctaagccaaccgtttctaagccatcttttatcgttatgccaggctccaaagaagaattgtcatctactgttcattgctgcactagagaatcttggaccattagattgagatctggtggtcattcttacgagggtttgtcttatactgctgatactccattcgttatcgtcgacatgatgaacttgaacagaatctccatcgatgtcttgtctgaaactgcttgggttgaatctggtgctactttgggtgaactgtattatgctattgctcaatccactgataccttgggttttactgctggttggtgtccaactgttggttcaggtggtcatatttctggtggtggttttggtatgatgtctagaaagtatggtttggctgctgataacgttgttgatgccattttgattgattccaacggtgctatcttggacagagaaaaaatgggtgatgatgttttctgggctattagaggcggtggtggtggcgtttggggtgcaatctatgcttggaaaatcaagttattgccagtgccagaaaagttgaccgttttcagagttactaagaacgtcggtattgaagatgcctcttctttgttgcacaaatggcaatatgttgccgatgaattggatgaggatttcaccgtttctgttttaggtggtgttaatggtaatgatgcctggttgatgttcttgggtttacatttgggtagaaaggatgctgccaagaccattattgacgaaaagtttccagaattgggcctagtcgacaaagaatttcaagaaatgtcttggggtgaatcgatggcttttttgtctggtttggataccatctctgaactgaacaacagattcttgaagttcgacgaaagagctttcaagaccaaggttgatttcaccaaagtttccgttccattgaacgtttttagacacgctttggaaatgttgtctgaacaaccaggtggtttcattgctttgaatggtttcggtggtaagatgtccgaaatttctactgactttaccccatttccacacagaaaaggtacgaagttgatgtttgagtacattattgcctggaaccaggacgaagaatctaagattggtgaattttctgaatggctggccaagttctatgattacttggaaccattcgtttccaaagaacctagagttggttacgttaaccacatcgatttggatattggtggtatcgattggaggaacaaatcttctactaccaacgctgttgaaattgctagaaactggggtgaaagatacttcagctctaactacgaaagattggttaaggccaagactttgatcgatccaaacaacgttttcaaccatccacaatctattccaccaatgatgaagtttgaagagatctacatgctgaaagaattgtggtcacatccacaatttgaaaag

SEQ ID NO: 55 - protein, 610 aa, synthetic construct
  REGION 1..610: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAMGDVNDNLLSSCLNSHGVHNFTTLSTDTNSDYFKLLHASMQNPLFAKPTVSKPSFIVMPGSKEELSSTVHCCTRESWTIRLRSGGHSYEGLSYTADTPFVIVDMMNLNRISIDVLSETAWVESGATLGELYYAIAQSTDTLGFTAGWCPTVGSGGHISGGGFGMMSRKYGLAADNVVDAILIDSNGAILDREKMGDDVFWAIRGGGGGVWGAIYAWKIKLLPVPEKLTVFRVTKNVGIEDASSLLHKWQYVADELDEDFTVSVLGGVNGNDAWLMFLGLHLGRKDAAKTIIDEKFPELGLVDKEFQEMSWGESMAFLSGLDTISELNNRFLKFDERAFKTKVDFTKVSVPLNVFRHALEMLSEQPGGFIALNGFGGKMSEISTDFTPFPHRKGTKLMFEYIIAWNQDEESKIGEFSEWLAKFYDYLEPFVSKEPRVGYVNHIDLDIGGIDWRNKSSTTNAVEIARNWGERYFSSNYERLVKAKTLIDPNNVFNHPQSIPPMMKFEEIYMLKELWSHPQFEK

SEQ ID NO: 56 - DNA, 1611 bp, synthetic construct
  misc_feature 1..1611: note = CBS86-A0A1U8G0D2
  source 1..1611: mol_type = other DNA
  CDS 1..1611
atgatgggttccttgcaaatcttgttcgtcttgttgttctccctggttttgattaagtgctactctcaagaggatttcttggagtgtttgtccaagtactctaagtccaacgttgaacaaaacgtttacactccaacttctccaacctactcctctattttggaatacgctcaaaagaacccaagatgggttaattcttctcacccaaactttattgcctcgccaagaaaagaatctgacatcaagccaattatcttgtgcgctaagaaattgggcttgcagattaagattaagtccggtggtcatgattacgagggtatttcttacagatctaagtccccattcgttatgttggacttgtctaacttgaacaagatcgagatcaactggaagaaagaaaccgtttgggttcaaactggtgctactttgggtcaactgtattatgctattgccaacaagtctaacgttcatggttttccaggtggtgtttgtttctctattggtactggtggtattatttctggtggtggtataggtactatgatgagaaaatttggtgttgccgctgataacgttgttgatgctagagttatggatgtcaacggtaagatcttggacaagaaaaagatgaaggaagatttgttctgggccattagaggtggtggtggcgcttcttttggtgttattttggcttggaaattgaagttggttagggttccagaaaaggtcactgtttttaacgtctacaaaaagttggaaggcaaccagaacttgttgcaaaagtgggaaaacattgctcatcaattgaccgacaagttgttgatcagagtcatcattcaaaacgacggtactggtaaggataagtacatcgaagttatcttccaagccttgttcttgggtttagttgatgaattgatccccttgctgaaagaaaagttcccagaattcgatttggaaaagaaggactgtttccaagaaccagttgttgactgttctaacagaccatgcattaagaaagaatgcagagaaactacctggatcggttctgttttgtttttctacggtagaaggaccaacgaatccttggaagttttgttggataagtccattccaacgcagaagaactacaacaaaggtacttctgatttcgtcaagaccccaattccagaatctggttggaaaatggttcaaaggttgttcttagaagaggaacgtccacaaatgatcttggaaccattaggtggtaagttggacgaaatctccaaatccgaaatttcattcccacacagaaagggcaacttgtacaacattcaatacttggttaactggggtgacaactccgaaaatgtttcctctcaaaaattggcctggatgcgtaagttgtacaaagatatggaaccatacgttgccaactctccaagaactgcttatttgaactacagagatttggacttcggttccaatgatgaaggttactcttactccaaagctaagatttggggtgaaaagtacttcaacggcaactttaagagattggccaaggttaagtctaaggtcgatccaaacaatttcttcagaaacgaacaatcgatcccaccatatcaatcccat

SEQ ID NO: 57 - protein, 537 aa, synthetic construct
  REGION 1..537: note = Synthetic Construct
MMGSLQILFVLLFSLVLIKCYSQEDFLECLSKYSKSNVEQNVYTPTSPTYSSILEYAQKNPRWVNSSHPNFIASPRKESDIKPIILCAKKLGLQIKIKSGGHDYEGISYRSKSPFVMLDLSNLNKIEINWKKETVWVQTGATLGQLYYAIANKSNVHGFPGGVCFSIGTGGIISGGGIGTMMRKFGVAADNVVDARVMDVNGKILDKKKMKEDLFWAIRGGGGASFGVILAWKLKLVRVPEKVTVFNVYKKLEGNQNLLQKWENIAHQLTDKLLIRVIIQNDGTGKDKYIEVIFQALFLGLVDELIPLLKEKFPEFDLEKKDCFQEPVVDCSNRPCIKKECRETTWIGSVLFFYGRRTNESLEVLLDKSIPTQKNYNKGTSDFVKTPIPESGWKMVQRLFLEEERPQMILEPLGGKLDEISKSEISFPHRKGNLYNIQYLVNWGDNSENVSSQKLAWMRKLYKDMEPYVANSPRTAYLNYRDLDFGSNDEGYSYSKAKIWGEKYFNGNFKRLAKVKSKVDPNNFFRNEQSIPPYQSH

SEQ ID NO: 58 - DNA, 1551 bp, synthetic construct
  misc_feature 1..1551: note = CBS86-A0A1U8G0D2-no signal peptide
  source 1..1551: mol_type = other DNA
  CDS 1..1551
atgtctcaagaggatttcttggagtgtttgtccaagtactctaagtccaacgttgaacaaaacgtttacactccaacttctccaacctactcctctattttggaatacgctcaaaagaacccaagatgggttaattcttctcacccaaactttattgcctcgccaagaaaagaatctgacatcaagccaattatcttgtgcgctaagaaattgggcttgcagattaagattaagtccggtggtcatgattacgagggtatttcttacagatctaagtccccattcgttatgttggacttgtctaacttgaacaagatcgagatcaactggaagaaagaaaccgtttgggttcaaactggtgctactttgggtcaactgtattatgctattgccaacaagtctaacgttcatggttttccaggtggtgtttgtttctctattggtactggtggtattatttctggtggtggtataggtactatgatgagaaaatttggtgttgccgctgataacgttgttgatgctagagttatggatgtcaacggtaagatcttggacaagaaaaagatgaaggaagatttgttctgggccattagaggtggtggtggcgcttcttttggtgttattttggcttggaaattgaagttggttagggttccagaaaaggtcactgtttttaacgtctacaaaaagttggaaggcaaccagaacttgttgcaaaagtgggaaaacattgctcatcaattgaccgacaagttgttgatcagagtcatcattcaaaacgacggtactggtaaggataagtacatcgaagttatcttccaagccttgttcttgggtttagttgatgaattgatccccttgctgaaagaaaagttcccagaattcgatttggaaaagaaggactgtttccaagaaccagttgttgactgttctaacagaccatgcattaagaaagaatgcagagaaactacctggatcggttctgttttgtttttctacggtagaaggaccaacgaatccttggaagttttgttggataagtccattccaacgcagaagaactacaacaaaggtacttctgatttcgtcaagaccccaattccagaatctggttggaaaatggttcaaaggttgttcttagaagaggaacgtccacaaatgatcttggaaccattaggtggtaagttggacgaaatctccaaatccgaaatttcattcccacacagaaagggcaacttgtacaacattcaatacttggttaactggggtgacaactccgaaaatgtttcctctcaaaaattggcctggatgcgtaagttgtacaaagatatggaaccatacgttgccaactctccaagaactgcttatttgaactacagagatttggacttcggttccaatgatgaaggttactcttactccaaagctaagatttggggtgaaaagtacttcaacggcaactttaagagattggccaaggttaagtctaaggtcgatccaaacaatttcttcagaaacgaacaatcgatcccaccatatcaatcccat

SEQ ID NO: 59 - protein, 517 aa, synthetic construct
  REGION 1..517: note = Synthetic Construct
MSQEDFLECLSKYSKSNVEQNVYTPTSPTYSSILEYAQKNPRWVNSSHPNFIASPRKESDIKPIILCAKKLGLQIKIKSGGHDYEGISYRSKSPFVMLDLSNLNKIEINWKKETVWVQTGATLGQLYYAIANKSNVHGFPGGVCFSIGTGGIISGGGIGTMMRKFGVAADNVVDARVMDVNGKILDKKKMKEDLFWAIRGGGGASFGVILAWKLKLVRVPEKVTVFNVYKKLEGNQNLLQKWENIAHQLTDKLLIRVIIQNDGTGKDKYIEVIFQALFLGLVDELIPLLKEKFPEFDLEKKDCFQEPVVDCSNRPCIKKECRETTWIGSVLFFYGRRTNESLEVLLDKSIPTQKNYNKGTSDFVKTPIPESGWKMVQRLFLEEERPQMILEPLGGKLDEISKSEISFPHRKGNLYNIQYLVNWGDNSENVSSQKLAWMRKLYKDMEPYVANSPRTAYLNYRDLDFGSNDEGYSYSKAKIWGEKYFNGNFKRLAKVKSKVDPNNFFRNEQSIPPYQSH

SEQ ID NO: 60 - DNA, 1575 bp, synthetic construct
  misc_feature 1..1575: note = CBS86-A0A1U8G0D2-no signal peptide-strep Tag
  source 1..1575: mol_type = other DNA
  CDS 1..1575
atgtctcaagaggatttcttggagtgtttgtccaagtactctaagtccaacgttgaacaaaacgtttacactccaacttctccaacctactcctctattttggaatacgctcaaaagaacccaagatgggttaattcttctcacccaaactttattgcctcgccaagaaaagaatctgacatcaagccaattatcttgtgcgctaagaaattgggcttgcagattaagattaagtccggtggtcatgattacgagggtatttcttacagatctaagtccccattcgttatgttggacttgtctaacttgaacaagatcgagatcaactggaagaaagaaaccgtttgggttcaaactggtgctactttgggtcaactgtattatgctattgccaacaagtctaacgttcatggttttccaggtggtgtttgtttctctattggtactggtggtattatttctggtggtggtataggtactatgatgagaaaatttggtgttgccgctgataacgttgttgatgctagagttatggatgtcaacggtaagatcttggacaagaaaaagatgaaggaagatttgttctgggccattagaggtggtggtggcgcttcttttggtgttattttggcttggaaattgaagttggttagggttccagaaaaggtcactgtttttaacgtctacaaaaagttggaaggcaaccagaacttgttgcaaaagtgggaaaacattgctcatcaattgaccgacaagttgttgatcagagtcatcattcaaaacgacggtactggtaaggataagtacatcgaagttatcttccaagccttgttcttgggtttagttgatgaattgatccccttgctgaaagaaaagttcccagaattcgatttggaaaagaaggactgtttccaagaaccagttgttgactgttctaacagaccatgcattaagaaagaatgcagagaaactacctggatcggttctgttttgtttttctacggtagaaggaccaacgaatccttggaagttttgttggataagtccattccaacgcagaagaactacaacaaaggtacttctgatttcgtcaagaccccaattccagaatctggttggaaaatggttcaaaggttgttcttagaagaggaacgtccacaaatgatcttggaaccattaggtggtaagttggacgaaatctccaaatccgaaatttcattcccacacagaaagggcaacttgtacaacattcaatacttggttaactggggtgacaactccgaaaatgtttcctctcaaaaattggcctggatgcgtaagttgtacaaagatatggaaccatacgttgccaactctccaagaactgcttatttgaactacagagatttggacttcggttccaatgatgaaggttactcttactccaaagctaagatttggggtgaaaagtacttcaacggcaactttaagagattggccaaggttaagtctaaggtcgatccaaacaatttcttcagaaacgaacaatcgatcccaccatatcaatcccattggtcacatccacaatttgaaaag

SEQ ID NO: 61 - protein, 525 aa, synthetic construct
  REGION 1..525: note = Synthetic Construct
MSQEDFLECLSKYSKSNVEQNVYTPTSPTYSSILEYAQKNPRWVNSSHPNFIASPRKESDIKPIILCAKKLGLQIKIKSGGHDYEGISYRSKSPFVMLDLSNLNKIEINWKKETVWVQTGATLGQLYYAIANKSNVHGFPGGVCFSIGTGGIISGGGIGTMMRKFGVAADNVVDARVMDVNGKILDKKKMKEDLFWAIRGGGGASFGVILAWKLKLVRVPEKVTVFNVYKKLEGNQNLLQKWENIAHQLTDKLLIRVIIQNDGTGKDKYIEVIFQALFLGLVDELIPLLKEKFPEFDLEKKDCFQEPVVDCSNRPCIKKECRETTWIGSVLFFYGRRTNESLEVLLDKSIPTQKNYNKGTSDFVKTPIPESGWKMVQRLFLEEERPQMILEPLGGKLDEISKSEISFPHRKGNLYNIQYLVNWGDNSENVSSQKLAWMRKLYKDMEPYVANSPRTAYLNYRDLDFGSNDEGYSYSKAKIWGEKYFNGNFKRLAKVKSKVDPNNFFRNEQSIPPYQSHWSHPQFEK

SEQ ID NO: 62 - DNA, 1632 bp, synthetic construct
  misc_feature 1..1632: note = CBS86-A0A1U8G0D2-truncated alpha factor -no signal peptide-strep Tag
  source 1..1632: mol_type = other DNA
  CDS 1..1632
atgagatttccttccatcttcacggctgtgctatttgcagcatcctccgcacttgcaatgtctcaagaggatttcttggagtgtttgtccaagtactctaagtccaacgttgaacaaaacgtttacactccaacttctccaacctactcctctattttggaatacgctcaaaagaacccaagatgggttaattcttctcacccaaactttattgcctcgccaagaaaagaatctgacatcaagccaattatcttgtgcgctaagaaattgggcttgcagattaagattaagtccggtggtcatgattacgagggtatttcttacagatctaagtccccattcgttatgttggacttgtctaacttgaacaagatcgagatcaactggaagaaagaaaccgtttgggttcaaactggtgctactttgggtcaactgtattatgctattgccaacaagtctaacgttcatggttttccaggtggtgtttgtttctctattggtactggtggtattatttctggtggtggtataggtactatgatgagaaaatttggtgttgccgctgataacgttgttgatgctagagttatggatgtcaacggtaagatcttggacaagaaaaagatgaaggaagatttgttctgggccattagaggtggtggtggcgcttcttttggtgttattttggcttggaaattgaagttggttagggttccagaaaaggtcactgtttttaacgtctacaaaaagttggaaggcaaccagaacttgttgcaaaagtgggaaaacattgctcatcaattgaccgacaagttgttgatcagagtcatcattcaaaacgacggtactggtaaggataagtacatcgaagttatcttccaagccttgttcttgggtttagttgatgaattgatccccttgctgaaagaaaagttcccagaattcgatttggaaaagaaggactgtttccaagaaccagttgttgactgttctaacagaccatgcattaagaaagaatgcagagaaactacctggatcggttctgttttgtttttctacggtagaaggaccaacgaatccttggaagttttgttggataagtccattccaacgcagaagaactacaacaaaggtacttctgatttcgtcaagaccccaattccagaatctggttggaaaatggttcaaaggttgttcttagaagaggaacgtccacaaatgatcttggaaccattaggtggtaagttggacgaaatctccaaatccgaaatttcattcccacacagaaagggcaacttgtacaacattcaatacttggttaactggggtgacaactccgaaaatgtttcctctcaaaaattggcctggatgcgtaagttgtacaaagatatggaaccatacgttgccaactctccaagaactgcttatttgaactacagagatttggacttcggttccaatgatgaaggttactcttactccaaagctaagatttggggtgaaaagtacttcaacggcaactttaagagattggccaaggttaagtctaaggtcgatccaaacaatttcttcagaaacgaacaatcgatcccaccatatcaatcccattggtcacatccacaatttgaaaag

SEQ ID NO: 63 - protein, 544 aa, synthetic construct
  REGION 1..544: note = Synthetic Construct
MRFPSIFTAVLFAASSALAMSQEDFLECLSKYSKSNVEQNVYTPTSPTYSSILEYAQKNPRWVNSSHPNFIASPRKESDIKPIILCAKKLGLQIKIKSGGHDYEGISYRSKSPFVMLDLSNLNKIEINWKKETVWVQTGATLGQLYYAIANKSNVHGFPGGVCFSIGTGGIISGGGIGTMMRKFGVAADNVVDARVMDVNGKILDKKKMKEDLFWAIRGGGGASFGVILAWKLKLVRVPEKVTVFNVYKKLEGNQNLLQKWENIAHQLTDKLLIRVIIQNDGTGKDKYIEVIFQALFLGLVDELIPLLKEKFPEFDLEKKDCFQEPVVDCSNRPCIKKECRETTWIGSVLFFYGRRTNESLEVLLDKSIPTQKNYNKGTSDFVKTPIPESGWKMVQRLFLEEERPQMILEPLGGKLDEISKSEISFPHRKGNLYNIQYLVNWGDNSENVSSQKLAWMRKLYKDMEPYVANSPRTAYLNYRDLDFGSNDEGYSYSKAKIWGEKYFNGNFKRLAKVKSKVDPNNFFRNEQSIPPYQSHWSHPQFEK

SEQ ID NO: 64 - DNA, 1842 bp, synthetic construct
  misc_feature 1..1842: note = CBS86-A0A1U8G0D2-full alpha factor -no signal peptide-strep Tag
  source 1..1842: mol_type = other DNA
  CDS 1..1842
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatgtctcaagaggatttcttggagtgtttgtccaagtactctaagtccaacgttgaacaaaacgtttacactccaacttctccaacctactcctctattttggaatacgctcaaaagaacccaagatgggttaattcttctcacccaaactttattgcctcgccaagaaaagaatctgacatcaagccaattatcttgtgcgctaagaaattgggcttgcagattaagattaagtccggtggtcatgattacgagggtatttcttacagatctaagtccccattcgttatgttggacttgtctaacttgaacaagatcgagatcaactggaagaaagaaaccgtttgggttcaaactggtgctactttgggtcaactgtattatgctattgccaacaagtctaacgttcatggttttccaggtggtgtttgtttctctattggtactggtggtattatttctggtggtggtataggtactatgatgagaaaatttggtgttgccgctgataacgttgttgatgctagagttatggatgtcaacggtaagatcttggacaagaaaaagatgaaggaagatttgttctgggccattagaggtggtggtggcgcttcttttggtgttattttggcttggaaattgaagttggttagggttccagaaaaggtcactgtttttaacgtctacaaaaagttggaaggcaaccagaacttgttgcaaaagtgggaaaacattgctcatcaattgaccgacaagttgttgatcagagtcatcattcaaaacgacggtactggtaaggataagtacatcgaagttatcttccaagccttgttcttgggtttagttgatgaattgatccccttgctgaaagaaaagttcccagaattcgatttggaaaagaaggactgtttccaagaaccagttgttgactgttctaacagaccatgcattaagaaagaatgcagagaaactacctggatcggttctgttttgtttttctacggtagaaggaccaacgaatccttggaagttttgttggataagtccattccaacgcagaagaactacaacaaaggtacttctgatttcgtcaagaccccaattccagaatctggttggaaaatggttcaaaggttgttcttagaagaggaacgtccacaaatgatcttggaaccattaggtggtaagttggacgaaatctccaaatccgaaatttcattcccacacagaaagggcaacttgtacaacattcaatacttggttaactggggtgacaactccgaaaatgtttcctctcaaaaattggcctggatgcgtaagttgtacaaagatatggaaccatacgttgccaactctccaagaactgcttatttgaactacagagatttggacttcggttccaatgatgaaggttactcttactccaaagctaagatttggggtgaaaagtacttcaacggcaactttaagagattggccaaggttaagtctaaggtcgatccaaacaatttcttcagaaacgaacaatcgatcccaccatatcaatcccattggtcacatccacaatttgaaaag

SEQ ID NO: 65 - protein, 614 aa, synthetic construct
  REGION 1..614: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAMSQEDFLECLSKYSKSNVEQNVYTPTSPTYSSILEYAQKNPRWVNSSHPNFIASPRKESDIKPIILCAKKLGLQIKIKSGGHDYEGISYRSKSPFVMLDLSNLNKIEINWKKETVWVQTGATLGQLYYAIANKSNVHGFPGGVCFSIGTGGIISGGGIGTMMRKFGVAADNVVDARVMDVNGKILDKKKMKEDLFWAIRGGGGASFGVILAWKLKLVRVPEKVTVFNVYKKLEGNQNLLQKWENIAHQLTDKLLIRVIIQNDGTGKDKYIEVIFQALFLGLVDELIPLLKEKFPEFDLEKKDCFQEPVVDCSNRPCIKKECRETTWIGSVLFFYGRRTNESLEVLLDKSIPTQKNYNKGTSDFVKTPIPESGWKMVQRLFLEEERPQMILEPLGGKLDEISKSEISFPHRKGNLYNIQYLVNWGDNSENVSSQKLAWMRKLYKDMEPYVANSPRTAYLNYRDLDFGSNDEGYSYSKAKIWGEKYFNGNFKRLAKVKSKVDPNNFFRNEQSIPPYQSHWSHPQFEK

SEQ ID NO: 66 - DNA, 1629 bp, synthetic construct
  misc_feature 1..1629: note = CBS94-A0A1S3XA06
  source 1..1629: mol_type = other DNA
  CDS 1..1629
atgggtagattgcagttgttctcttgcttgttggtttccttcttgttgttgattgaagctaacgcccaacaagagttcttgcaatgtatctactctcactcctccgagaatattaagaagcacattcatttcccaaacagcccaacttactcttcgttgttggaatacgctcaaaagaatccaagatggctgaattcttctttcgcccatccaattttcattatcgctccaaagaacgaatccgaaatccagccaattatcttgtgctctcaaaagttcggcttgcaaatcagagttaagtctggtggtcatgattacgaaggtttgtctttcagatctgtcactaacacccaattcgttatcttggacttgatcaacttggacgatatcagaatcgacaagaacgaagaaactgcttggattcaaactggtgttactatgggtcagttgtactacgaaattgccaagaagtctaagatcttggcttttccaggtggtctatatccatctgttggttctggtggtttgatttcaggtggtggtattggtactttgatgagaaagtttggtttgtccgctgataacgtcattgatgctagagttatggatgtcaacggtagaatcttggacaaaaagtctatgggcgaagatatgttctgggctattagaggtggtggcggttcttcttttggtgttattttggcttggaagatcaagttggttagaatcccaccaaaggttactgttttcaccgttagaagaaagttggaaggtaacaccatcaacttgttgcaaagatggcaaaacgtttcccataagttcccaaaggatttgttcatgagagtcttgatccaaaacatgggtttcggtaacgaaaagatcgttcaagtttcattccagggtttgtttttgggtagagcttctgaattgacccctttgttgaatcaaactttcccagaattcggtttggacccaaaaaactgtttccaagatccagttgttaactgcaacgaattgcccttcattaagaaagaatgcttcgaagttccatggatcagatctaccttgtactttgcttctaagaggaccaacaactccattgaattcttggttaacaacaccgtttctcagaccaagaactaccaaaaagctacttccgatttcgttaagaccccaatgcaagaagaggtttggggtatgatcaaagacttgttcttggaagaagagaagcccatgattattttggaccctttaggtggtaagttggacgaaatttctgaatccgaattgccatttccacacagaaagggtaacttgttcaacatccagtacatggttaattggggtgacaactccgaatctatctcctccaaaaaaatcaaatggctgcgtgagttgtacgagaagatgaagccatttttgtctcattgtcctagaaccgcttacttgaactacagagatttggacttcggtactaatgatggtgtctacagatactctaaagccaaagtttggggtgctaagtacttcaacgaaaactttgaaagattggccaaggtcaagtctaaagttgatccaggtaatttcttcaggtacgaacaatctattccaccattctctgtttccacc

SEQ ID NO: 67 - protein, 543 aa, synthetic construct
  REGION 1..543: note = Synthetic Construct
MGRLQLFSCLLVSFLLLIEANAQQEFLQCIYSHSSENIKKHIHFPNSPTYSSLLEYAQKNPRWLNSSFAHPIFIIAPKNESEIQPIILCSQKFGLQIRVKSGGHDYEGLSFRSVTNTQFVILDLINLDDIRIDKNEETAWIQTGVTMGQLYYEIAKKSKILAFPGGLYPSVGSGGLISGGGIGTLMRKFGLSADNVIDARVMDVNGRILDKKSMGEDMFWAIRGGGGSSFGVILAWKIKLVRIPPKVTVFTVRRKLEGNTINLLQRWQNVSHKFPKDLFMRVLIQNMGFGNEKIVQVSFQGLFLGRASELTPLLNQTFPEFGLDPKNCFQDPVVNCNELPFIKKECFEVPWIRSTLYFASKRTNNSIEFLVNNTVSQTKNYQKATSDFVKTPMQEEVWGMIKDLFLEEEKPMIILDPLGGKLDEISESELPFPHRKGNLFNIQYMVNWGDNSESISSKKIKWLRELYEKMKPFLSHCPRTAYLNYRDLDFGTNDGVYRYSKAKVWGAKYFNENFERLAKVKSKVDPGNFFRYEQSIPPFSVST

SEQ ID NO: 68 - DNA, 1569 bp, synthetic construct
  misc_feature 1..1569: note = CBS94-A0A1S3XA06-no signal peptide
  source 1..1569: mol_type = other DNA
  CDS 1..1569
atggcccaacaagagttcttgcaatgtatctactctcactcctccgagaatattaagaagcacattcatttcccaaacagcccaacttactcttcgttgttggaatacgctcaaaagaatccaagatggctgaattcttctttcgcccatccaattttcattatcgctccaaagaacgaatccgaaatccagccaattatcttgtgctctcaaaagttcggcttgcaaatcagagttaagtctggtggtcatgattacgaaggtttgtctttcagatctgtcactaacacccaattcgttatcttggacttgatcaacttggacgatatcagaatcgacaagaacgaagaaactgcttggattcaaactggtgttactatgggtcagttgtactacgaaattgccaagaagtctaagatcttggcttttccaggtggtctatatccatctgttggttctggtggtttgatttcaggtggtggtattggtactttgatgagaaagtttggtttgtccgctgataacgtcattgatgctagagttatggatgtcaacggtagaatcttggacaaaaagtctatgggcgaagatatgttctgggctattagaggtggtggcggttcttcttttggtgttattttggcttggaagatcaagttggttagaatcccaccaaaggttactgttttcaccgttagaagaaagttggaaggtaacaccatcaacttgttgcaaagatggcaaaacgtttcccataagttcccaaaggatttgttcatgagagtcttgatccaaaacatgggtttcggtaacgaaaagatcgttcaagtttcattccagggtttgtttttgggtagagcttctgaattgacccctttgttgaatcaaactttcccagaattcggtttggacccaaaaaactgtttccaagatccagttgttaactgcaacgaattgcccttcattaagaaagaatgcttcgaagttccatggatcagatctaccttgtactttgcttctaagaggaccaacaactccattgaattcttggttaacaacaccgtttctcagaccaagaactaccaaaaagctacttccgatttcgttaagaccccaatgcaagaagaggtttggggtatgatcaaagacttgttcttggaagaagagaagcccatgattattttggaccctttaggtggtaagttggacgaaatttctgaatccgaattgccatttccacacagaaagggtaacttgttcaacatccagtacatggttaattggggtgacaactccgaatctatctcctccaaaaaaatcaaatggctgcgtgagttgtacgagaagatgaagccatttttgtctcattgtcctagaaccgcttacttgaactacagagatttggacttcggtactaatgatggtgtctacagatactctaaagccaaagtttggggtgctaagtacttcaacgaaaactttgaaagattggccaaggtcaagtctaaagttgatccaggtaatttcttcaggtacgaacaatctattccaccattctctgtttccacc

SEQ ID NO: 69 - protein, 523 aa, synthetic construct
  REGION 1..523: note = Synthetic Construct
MAQQEFLQCIYSHSSENIKKHIHFPNSPTYSSLLEYAQKNPRWLNSSFAHPIFIIAPKNESEIQPIILCSQKFGLQIRVKSGGHDYEGLSFRSVTNTQFVILDLINLDDIRIDKNEETAWIQTGVTMGQLYYEIAKKSKILAFPGGLYPSVGSGGLISGGGIGTLMRKFGLSADNVIDARVMDVNGRILDKKSMGEDMFWAIRGGGGSSFGVILAWKIKLVRIPPKVTVFTVRRKLEGNTINLLQRWQNVSHKFPKDLFMRVLIQNMGFGNEKIVQVSFQGLFLGRASELTPLLNQTFPEFGLDPKNCFQDPVVNCNELPFIKKECFEVPWIRSTLYFASKRTNNSIEFLVNNTVSQTKNYQKATSDFVKTPMQEEVWGMIKDLFLEEEKPMIILDPLGGKLDEISESELPFPHRKGNLFNIQYMVNWGDNSESISSKKIKWLRELYEKMKPFLSHCPRTAYLNYRDLDFGTNDGVYRYSKAKVWGAKYFNENFERLAKVKSKVDPGNFFRYEQSIPPFSVST

SEQ ID NO: 70 - DNA, 1593 bp, synthetic construct
  misc_feature 1..1593: note = CBS94-A0A1S3XA06-no signal peptide-strep Tag
  source 1..1593: mol_type = other DNA
  CDS 1..1593
atggcccaacaagagttcttgcaatgtatctactctcactcctccgagaatattaagaagcacattcatttcccaaacagcccaacttactcttcgttgttggaatacgctcaaaagaatccaagatggctgaattcttctttcgcccatccaattttcattatcgctccaaagaacgaatccgaaatccagccaattatcttgtgctctcaaaagttcggcttgcaaatcagagttaagtctggtggtcatgattacgaaggtttgtctttcagatctgtcactaacacccaattcgttatcttggacttgatcaacttggacgatatcagaatcgacaagaacgaagaaactgcttggattcaaactggtgttactatgggtcagttgtactacgaaattgccaagaagtctaagatcttggcttttccaggtggtctatatccatctgttggttctggtggtttgatttcaggtggtggtattggtactttgatgagaaagtttggtttgtccgctgataacgtcattgatgctagagttatggatgtcaacggtagaatcttggacaaaaagtctatgggcgaagatatgttctgggctattagaggtggtggcggttcttcttttggtgttattttggcttggaagatcaagttggttagaatcccaccaaaggttactgttttcaccgttagaagaaagttggaaggtaacaccatcaacttgttgcaaagatggcaaaacgtttcccataagttcccaaaggatttgttcatgagagtcttgatccaaaacatgggtttcggtaacgaaaagatcgttcaagtttcattccagggtttgtttttgggtagagcttctgaattgacccctttgttgaatcaaactttcccagaattcggtttggacccaaaaaactgtttccaagatccagttgttaactgcaacgaattgcccttcattaagaaagaatgcttcgaagttccatggatcagatctaccttgtactttgcttctaagaggaccaacaactccattgaattcttggttaacaacaccgtttctcagaccaagaactaccaaaaagctacttccgatttcgttaagaccccaatgcaagaagaggtttggggtatgatcaaagacttgttcttggaagaagagaagcccatgattattttggaccctttaggtggtaagttggacgaaatttctgaatccgaattgccatttccacacagaaagggtaacttgttcaacatccagtacatggttaattggggtgacaactccgaatctatctcctccaaaaaaatcaaatggctgcgtgagttgtacgagaagatgaagccatttttgtctcattgtcctagaaccgcttacttgaactacagagatttggacttcggtactaatgatggtgtctacagatactctaaagccaaagtttggggtgctaagtacttcaacgaaaactttgaaagattggccaaggtcaagtctaaagttgatccaggtaatttcttcaggtacgaacaatctattccaccattctctgtttccacctggtcacatccacaatttgaaaag

SEQ ID NO: 71 - protein, 531 aa, synthetic construct
  REGION 1..531: note = Synthetic Construct
MAQQEFLQCIYSHSSENIKKHIHFPNSPTYSSLLEYAQKNPRWLNSSFAHPIFIIAPKNESEIQPIILCSQKFGLQIRVKSGGHDYEGLSFRSVTNTQFVILDLINLDDIRIDKNEETAWIQTGVTMGQLYYEIAKKSKILAFPGGLYPSVGSGGLISGGGIGTLMRKFGLSADNVIDARVMDVNGRILDKKSMGEDMFWAIRGGGGSSFGVILAWKIKLVRIPPKVTVFTVRRKLEGNTINLLQRWQNVSHKFPKDLFMRVLIQNMGFGNEKIVQVSFQGLFLGRASELTPLLNQTFPEFGLDPKNCFQDPVVNCNELPFIKKECFEVPWIRSTLYFASKRTNNSIEFLVNNTVSQTKNYQKATSDFVKTPMQEEVWGMIKDLFLEEEKPMIILDPLGGKLDEISESELPFPHRKGNLFNIQYMVNWGDNSESISSKKIKWLRELYEKMKPFLSHCPRTAYLNYRDLDFGTNDGVYRYSKAKVWGAKYFNENFERLAKVKSKVDPGNFFRYEQSIPPFSVSTWSHPQFEK

SEQ ID NO: 72 - DNA, 1650 bp, synthetic construct
  misc_feature 1..1650: note = CBS94-A0A1S3XA06-truncated alpha factor-no signal peptide-strep Tag
  source 1..1650: mol_type = other DNA
  CDS 1..1650
atgagatttccttccatcttcacggctgtgctatttgcagcatcctccgcacttgcaatggcccaacaagagttcttgcaatgtatctactctcactcctccgagaatattaagaagcacattcatttcccaaacagcccaacttactcttcgttgttggaatacgctcaaaagaatccaagatggctgaattcttctttcgcccatccaattttcattatcgctccaaagaacgaatccgaaatccagccaattatcttgtgctctcaaaagttcggcttgcaaatcagagttaagtctggtggtcatgattacgaaggtttgtctttcagatctgtcactaacacccaattcgttatcttggacttgatcaacttggacgatatcagaatcgacaagaacgaagaaactgcttggattcaaactggtgttactatgggtcagttgtactacgaaattgccaagaagtctaagatcttggcttttccaggtggtctatatccatctgttggttctggtggtttgatttcaggtggtggtattggtactttgatgagaaagtttggtttgtccgctgataacgtcattgatgctagagttatggatgtcaacggtagaatcttggacaaaaagtctatgggcgaagatatgttctgggctattagaggtggtggcggttcttcttttggtgttattttggcttggaagatcaagttggttagaatcccaccaaaggttactgttttcaccgttagaagaaagttggaaggtaacaccatcaacttgttgcaaagatggcaaaacgtttcccataagttcccaaaggatttgttcatgagagtcttgatccaaaacatgggtttcggtaacgaaaagatcgttcaagtttcattccagggtttgtttttgggtagagcttctgaattgacccctttgttgaatcaaactttcccagaattcggtttggacccaaaaaactgtttccaagatccagttgttaactgcaacgaattgcccttcattaagaaagaatgcttcgaagttccatggatcagatctaccttgtactttgcttctaagaggaccaacaactccattgaattcttggttaacaacaccgtttctcagaccaagaactaccaaaaagctacttccgatttcgttaagaccccaatgcaagaagaggtttggggtatgatcaaagacttgttcttggaagaagagaagcccatgattattttggaccctttaggtggtaagttggacgaaatttctgaatccgaattgccatttccacacagaaagggtaacttgttcaacatccagtacatggttaattggggtgacaactccgaatctatctcctccaaaaaaatcaaatggctgcgtgagttgtacgagaagatgaagccatttttgtctcattgtcctagaaccgcttacttgaactacagagatttggacttcggtactaatgatggtgtctacagatactctaaagccaaagtttggggtgctaagtacttcaacgaaaactttgaaagattggccaaggtcaagtctaaagttgatccaggtaatttcttcaggtacgaacaatctattccaccattctctgtttccacctggtcacatccacaatttgaaaag

SEQ ID NO: 73 - protein, 550 aa, synthetic construct
  REGION 1..550: note = Synthetic Construct
MRFPSIFTAVLFAASSALAMAQQEFLQCIYSHSSENIKKHIHFPNSPTYSSLLEYAQKNPRWLNSSFAHPIFIIAPKNESEIQPIILCSQKFGLQIRVKSGGHDYEGLSFRSVTNTQFVILDLINLDDIRIDKNEETAWIQTGVTMGQLYYEIAKKSKILAFPGGLYPSVGSGGLISGGGIGTLMRKFGLSADNVIDARVMDVNGRILDKKSMGEDMFWAIRGGGGSSFGVILAWKIKLVRIPPKVTVFTVRRKLEGNTINLLQRWQNVSHKFPKDLFMRVLIQNMGFGNEKIVQVSFQGLFLGRASELTPLLNQTFPEFGLDPKNCFQDPVVNCNELPFIKKECFEVPWIRSTLYFASKRTNNSIEFLVNNTVSQTKNYQKATSDFVKTPMQEEVWGMIKDLFLEEEKPMIILDPLGGKLDEISESELPFPHRKGNLFNIQYMVNWGDNSESISSKKIKWLRELYEKMKPFLSHCPRTAYLNYRDLDFGTNDGVYRYSKAKVWGAKYFNENFERLAKVKSKVDPGNFFRYEQSIPPFSVSTWSHPQFEK

SEQ ID NO: 74 - DNA, 1860 bp, synthetic construct
  misc_feature 1..1860: note = CBS94-A0A1S3XA06-full alpha factor-no signal peptide-strep Tag
  source 1..1860: mol_type = other DNA
  CDS 1..1860
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatggcccaacaagagttcttgcaatgtatctactctcactcctccgagaatattaagaagcacattcatttcccaaacagcccaacttactcttcgttgttggaatacgctcaaaagaatccaagatggctgaattcttctttcgcccatccaattttcattatcgctccaaagaacgaatccgaaatccagccaattatcttgtgctctcaaaagttcggcttgcaaatcagagttaagtctggtggtcatgattacgaaggtttgtctttcagatctgtcactaacacccaattcgttatcttggacttgatcaacttggacgatatcagaatcgacaagaacgaagaaactgcttggattcaaactggtgttactatgggtcagttgtactacgaaattgccaagaagtctaagatcttggcttttccaggtggtctatatccatctgttggttctggtggtttgatttcaggtggtggtattggtactttgatgagaaagtttggtttgtccgctgataacgtcattgatgctagagttatggatgtcaacggtagaatcttggacaaaaagtctatgggcgaagatatgttctgggctattagaggtggtggcggttcttcttttggtgttattttggcttggaagatcaagttggttagaatcccaccaaaggttactgttttcaccgttagaagaaagttggaaggtaacaccatcaacttgttgcaaagatggcaaaacgtttcccataagttcccaaaggatttgttcatgagagtcttgatccaaaacatgggtttcggtaacgaaaagatcgttcaagtttcattccagggtttgtttttgggtagagcttctgaattgacccctttgttgaatcaaactttcccagaattcggtttggacccaaaaaactgtttccaagatccagttgttaactgcaacgaattgcccttcattaagaaagaatgcttcgaagttccatggatcagatctaccttgtactttgcttctaagaggaccaacaactccattgaattcttggttaacaacaccgtttctcagaccaagaactaccaaaaagctacttccgatttcgttaagaccccaatgcaagaagaggtttggggtatgatcaaagacttgttcttggaagaagagaagcccatgattattttggaccctttaggtggtaagttggacgaaatttctgaatccgaattgccatttccacacagaaagggtaacttgttcaacatccagtacatggttaattggggtgacaactccgaatctatctcctccaaaaaaatcaaatggctgcgtgagttgtacgagaagatgaagccatttttgtctcattgtcctagaaccgcttacttgaactacagagatttggacttcggtactaatgatggtgtctacagatactctaaagccaaagtttggggtgctaagtacttcaacgaaaactttgaaagattggccaaggtcaagtctaaagttgatccaggtaatttcttcaggtacgaacaatctattccaccattctctgtttccacctggtcacatccacaatttgaaaag

SEQ ID NO: 75 - protein, 620 aa, synthetic construct
  REGION 1..620: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAMAQQEFLQCIYSHSSENIKKHIHFPNSPTYSSLLEYAQKNPRWLNSSFAHPIFIIAPKNESEIQPIILCSQKFGLQIRVKSGGHDYEGLSFRSVTNTQFVILDLINLDDIRIDKNEETAWIQTGVTMGQLYYEIAKKSKILAFPGGLYPSVGSGGLISGGGIGTLMRKFGLSADNVIDARVMDVNGRILDKKSMGEDMFWAIRGGGGSSFGVILAWKIKLVRIPPKVTVFTVRRKLEGNTINLLQRWQNVSHKFPKDLFMRVLIQNMGFGNEKIVQVSFQGLFLGRASELTPLLNQTFPEFGLDPKNCFQDPVVNCNELPFIKKECFEVPWIRSTLYFASKRTNNSIEFLVNNTVSQTKNYQKATSDFVKTPMQEEVWGMIKDLFLEEEKPMIILDPLGGKLDEISESELPFPHRKGNLFNIQYMVNWGDNSESISSKKIKWLRELYEKMKPFLSHCPRTAYLNYRDLDFGTNDGVYRYSKAKVWGAKYFNENFERLAKVKSKVDPGNFFRYEQSIPPFSVSTWSHPQFEK

SEQ ID NO: 76 - DNA, 1608 bp, synthetic construct
  misc_feature 1..1608: note = CBS99-A0A1S3BDA8
  source 1..1608: mol_type = other DNA
  CDS 1..1608
atgaagttctcctctaccttgattccactgtctttggtttttatcgttgtctcctctgcttctttgtgtgcttctgcttctccacaaaagtaccaaaatttcttgcagtgcttgtccgaacactcttctaaatcttacccaatctccaaggttgtctataccccaatcaattcctcttactcctctgttttggacttctccattaggaacttgttgttctctaaaccagaaacgccaaagcctttgttgattattactccatctcacgtgtcccatattcaagctgctgttatttgttctaagtcccatggcttgcaaatcagaactagatctggtggtcatgatttcgaaggtttgtcttatgttgcttacagaccattcatcgttgtcgacttgattaacttgagatccgttaaggttgaagtcgaaaacaatactgcttgggttgaatctggtgctactttgggtgagttgtattacagaattggtgaaaagtctaggaccttggcttttccagctggtgtttgtccaactgttggtataggtggtcatttttctggtggtggttatggtttgatgttgagaaaatttggtttggccgctgataacgtcattgatgcttatttggttgatgccaacggtaaggttttggacaaagaatctatgggcgaagatttgttctgggctattagaggtggtggtggcggttcttttggtatagttgttgcttggaagatcaagttggttccagttccagaaactgttactatttgctctaccgacagaaacttggaagaagatgccattaagttgatccacagatggcaatacgttggtaacaaattggacgagaatatctacttgggtatcattttgactggtggtaacgcttctactcaagctggtattactaatccaactgccagattcttcagcttgtttttgggtagagctgatgaattggttactaccttgtctactatcttcccagaattgggtttgatcaagcaagattgtgttgaagcttcttgggtcgaatccactttgattattccaattggtgttcagccaatcgaaaccttggaaccattattgaacagaactttgaccaccttggactccactaagattaagtctgattacatcaccgaacctattccagttgctgctattaaggatatctggcagagattgaaaacccaagacattgaaacctctcagttggtttttgttccatacggtggtagaatgtcccaaatctctgaatctgaaactccattctctcatagagccagatacttgttcaagatcggttatgttgtaggttggaaggaccaatctttgaaggctaagaagagacatatctcctggttgagagaagtttatgagtacatggctccattcgtttctaaatcaccaagagctgcttacaccaattacagagacttggatattggctccaacaacaaatacggtaagacctcttacaagagagcttctgtttggggtttgaagtatttcggtaacaacttcgatagactggtttacgttaagaccaaggttgatccatacgatttcttcagacacgaacaatctattcccaccttg

SEQ ID NO: 77 - protein, 536 aa, synthetic construct
  REGION 1..536: note = Synthetic Construct
MKFSSTLIPLSLVFIVVSSASLCASASPQKYQNFLQCLSEHSSKSYPISKVVYTPINSSYSSVLDFSIRNLLFSKPETPKPLLIITPSHVSHIQAAVICSKSHGLQIRTRSGGHDFEGLSYVAYRPFIVVDLINLRSVKVEVENNTAWVESGATLGELYYRIGEKSRTLAFPAGVCPTVGIGGHFSGGGYGLMLRKFGLAADNVIDAYLVDANGKVLDKESMGEDLFWAIRGGGGGSFGIVVAWKIKLVPVPETVTICSTDRNLEEDAIKLIHRWQYVGNKLDENIYLGIILTGGNASTQAGITNPTARFFSLFLGRADELVTTLSTIFPELGLIKQDCVEASWVESTLIIPIGVQPIETLEPLLNRTLTTLDSTKIKSDYITEPIPVAAIKDIWQRLKTQDIETSQLVFVPYGGRMSQISESETPFSHRARYLFKIGYVVGWKDQSLKAKKRHISWLREVYEYMAPFVSKSPRAAYTNYRDLDIGSNNKYGKTSYKRASVWGLKYFGNNFDRLVYVKTKVDPYDFFRHEQSIPTL

SEQ ID NO: 78 - DNA, 1536 bp, synthetic construct
  misc_feature 1..1536: note = CBS99-A0A1S3BDA8-no signal peptide
  source 1..1536: mol_type = other DNA
  CDS 1..1536
atggcttctccacaaaagtaccaaaatttcttgcagtgcttgtccgaacactcttctaaatcttacccaatctccaaggttgtctataccccaatcaattcctcttactcctctgttttggacttctccattaggaacttgttgttctctaaaccagaaacgccaaagcctttgttgattattactccatctcacgtgtcccatattcaagctgctgttatttgttctaagtcccatggcttgcaaatcagaactagatctggtggtcatgatttcgaaggtttgtcttatgttgcttacagaccattcatcgttgtcgacttgattaacttgagatccgttaaggttgaagtcgaaaacaatactgcttgggttgaatctggtgctactttgggtgagttgtattacagaattggtgaaaagtctaggaccttggcttttccagctggtgtttgtccaactgttggtataggtggtcatttttctggtggtggttatggtttgatgttgagaaaatttggtttggccgctgataacgtcattgatgcttatttggttgatgccaacggtaaggttttggacaaagaatctatgggcgaagatttgttctgggctattagaggtggtggtggcggttcttttggtatagttgttgcttggaagatcaagttggttccagttccagaaactgttactatttgctctaccgacagaaacttggaagaagatgccattaagttgatccacagatggcaatacgttggtaacaaattggacgagaatatctacttgggtatcattttgactggtggtaacgcttctactcaagctggtattactaatccaactgccagattcttcagcttgtttttgggtagagctgatgaattggttactaccttgtctactatcttcccagaattgggtttgatcaagcaagattgtgttgaagcttcttgggtcgaatccactttgattattccaattggtgttcagccaatcgaaaccttggaaccattattgaacagaactttgaccaccttggactccactaagattaagtctgattacatcaccgaacctattccagttgctgctattaaggatatctggcagagattgaaaacccaagacattgaaacctctcagttggtttttgttccatacggtggtagaatgtcccaaatctctgaatctgaaactccattctctcatagagccagatacttgttcaagatcggttatgttgtaggttggaaggaccaatctttgaaggctaagaagagacatatctcctggttgagagaagtttatgagtacatggctccattcgtttctaaatcaccaagagctgcttacaccaattacagagacttggatattggctccaacaacaaatacggtaagacctcttacaagagagcttctgtttggggtttgaagtatttcggtaacaacttcgatagactggtttacgttaagaccaaggttgatccatacgatttcttcagacacgaacaatctattcccaccttg

SEQ ID NO: 79 - protein, 512 aa, synthetic construct
  REGION 1..512: note = Synthetic Construct
MASPQKYQNFLQCLSEHSSKSYPISKVVYTPINSSYSSVLDFSIRNLLFSKPETPKPLLIITPSHVSHIQAAVICSKSHGLQIRTRSGGHDFEGLSYVAYRPFIVVDLINLRSVKVEVENNTAWVESGATLGELYYRIGEKSRTLAFPAGVCPTVGIGGHFSGGGYGLMLRKFGLAADNVIDAYLVDANGKVLDKESMGEDLFWAIRGGGGGSFGIVVAWKIKLVPVPETVTICSTDRNLEEDAIKLIHRWQYVGNKLDENIYLGIILTGGNASTQAGITNPTARFFSLFLGRADELVTTLSTIFPELGLIKQDCVEASWVESTLIIPIGVQPIETLEPLLNRTLTTLDSTKIKSDYITEPIPVAAIKDIWQRLKTQDIETSQLVFVPYGGRMSQISESETPFSHRARYLFKIGYVVGWKDQSLKAKKRHISWLREVYEYMAPFVSKSPRAAYTNYRDLDIGSNNKYGKTSYKRASVWGLKYFGNNFDRLVYVKTKVDPYDFFRHEQSIPTL

SEQ ID NO: 80 - DNA, 1560 bp, synthetic construct
  misc_feature 1..1560: note = CBS99-A0A1S3BDA8-no signal peptide-strep Tag
  source 1..1560: mol_type = other DNA
  CDS 1..1560
atggcttctccacaaaagtaccaaaatttcttgcagtgcttgtccgaacactcttctaaatcttacccaatctccaaggttgtctataccccaatcaattcctcttactcctctgttttggacttctccattaggaacttgttgttctctaaaccagaaacgccaaagcctttgttgattattactccatctcacgtgtcccatattcaagctgctgttatttgttctaagtcccatggcttgcaaatcagaactagatctggtggtcatgatttcgaaggtttgtcttatgttgcttacagaccattcatcgttgtcgacttgattaacttgagatccgttaaggttgaagtcgaaaacaatactgcttgggttgaatctggtgctactttgggtgagttgtattacagaattggtgaaaagtctaggaccttggcttttccagctggtgtttgtccaactgttggtataggtggtcatttttctggtggtggttatggtttgatgttgagaaaatttggtttggccgctgataacgtcattgatgcttatttggttgatgccaacggtaaggttttggacaaagaatctatgggcgaagatttgttctgggctattagaggtggtggtggcggttcttttggtatagttgttgcttggaagatcaagttggttccagttccagaaactgttactatttgctctaccgacagaaacttggaagaagatgccattaagttgatccacagatggcaatacgttggtaacaaattggacgagaatatctacttgggtatcattttgactggtggtaacgcttctactcaagctggtattactaatccaactgccagattcttcagcttgtttttgggtagagctgatgaattggttactaccttgtctactatcttcccagaattgggtttgatcaagcaagattgtgttgaagcttcttgggtcgaatccactttgattattccaattggtgttcagccaatcgaaaccttggaaccattattgaacagaactttgaccaccttggactccactaagattaagtctgattacatcaccgaacctattccagttgctgctattaaggatatctggcagagattgaaaacccaagacattgaaacctctcagttggtttttgttccatacggtggtagaatgtcccaaatctctgaatctgaaactccattctctcatagagccagatacttgttcaagatcggttatgttgtaggttggaaggaccaatctttgaaggctaagaagagacatatctcctggttgagagaagtttatgagtacatggctccattcgtttctaaatcaccaagagctgcttacaccaattacagagacttggatattggctccaacaacaaatacggtaagacctcttacaagagagcttctgtttggggtttgaagtatttcggtaacaacttcgatagactggtttacgttaagaccaaggttgatccatacgatttcttcagacacgaacaatctattcccaccttgtggtcacatccacaatttgaaaag

SEQ ID NO: 81 - protein, 520 aa, synthetic construct
  REGION 1..520: note = Synthetic Construct
MASPQKYQNFLQCLSEHSSKSYPISKVVYTPINSSYSSVLDFSIRNLLFSKPETPKPLLIITPSHVSHIQAAVICSKSHGLQIRTRSGGHDFEGLSYVAYRPFIVVDLINLRSVKVEVENNTAWVESGATLGELYYRIGEKSRTLAFPAGVCPTVGIGGHFSGGGYGLMLRKFGLAADNVIDAYLVDANGKVLDKESMGEDLFWAIRGGGGGSFGIVVAWKIKLVPVPETVTICSTDRNLEEDAIKLIHRWQYVGNKLDENIYLGIILTGGNASTQAGITNPTARFFSLFLGRADELVTTLSTIFPELGLIKQDCVEASWVESTLIIPIGVQPIETLEPLLNRTLTTLDSTKIKSDYITEPIPVAAIKDIWQRLKTQDIETSQLVFVPYGGRMSQISESETPFSHRARYLFKIGYVVGWKDQSLKAKKRHISWLREVYEYMAPFVSKSPRAAYTNYRDLDIGSNNKYGKTSYKRASVWGLKYFGNNFDRLVYVKTKVDPYDFFRHEQSIPTLWSHPQFEK

SEQ ID NO: 82 - DNA, 1617 bp, synthetic construct
  misc_feature 1..1617: note = CBS99-A0A1S3BDA8- truncated alpha factor-no signal peptide-strep Tag
  source 1..1617: mol_type = other DNA
  CDS 1..1617
atgagatttccttccatcttcacggctgtgctatttgcagcatcctccgcacttgcaatggcttctccacaaaagtaccaaaatttcttgcagtgcttgtccgaacactcttctaaatcttacccaatctccaaggttgtctataccccaatcaattcctcttactcctctgttttggacttctccattaggaacttgttgttctctaaaccagaaacgccaaagcctttgttgattattactccatctcacgtgtcccatattcaagctgctgttatttgttctaagtcccatggcttgcaaatcagaactagatctggtggtcatgatttcgaaggtttgtcttatgttgcttacagaccattcatcgttgtcgacttgattaacttgagatccgttaaggttgaagtcgaaaacaatactgcttgggttgaatctggtgctactttgggtgagttgtattacagaattggtgaaaagtctaggaccttggcttttccagctggtgtttgtccaactgttggtataggtggtcatttttctggtggtggttatggtttgatgttgagaaaatttggtttggccgctgataacgtcattgatgcttatttggttgatgccaacggtaaggttttggacaaagaatctatgggcgaagatttgttctgggctattagaggtggtggtggcggttcttttggtatagttgttgcttggaagatcaagttggttccagttccagaaactgttactatttgctctaccgacagaaacttggaagaagatgccattaagttgatccacagatggcaatacgttggtaacaaattggacgagaatatctacttgggtatcattttgactggtggtaacgcttctactcaagctggtattactaatccaactgccagattcttcagcttgtttttgggtagagctgatgaattggttactaccttgtctactatcttcccagaattgggtttgatcaagcaagattgtgttgaagcttcttgggtcgaatccactttgattattccaattggtgttcagccaatcgaaaccttggaaccattattgaacagaactttgaccaccttggactccactaagattaagtctgattacatcaccgaacctattccagttgctgctattaaggatatctggcagagattgaaaacccaagacattgaaacctctcagttggtttttgttccatacggtggtagaatgtcccaaatctctgaatctgaaactccattctctcatagagccagatacttgttcaagatcggttatgttgtaggttggaaggaccaatctttgaaggctaagaagagacatatctcctggttgagagaagtttatgagtacatggctccattcgtttctaaatcaccaagagctgcttacaccaattacagagacttggatattggctccaacaacaaatacggtaagacctcttacaagagagcttctgtttggggtttgaagtatttcggtaacaacttcgatagactggtttacgttaagaccaaggttgatccatacgatttcttcagacacgaacaatctattcccaccttgtggtcacatccacaatttgaaaag

SEQ ID NO: 83 - protein, 539 aa, synthetic construct
  REGION 1..539: note = Synthetic Construct
MRFPSIFTAVLFAASSALAMASPQKYQNFLQCLSEHSSKSYPISKVVYTPINSSYSSVLDFSIRNLLFSKPETPKPLLIITPSHVSHIQAAVICSKSHGLQIRTRSGGHDFEGLSYVAYRPFIVVDLINLRSVKVEVENNTAWVESGATLGELYYRIGEKSRTLAFPAGVCPTVGIGGHFSGGGYGLMLRKFGLAADNVIDAYLVDANGKVLDKESMGEDLFWAIRGGGGGSFGIVVAWKIKLVPVPETVTICSTDRNLEEDAIKLIHRWQYVGNKLDENIYLGIILTGGNASTQAGITNPTARFFSLFLGRADELVTTLSTIFPELGLIKQDCVEASWVESTLIIPIGVQPIETLEPLLNRTLTTLDSTKIKSDYITEPIPVAAIKDIWQRLKTQDIETSQLVFVPYGGRMSQISESETPFSHRARYLFKIGYVVGWKDQSLKAKKRHISWLREVYEYMAPFVSKSPRAAYTNYRDLDIGSNNKYGKTSYKRASVWGLKYFGNNFDRLVYVKTKVDPYDFFRHEQSIPTLWSHPQFEK

SEQ ID NO: 84 - DNA, 1827 bp, synthetic construct
  misc_feature 1..1827: note = CBS99-A0A1S3BDA8- full alpha factor -no signal peptide-strep Tag
  source 1..1827: mol_type = other DNA
  CDS 1..1827
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatggcttctccacaaaagtaccaaaatttcttgcagtgcttgtccgaacactcttctaaatcttacccaatctccaaggttgtctataccccaatcaattcctcttactcctctgttttggacttctccattaggaacttgttgttctctaaaccagaaacgccaaagcctttgttgattattactccatctcacgtgtcccatattcaagctgctgttatttgttctaagtcccatggcttgcaaatcagaactagatctggtggtcatgatttcgaaggtttgtcttatgttgcttacagaccattcatcgttgtcgacttgattaacttgagatccgttaaggttgaagtcgaaaacaatactgcttgggttgaatctggtgctactttgggtgagttgtattacagaattggtgaaaagtctaggaccttggcttttccagctggtgtttgtccaactgttggtataggtggtcatttttctggtggtggttatggtttgatgttgagaaaatttggtttggccgctgataacgtcattgatgcttatttggttgatgccaacggtaaggttttggacaaagaatctatgggcgaagatttgttctgggctattagaggtggtggtggcggttcttttggtatagttgttgcttggaagatcaagttggttccagttccagaaactgttactatttgctctaccgacagaaacttggaagaagatgccattaagttgatccacagatggcaatacgttggtaacaaattggacgagaatatctacttgggtatcattttgactggtggtaacgcttctactcaagctggtattactaatccaactgccagattcttcagcttgtttttgggtagagctgatgaattggttactaccttgtctactatcttcccagaattgggtttgatcaagcaagattgtgttgaagcttcttgggtcgaatccactttgattattccaattggtgttcagccaatcgaaaccttggaaccattattgaacagaactttgaccaccttggactccactaagattaagtctgattacatcaccgaacctattccagttgctgctattaaggatatctggcagagattgaaaacccaagacattgaaacctctcagttggtttttgttccatacggtggtagaatgtcccaaatctctgaatctgaaactccattctctcatagagccagatacttgttcaagatcggttatgttgtaggttggaaggaccaatctttgaaggctaagaagagacatatctcctggttgagagaagtttatgagtacatggctccattcgtttctaaatcaccaagagctgcttacaccaattacagagacttggatattggctccaacaacaaatacggtaagacctcttacaagagagcttctgtttggggtttgaagtatttcggtaacaacttcgatagactggtttacgttaagaccaaggttgatccatacgatttcttcagacacgaacaatctattcccaccttgtggtcacatccacaatttgaaaag

SEQ ID NO: 85 - protein, 609 aa, synthetic construct
  REGION 1..609: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAMASPQKYQNFLQCLSEHSSKSYPISKVVYTPINSSYSSVLDFSIRNLLFSKPETPKPLLIITPSHVSHIQAAVICSKSHGLQIRTRSGGHDFEGLSYVAYRPFIVVDLINLRSVKVEVENNTAWVESGATLGELYYRIGEKSRTLAFPAGVCPTVGIGGHFSGGGYGLMLRKFGLAADNVIDAYLVDANGKVLDKESMGEDLFWAIRGGGGGSFGIVVAWKIKLVPVPETVTICSTDRNLEEDAIKLIHRWQYVGNKLDENIYLGIILTGGNASTQAGITNPTARFFSLFLGRADELVTTLSTIFPELGLIKQDCVEASWVESTLIIPIGVQPIETLEPLLNRTLTTLDSTKIKSDYITEPIPVAAIKDIWQRLKTQDIETSQLVFVPYGGRMSQISESETPFSHRARYLFKIGYVVGWKDQSLKAKKRHISWLREVYEYMAPFVSKSPRAAYTNYRDLDIGSNNKYGKTSYKRASVWGLKYFGNNFDRLVYVKTKVDPYDFFRHEQSIPTLWSHPQFEK

SEQ ID NO: 86 - DNA, 1608 bp, synthetic construct
  misc_feature 1..1608: note = CBS124-A0A1U8FGM5
  source 1..1608: mol_type = other DNA
  CDS 1..1608
atgggtaacttgcaaatcatctgcgtcttgttgttctctttgttcttggctaagtgctactccagagaagatttcttgagatgcttgttcaagtcctccgaatctaacgttactcaaaacatctacaccccaaactcattgacctactcctctattttgaagtacgctcaaaaacactccagatgggttaattcttctcagccaaatttcattgcttctcccacaaaagaatccgaaatcaagccagttattttgtgcgctaagaagttgggtctgcaaatcaaaatcaaatccggtggtcatgattacgagggtatttcttacagatctgaattcccattcgtcatgttggacttgtccaatttgaacaagaccgaaatcgacttgaacgaagaaaccatttgggttcaagctggtgctactttgggtcaactatattacgctattgccaacagatccaacgttcatggttttccaggtggtatttgtttctctgttggtactggtggtttgatttctggtggtggtttaggtactatgatgagaaaatttggtttggccgctgataacgttgttgatgctagagttatggatgtcaacggtaacatcttggaccgtaaaaagatgaagaacgatttgttctgggcaattagaggtggtggtggcgcttcttttggtgttattttggcttggaaattgaagttggttagggttccagaaaaggttactgttttcaccgtctgcaaaaagttcgatgataaccagaacttgatcctgaagtgggaaaacattgctcatcaattgccagataagttggtcatcagagttgttatccaaaacgatggtacaggtaacgataagtacgtcgaaatgagatttcaagccttgtatttgggtccagttgatgaattgatccccttgttgaaagaaaagttcccagaattcgacttggaaaagaaggattgcttgcaagaaccagttggtgatagatctaacagatcctgcattaacaaagaatgcagagaaacctcttggatcggttctgtcttgtacttttacgaaagagaaaccacctcctcattggaggtcttattgaaagaatccattccaaagcagaagtcctactacaaaggtacttctgatttcgtcaagacccagatttctgaatctggttgggaaatggttgaaaggttgttgttggaagaagaatccccaagaattatcttggaaccattaggtggtaagttggacgaaatctccgaatcagaaattccatttccacacagaaaaggcaacttgtacaacatccaatacttggtttactggcacgacaacaacgaaaacgtttcctctcaaaaaattgcctggatgcgtaagctgtacaaagaaatggaaccatacgttgctaactctccaagaactgcttacttgaactacagagatttggacttcggttctaacgatgacaactactcttactccaaagctaaaatctggggcgaaaagtacttcaactacaactttgaaagactggccaaggttaagtctaaggttgatccaaacaacttcttcagaaccgaacaatctattcccttgtaccaaacctac

SEQ ID NO: 87 - protein, 536 aa, synthetic construct
  REGION 1..536: note = Synthetic Construct
MGNLQIICVLLFSLFLAKCYSREDFLRCLFKSSESNVTQNIYTPNSLTYSSILKYAQKHSRWVNSSQPNFIASPTKESEIKPVILCAKKLGLQIKIKSGGHDYEGISYRSEFPFVMLDLSNLNKTEIDLNEETIWVQAGATLGQLYYAIANRSNVHGFPGGICFSVGTGGLISGGGLGTMMRKFGLAADNVVDARVMDVNGNILDRKKMKNDLFWAIRGGGGASFGVILAWKLKLVRVPEKVTVFTVCKKFDDNQNLILKWENIAHQLPDKLVIRVVIQNDGTGNDKYVEMRFQALYLGPVDELIPLLKEKFPEFDLEKKDCLQEPVGDRSNRSCINKECRETSWIGSVLYFYERETTSSLEVLLKESIPKQKSYYKGTSDFVKTQISESGWEMVERLLLEEESPRIILEPLGGKLDEISESEIPFPHRKGNLYNIQYLVYWHDNNENVSSQKIAWMRKLYKEMEPYVANSPRTAYLNYRDLDFGSNDDNYSYSKAKIWGEKYFNYNFERLAKVKSKVDPNNFFRTEQSIPLYQTY

SEQ ID NO: 88 - DNA, 1551 bp, synthetic construct
  misc_feature 1..1551: note = CBS124-A0A1U8FGM5-no signal peptide
  source 1..1551: mol_type = other DNA
  CDS 1..1551
atgtccagagaagatttcttgagatgcttgttcaagtcctccgaatctaacgttactcaaaacatctacaccccaaactcattgacctactcctctattttgaagtacgctcaaaaacactccagatgggttaattcttctcagccaaatttcattgcttctcccacaaaagaatccgaaatcaagccagttattttgtgcgctaagaagttgggtctgcaaatcaaaatcaaatccggtggtcatgattacgagggtatttcttacagatctgaattcccattcgtcatgttggacttgtccaatttgaacaagaccgaaatcgacttgaacgaagaaaccatttgggttcaagctggtgctactttgggtcaactatattacgctattgccaacagatccaacgttcatggttttccaggtggtatttgtttctctgttggtactggtggtttgatttctggtggtggtttaggtactatgatgagaaaatttggtttggccgctgataacgttgttgatgctagagttatggatgtcaacggtaacatcttggaccgtaaaaagatgaagaacgatttgttctgggcaattagaggtggtggtggcgcttcttttggtgttattttggcttggaaattgaagttggttagggttccagaaaaggttactgttttcaccgtctgcaaaaagttcgatgataaccagaacttgatcctgaagtgggaaaacattgctcatcaattgccagataagttggtcatcagagttgttatccaaaacgatggtacaggtaacgataagtacgtcgaaatgagatttcaagccttgtatttgggtccagttgatgaattgatccccttgttgaaagaaaagttcccagaattcgacttggaaaagaaggattgcttgcaagaaccagttggtgatagatctaacagatcctgcattaacaaagaatgcagagaaacctcttggatcggttctgtcttgtacttttacgaaagagaaaccacctcctcattggaggtcttattgaaagaatccattccaaagcagaagtcctactacaaaggtacttctgatttcgtcaagacccagatttctgaatctggttgggaaatggttgaaaggttgttgttggaagaagaatccccaagaattatcttggaaccattaggtggtaagttggacgaaatctccgaatcagaaattccatttccacacagaaaaggcaacttgtacaacatccaatacttggtttactggcacgacaacaacgaaaacgtttcctctcaaaaaattgcctggatgcgtaagctgtacaaagaaatggaaccatacgttgctaactctccaagaactgcttacttgaactacagagatttggacttcggttctaacgatgacaactactcttactccaaagctaaaatctggggcgaaaagtacttcaactacaactttgaaagactggccaaggttaagtctaaggttgatccaaacaacttcttcagaaccgaacaatctattcccttgtaccaaacctac

SEQ ID NO: 89 - protein, 517 aa, synthetic construct
  REGION 1..517: note = Synthetic Construct
MSREDFLRCLFKSSESNVTQNIYTPNSLTYSSILKYAQKHSRWVNSSQPNFIASPTKESEIKPVILCAKKLGLQIKIKSGGHDYEGISYRSEFPFVMLDLSNLNKTEIDLNEETIWVQAGATLGQLYYAIANRSNVHGFPGGICFSVGTGGLISGGGLGTMMRKFGLAADNVVDARVMDVNGNILDRKKMKNDLFWAIRGGGGASFGVILAWKLKLVRVPEKVTVFTVCKKFDDNQNLILKWENIAHQLPDKLVIRVVIQNDGTGNDKYVEMRFQALYLGPVDELIPLLKEKFPEFDLEKKDCLQEPVGDRSNRSCINKECRETSWIGSVLYFYERETTSSLEVLLKESIPKQKSYYKGTSDFVKTQISESGWEMVERLLLEEESPRIILEPLGGKLDEISESEIPFPHRKGNLYNIQYLVYWHDNNENVSSQKIAWMRKLYKEMEPYVANSPRTAYLNYRDLDFGSNDDNYSYSKAKIWGEKYFNYNFERLAKVKSKVDPNNFFRTEQSIPLYQTY

SEQ ID NO: 90 - DNA, 1575 bp, synthetic construct
  misc_feature 1..1575: note = CBS124-A0A1U8FGM5-no signal peptide-strep Tag
  source 1..1575: mol_type = other DNA
  CDS 1..1575
atgtccagagaagatttcttgagatgcttgttcaagtcctccgaatctaacgttactcaaaacatctacaccccaaactcattgacctactcctctattttgaagtacgctcaaaaacactccagatgggttaattcttctcagccaaatttcattgcttctcccacaaaagaatccgaaatcaagccagttattttgtgcgctaagaagttgggtctgcaaatcaaaatcaaatccggtggtcatgattacgagggtatttcttacagatctgaattcccattcgtcatgttggacttgtccaatttgaacaagaccgaaatcgacttgaacgaagaaaccatttgggttcaagctggtgctactttgggtcaactatattacgctattgccaacagatccaacgttcatggttttccaggtggtatttgtttctctgttggtactggtggtttgatttctggtggtggtttaggtactatgatgagaaaatttggtttggccgctgataacgttgttgatgctagagttatggatgtcaacggtaacatcttggaccgtaaaaagatgaagaacgatttgttctgggcaattagaggtggtggtggcgcttcttttggtgttattttggcttggaaattgaagttggttagggttccagaaaaggttactgttttcaccgtctgcaaaaagttcgatgataaccagaacttgatcctgaagtgggaaaacattgctcatcaattgccagataagttggtcatcagagttgttatccaaaacgatggtacaggtaacgataagtacgtcgaaatgagatttcaagccttgtatttgggtccagttgatgaattgatccccttgttgaaagaaaagttcccagaattcgacttggaaaagaaggattgcttgcaagaaccagttggtgatagatctaacagatcctgcattaacaaagaatgcagagaaacctcttggatcggttctgtcttgtacttttacgaaagagaaaccacctcctcattggaggtcttattgaaagaatccattccaaagcagaagtcctactacaaaggtacttctgatttcgtcaagacccagatttctgaatctggttgggaaatggttgaaaggttgttgttggaagaagaatccccaagaattatcttggaaccattaggtggtaagttggacgaaatctccgaatcagaaattccatttccacacagaaaaggcaacttgtacaacatccaatacttggtttactggcacgacaacaacgaaaacgtttcctctcaaaaaattgcctggatgcgtaagctgtacaaagaaatggaaccatacgttgctaactctccaagaactgcttacttgaactacagagatttggacttcggttctaacgatgacaactactcttactccaaagctaaaatctggggcgaaaagtacttcaactacaactttgaaagactggccaaggttaagtctaaggttgatccaaacaacttcttcagaaccgaacaatctattcccttgtaccaaacctactggtcacatccacaatttgaaaag

SEQ ID NO: 91 - protein, 525 aa, synthetic construct
  REGION 1..525: note = Synthetic Construct
MSREDFLRCLFKSSESNVTQNIYTPNSLTYSSILKYAQKHSRWVNSSQPNFIASPTKESEIKPVILCAKKLGLQIKIKSGGHDYEGISYRSEFPFVMLDLSNLNKTEIDLNEETIWVQAGATLGQLYYAIANRSNVHGFPGGICFSVGTGGLISGGGLGTMMRKFGLAADNVVDARVMDVNGNILDRKKMKNDLFWAIRGGGGASFGVILAWKLKLVRVPEKVTVFTVCKKFDDNQNLILKWENIAHQLPDKLVIRVVIQNDGTGNDKYVEMRFQALYLGPVDELIPLLKEKFPEFDLEKKDCLQEPVGDRSNRSCINKECRETSWIGSVLYFYERETTSSLEVLLKESIPKQKSYYKGTSDFVKTQISESGWEMVERLLLEEESPRIILEPLGGKLDEISESEIPFPHRKGNLYNIQYLVYWHDNNENVSSQKIAWMRKLYKEMEPYVANSPRTAYLNYRDLDFGSNDDNYSYSKAKIWGEKYFNYNFERLAKVKSKVDPNNFFRTEQSIPLYQTYWSHPQFEK

SEQ ID NO: 92 - DNA, 1632 bp, synthetic construct
  misc_feature 1..1632: note = CBS124-A0A1U8FGM5- truncated alpha factor - no signal peptide-strep Tag
  source 1..1632: mol_type = other DNA
  CDS 1..1632
atgagatttccttccatcttcacggctgtgctatttgcagcatcctccgcacttgcaatgtccagagaagatttcttgagatgcttgttcaagtcctccgaatctaacgttactcaaaacatctacaccccaaactcattgacctactcctctattttgaagtacgctcaaaaacactccagatgggttaattcttctcagccaaatttcattgcttctcccacaaaagaatccgaaatcaagccagttattttgtgcgctaagaagttgggtctgcaaatcaaaatcaaatccggtggtcatgattacgagggtatttcttacagatctgaattcccattcgtcatgttggacttgtccaatttgaacaagaccgaaatcgacttgaacgaagaaaccatttgggttcaagctggtgctactttgggtcaactatattacgctattgccaacagatccaacgttcatggttttccaggtggtatttgtttctctgttggtactggtggtttgatttctggtggtggtttaggtactatgatgagaaaatttggtttggccgctgataacgttgttgatgctagagttatggatgtcaacggtaacatcttggaccgtaaaaagatgaagaacgatttgttctgggcaattagaggtggtggtggcgcttcttttggtgttattttggcttggaaattgaagttggttagggttccagaaaaggttactgttttcaccgtctgcaaaaagttcgatgataaccagaacttgatcctgaagtgggaaaacattgctcatcaattgccagataagttggtcatcagagttgttatccaaaacgatggtacaggtaacgataagtacgtcgaaatgagatttcaagccttgtatttgggtccagttgatgaattgatccccttgttgaaagaaaagttcccagaattcgacttggaaaagaaggattgcttgcaagaaccagttggtgatagatctaacagatcctgcattaacaaagaatgcagagaaacctcttggatcggttctgtcttgtacttttacgaaagagaaaccacctcctcattggaggtcttattgaaagaatccattccaaagcagaagtcctactacaaaggtacttctgatttcgtcaagacccagatttctgaatctggttgggaaatggttgaaaggttgttgttggaagaagaatccccaagaattatcttggaaccattaggtggtaagttggacgaaatctccgaatcagaaattccatttccacacagaaaaggcaacttgtacaacatccaatacttggtttactggcacgacaacaacgaaaacgtttcctctcaaaaaattgcctggatgcgtaagctgtacaaagaaatggaaccatacgttgctaactctccaagaactgcttacttgaactacagagatttggacttcggttctaacgatgacaactactcttactccaaagctaaaatctggggcgaaaagtacttcaactacaactttgaaagactggccaaggttaagtctaaggttgatccaaacaacttcttcagaaccgaacaatctattcccttgtaccaaacctactggtcacatccacaatttgaaaag

SEQ ID NO: 93 - protein, 544 aa, synthetic construct
  REGION 1..544: note = Synthetic Construct
MRFPSIFTAVLFAASSALAMSREDFLRCLFKSSESNVTQNIYTPNSLTYSSILKYAQKHSRWVNSSQPNFIASPTKESEIKPVILCAKKLGLQIKIKSGGHDYEGISYRSEFPFVMLDLSNLNKTEIDLNEETIWVQAGATLGQLYYAIANRSNVHGFPGGICFSVGTGGLISGGGLGTMMRKFGLAADNVVDARVMDVNGNILDRKKMKNDLFWAIRGGGGASFGVILAWKLKLVRVPEKVTVFTVCKKFDDNQNLILKWENIAHQLPDKLVIRVVIQNDGTGNDKYVEMRFQALYLGPVDELIPLLKEKFPEFDLEKKDCLQEPVGDRSNRSCINKECRETSWIGSVLYFYERETTSSLEVLLKESIPKQKSYYKGTSDFVKTQISESGWEMVERLLLEEESPRIILEPLGGKLDEISESEIPFPHRKGNLYNIQYLVYWHDNNENVSSQKIAWMRKLYKEMEPYVANSPRTAYLNYRDLDFGSNDDNYSYSKAKIWGEKYFNYNFERLAKVKSKVDPNNFFRTEQSIPLYQTYWSHPQFEK

SEQ ID NO: 94 - DNA, 1842 bp, synthetic construct
  misc_feature 1..1842: note = CBS124-A0A1U8FGM5- full alpha factor -no signal peptide-strep Tag
  source 1..1842: mol_type = other DNA
  CDS 1..1842
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatgtccagagaagatttcttgagatgcttgttcaagtcctccgaatctaacgttactcaaaacatctacaccccaaactcattgacctactcctctattttgaagtacgctcaaaaacactccagatgggttaattcttctcagccaaatttcattgcttctcccacaaaagaatccgaaatcaagccagttattttgtgcgctaagaagttgggtctgcaaatcaaaatcaaatccggtggtcatgattacgagggtatttcttacagatctgaattcccattcgtcatgttggacttgtccaatttgaacaagaccgaaatcgacttgaacgaagaaaccatttgggttcaagctggtgctactttgggtcaactatattacgctattgccaacagatccaacgttcatggttttccaggtggtatttgtttctctgttggtactggtggtttgatttctggtggtggtttaggtactatgatgagaaaatttggtttggccgctgataacgttgttgatgctagagttatggatgtcaacggtaacatcttggaccgtaaaaagatgaagaacgatttgttctgggcaattagaggtggtggtggcgcttcttttggtgttattttggcttggaaattgaagttggttagggttccagaaaaggttactgttttcaccgtctgcaaaaagttcgatgataaccagaacttgatcctgaagtgggaaaacattgctcatcaattgccagataagttggtcatcagagttgttatccaaaacgatggtacaggtaacgataagtacgtcgaaatgagatttcaagccttgtatttgggtccagttgatgaattgatccccttgttgaaagaaaagttcccagaattcgacttggaaaagaaggattgcttgcaagaaccagttggtgatagatctaacagatcctgcattaacaaagaatgcagagaaacctcttggatcggttctgtcttgtacttttacgaaagagaaaccacctcctcattggaggtcttattgaaagaatccattccaaagcagaagtcctactacaaaggtacttctgatttcgtcaagacccagatttctgaatctggttgggaaatggttgaaaggttgttgttggaagaagaatccccaagaattatcttggaaccattaggtggtaagttggacgaaatctccgaatcagaaattccatttccacacagaaaaggcaacttgtacaacatccaatacttggtttactggcacgacaacaacgaaaacgtttcctctcaaaaaattgcctggatgcgtaagctgtacaaagaaatggaaccatacgttgctaactctccaagaactgcttacttgaactacagagatttggacttcggttctaacgatgacaactactcttactccaaagctaaaatctggggcgaaaagtacttcaactacaactttgaaagactggccaaggttaagtctaaggttgatccaaacaacttcttcagaaccgaacaatctattcccttgtaccaaacctactggtcacatccacaatttgaaaag

SEQ ID NO: 95 - protein, 614 aa, synthetic construct
  REGION 1..614: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAMSREDFLRCLFKSSESNVTQNIYTPNSLTYSSILKYAQKHSRWVNSSQPNFIASPTKESEIKPVILCAKKLGLQIKIKSGGHDYEGISYRSEFPFVMLDLSNLNKTEIDLNEETIWVQAGATLGQLYYAIANRSNVHGFPGGICFSVGTGGLISGGGLGTMMRKFGLAADNVVDARVMDVNGNILDRKKMKNDLFWAIRGGGGASFGVILAWKLKLVRVPEKVTVFTVCKKFDDNQNLILKWENIAHQLPDKLVIRVVIQNDGTGNDKYVEMRFQALYLGPVDELIPLLKEKFPEFDLEKKDCLQEPVGDRSNRSCINKECRETSWIGSVLYFYERETTSSLEVLLKESIPKQKSYYKGTSDFVKTQISESGWEMVERLLLEEESPRIILEPLGGKLDEISESEIPFPHRKGNLYNIQYLVYWHDNNENVSSQKIAWMRKLYKEMEPYVANSPRTAYLNYRDLDFGSNDDNYSYSKAKIWGEKYFNYNFERLAKVKSKVDPNNFFRTEQSIPLYQTYWSHPQFEK

SEQ ID NO: 96 - DNA, 1641 bp, synthetic construct
  misc_feature 1..1641: note = CBS133-A0A1U8MHW2
  source 1..1641: mol_type = other DNA
  CDS 1..1641
atgttcccattcttgttcgtcgtcttgttgtctttgtcttggagaatcaatgcttctgctttgccagttcaagaattcctacattgcttgtctttgagatccccaaactcttctattgccaagttggtctatacccagaacaactcttcttactcctctgttttgaaatccgctgctcaaaacttcagattcactactccatctactccaactccattggctatagttactccattgcatgcctctcatattcaagctgctgtttactgttgtagaaaacacggtttacaagtcagaactagatctggtggtcatgattttgaaggtttgtcttacacttccgcttacaaggttccattcgttgttatcgatttggtcaacttgagatccgttagagttaacgttgaaaaagctactgcttgggttgaatctggtgctactgttggtgaattatacgacgaaattgccagaaagtctaagaccttgggttttccagctggtatttgtccaactttaggtgttggtggtcacttgtctggtggtggttatggtttgttgtttagaaagtatggtttgggtgccgataacgtcattgatgctagattgattgatgtcaagggtcgtatcttggacaaaaagtctatgggtgaagatttgttttgggccattagaggtggtggtggcggtacttttggtatagttttggcttggaaattgaagttggttgctgttccagctaacgttactgttttcactgttagaaagactttggaacaaaacgccactaagttggttcatagatggcaatctattgcccacaagttcccaaaagaaatgcacttgtctattgctattgctagggctaactcttccgaagatgaagaaaagatgactatccaagcttctttcacctctactttcttgggttccattgatgaattgatcccattgatggaagaaaagttcccagaattgggtttagtcaaagaagattgcctagaaatgtcttgggctgagtctaacttgtacagctttttgttcttgctgagatctccatctgaaaccctgttgaatagaaaccagaagtcctctatctccaaaacctttttcaaggccaagtccgattttgtcaaacagccaattccagaatctgctttcgaaggtttatggtccaagttttatgaagatgaggctaagtccgccatcatggtttttgttgcttttggtggtaagatggacgaaatcttggaaactgaatacccatatccacatagagccggtaacttatacactatcttgtacactgttgactgggaagagaaagagaacatcaactctgaaaagttcatgtcctggatgagaagagtctacaattacatgactccctacgtttctaagtctccaagagaagcttacatcaactacagagatttggacatcggtacaaacaagatcaacaacagaaagagatcttacgctaaggctagagtatggggtgttaagtactttaagaacaacttcaacaggctggttaacgtcaagactatagttgacccacagaatttcttcagacacgaacaatctattccaccactattcttgtcctggaaaagatccgatcataagaagcacttgtcctcc

SEQ ID NO: 97 - protein, 547 aa, synthetic construct
  REGION 1..547: note = Synthetic Construct
MFPFLFVVLLSLSWRINASALPVQEFLHCLSLRSPNSSIAKLVYTQNNSSYSSVLKSAAQNFRFTTPSTPTPLAIVTPLHASHIQAAVYCCRKHGLQVRTRSGGHDFEGLSYTSAYKVPFVVIDLVNLRSVRVNVEKATAWVESGATVGELYDEIARKSKTLGFPAGICPTLGVGGHLSGGGYGLLFRKYGLGADNVIDARLIDVKGRILDKKSMGEDLFWAIRGGGGGTFGIVLAWKLKLVAVPANVTVFTVRKTLEQNATKLVHRWQSIAHKFPKEMHLSIAIARANSSEDEEKMTIQASFTSTFLGSIDELIPLMEEKFPELGLVKEDCLEMSWAESNLYSFLFLLRSPSETLLNRNQKSSISKTFFKAKSDFVKQPIPESAFEGLWSKFYEDEAKSAIMVFVAFGGKMDEILETEYPYPHRAGNLYTILYTVDWEEKENINSEKFMSWMRRVYNYMTPYVSKSPREAYINYRDLDIGTNKINNRKRSYAKARVWGVKYFKNNFNRLVNVKTIVDPQNFFRHEQSIPPLFLSWKRSDHKKHLSS

SEQ ID NO: 98 - DNA, 1587 bp, synthetic construct
  misc_feature 1..1587: note = CBS133-A0A1U8MHW2- no signal peptide
  source 1..1587: mol_type = other DNA
  CDS 1..1587
atggctttgccagttcaagaattcctacattgcttgtctttgagatccccaaactcttctattgccaagttggtctatacccagaacaactcttcttactcctctgttttgaaatccgctgctcaaaacttcagattcactactccatctactccaactccattggctatagttactccattgcatgcctctcatattcaagctgctgtttactgttgtagaaaacacggtttacaagtcagaactagatctggtggtcatgattttgaaggtttgtcttacacttccgcttacaaggttccattcgttgttatcgatttggtcaacttgagatccgttagagttaacgttgaaaaagctactgcttgggttgaatctggtgctactgttggtgaattatacgacgaaattgccagaaagtctaagaccttgggttttccagctggtatttgtccaactttaggtgttggtggtcacttgtctggtggtggttatggtttgttgtttagaaagtatggtttgggtgccgataacgtcattgatgctagattgattgatgtcaagggtcgtatcttggacaaaaagtctatgggtgaagatttgttttgggccattagaggtggtggtggcggtacttttggtatagttttggcttggaaattgaagttggttgctgttccagctaacgttactgttttcactgttagaaagactttggaacaaaacgccactaagttggttcatagatggcaatctattgcccacaagttcccaaaagaaatgcacttgtctattgctattgctagggctaactcttccgaagatgaagaaaagatgactatccaagcttctttcacctctactttcttgggttccattgatgaattgatcccattgatggaagaaaagttcccagaattgggtttagtcaaagaagattgcctagaaatgtcttgggctgagtctaacttgtacagctttttgttcttgctgagatctccatctgaaaccctgttgaatagaaaccagaagtcctctatctccaaaacctttttcaaggccaagtccgattttgtcaaacagccaattccagaatctgctttcgaaggtttatggtccaagttttatgaagatgaggctaagtccgccatcatggtttttgttgcttttggtggtaagatggacgaaatcttggaaactgaatacccatatccacatagagccggtaacttatacactatcttgtacactgttgactgggaagagaaagagaacatcaactctgaaaagttcatgtcctggatgagaagagtctacaattacatgactccctacgtttctaagtctccaagagaagcttacatcaactacagagatttggacatcggtacaaacaagatcaacaacagaaagagatcttacgctaaggctagagtatggggtgttaagtactttaagaacaacttcaacaggctggttaacgtcaagactatagttgacccacagaatttcttcagacacgaacaatctattccaccactattcttgtcctggaaaagatccgatcataagaagcacttgtcctcc

SEQ ID NO: 99 - protein, 529 aa, synthetic construct
  REGION 1..529: note = Synthetic Construct
MALPVQEFLHCLSLRSPNSSIAKLVYTQNNSSYSSVLKSAAQNFRFTTPSTPTPLAIVTPLHASHIQAAVYCCRKHGLQVRTRSGGHDFEGLSYTSAYKVPFVVIDLVNLRSVRVNVEKATAWVESGATVGELYDEIARKSKTLGFPAGICPTLGVGGHLSGGGYGLLFRKYGLGADNVIDARLIDVKGRILDKKSMGEDLFWAIRGGGGGTFGIVLAWKLKLVAVPANVTVFTVRKTLEQNATKLVHRWQSIAHKFPKEMHLSIAIARANSSEDEEKMTIQASFTSTFLGSIDELIPLMEEKFPELGLVKEDCLEMSWAESNLYSFLFLLRSPSETLLNRNQKSSISKTFFKAKSDFVKQPIPESAFEGLWSKFYEDEAKSAIMVFVAFGGKMDEILETEYPYPHRAGNLYTILYTVDWEEKENINSEKFMSWMRRVYNYMTPYVSKSPREAYINYRDLDIGTNKINNRKRSYAKARVWGVKYFKNNFNRLVNVKTIVDPQNFFRHEQSIPPLFLSWKRSDHKKHLSS

SEQ ID NO: 100 - DNA, 1611 bp, synthetic construct
  misc_feature 1..1611: note = CBS133-A0A1U8MHW2- no signal peptide-strep Tag
  source 1..1611: mol_type = other DNA
  CDS 1..1611
atggctttgccagttcaagaattcctacattgcttgtctttgagatccccaaactcttctattgccaagttggtctatacccagaacaactcttcttactcctctgttttgaaatccgctgctcaaaacttcagattcactactccatctactccaactccattggctatagttactccattgcatgcctctcatattcaagctgctgtttactgttgtagaaaacacggtttacaagtcagaactagatctggtggtcatgattttgaaggtttgtcttacacttccgcttacaaggttccattcgttgttatcgatttggtcaacttgagatccgttagagttaacgttgaaaaagctactgcttgggttgaatctggtgctactgttggtgaattatacgacgaaattgccagaaagtctaagaccttgggttttccagctggtatttgtccaactttaggtgttggtggtcacttgtctggtggtggttatggtttgttgtttagaaagtatggtttgggtgccgataacgtcattgatgctagattgattgatgtcaagggtcgtatcttggacaaaaagtctatgggtgaagatttgttttgggccattagaggtggtggtggcggtacttttggtatagttttggcttggaaattgaagttggttgctgttccagctaacgttactgttttcactgttagaaagactttggaacaaaacgccactaagttggttcatagatggcaatctattgcccacaagttcccaaaagaaatgcacttgtctattgctattgctagggctaactcttccgaagatgaagaaaagatgactatccaagcttctttcacctctactttcttgggttccattgatgaattgatcccattgatggaagaaaagttcccagaattgggtttagtcaaagaagattgcctagaaatgtcttgggctgagtctaacttgtacagctttttgttcttgctgagatctccatctgaaaccctgttgaatagaaaccagaagtcctctatctccaaaacctttttcaaggccaagtccgattttgtcaaacagccaattccagaatctgctttcgaaggtttatggtccaagttttatgaagatgaggctaagtccgccatcatggtttttgttgcttttggtggtaagatggacgaaatcttggaaactgaatacccatatccacatagagccggtaacttatacactatcttgtacactgttgactgggaagagaaagagaacatcaactctgaaaagttcatgtcctggatgagaagagtctacaattacatgactccctacgtttctaagtctccaagagaagcttacatcaactacagagatttggacatcggtacaaacaagatcaacaacagaaagagatcttacgctaaggctagagtatggggtgttaagtactttaagaacaacttcaacaggctggttaacgtcaagactatagttgacccacagaatttcttcagacacgaacaatctattccaccactattcttgtcctggaaaagatccgatcataagaagcacttgtcctcctggtcacatccacaatttgaaaag

SEQ ID NO: 101 - protein, 537 aa, synthetic construct
  REGION 1..537: note = Synthetic Construct
MALPVQEFLHCLSLRSPNSSIAKLVYTQNNSSYSSVLKSAAQNFRFTTPSTPTPLAIVTPLHASHIQAAVYCCRKHGLQVRTRSGGHDFEGLSYTSAYKVPFVVIDLVNLRSVRVNVEKATAWVESGATVGELYDEIARKSKTLGFPAGICPTLGVGGHLSGGGYGLLFRKYGLGADNVIDARLIDVKGRILDKKSMGEDLFWAIRGGGGGTFGIVLAWKLKLVAVPANVTVFTVRKTLEQNATKLVHRWQSIAHKFPKEMHLSIAIARANSSEDEEKMTIQASFTSTFLGSIDELIPLMEEKFPELGLVKEDCLEMSWAESNLYSFLFLLRSPSETLLNRNQKSSISKTFFKAKSDFVKQPIPESAFEGLWSKFYEDEAKSAIMVFVAFGGKMDEILETEYPYPHRAGNLYTILYTVDWEEKENINSEKFMSWMRRVYNYMTPYVSKSPREAYINYRDLDIGTNKINNRKRSYAKARVWGVKYFKNNFNRLVNVKTIVDPQNFFRHEQSIPPLFLSWKRSDHKKHLSSWSHPQFEK

SEQ ID NO: 102 - DNA, 1668 bp, synthetic construct
  misc_feature 1..1668: note = CBS133-A0A1U8MHW2-truncated alpha factor - no signal peptide-strep Tag
  source 1..1668: mol_type = other DNA
  CDS 1..1668
atgagatttccttccatcttcacggctgtgctatttgcagcatcctccgcacttgcaatggctttgccagttcaagaattcctacattgcttgtctttgagatccccaaactcttctattgccaagttggtctatacccagaacaactcttcttactcctctgttttgaaatccgctgctcaaaacttcagattcactactccatctactccaactccattggctatagttactccattgcatgcctctcatattcaagctgctgtttactgttgtagaaaacacggtttacaagtcagaactagatctggtggtcatgattttgaaggtttgtcttacacttccgcttacaaggttccattcgttgttatcgatttggtcaacttgagatccgttagagttaacgttgaaaaagctactgcttgggttgaatctggtgctactgttggtgaattatacgacgaaattgccagaaagtctaagaccttgggttttccagctggtatttgtccaactttaggtgttggtggtcacttgtctggtggtggttatggtttgttgtttagaaagtatggtttgggtgccgataacgtcattgatgctagattgattgatgtcaagggtcgtatcttggacaaaaagtctatgggtgaagatttgttttgggccattagaggtggtggtggcggtacttttggtatagttttggcttggaaattgaagttggttgctgttccagctaacgttactgttttcactgttagaaagactttggaacaaaacgccactaagttggttcatagatggcaatctattgcccacaagttcccaaaagaaatgcacttgtctattgctattgctagggctaactcttccgaagatgaagaaaagatgactatccaagcttctttcacctctactttcttgggttccattgatgaattgatcccattgatggaagaaaagttcccagaattgggtttagtcaaagaagattgcctagaaatgtcttgggctgagtctaacttgtacagctttttgttcttgctgagatctccatctgaaaccctgttgaatagaaaccagaagtcctctatctccaaaacctttttcaaggccaagtccgattttgtcaaacagccaattccagaatctgctttcgaaggtttatggtccaagttttatgaagatgaggctaagtccgccatcatggtttttgttgcttttggtggtaagatggacgaaatcttggaaactgaatacccatatccacatagagccggtaacttatacactatcttgtacactgttgactgggaagagaaagagaacatcaactctgaaaagttcatgtcctggatgagaagagtctacaattacatgactccctacgtttctaagtctccaagagaagcttacatcaactacagagatttggacatcggtacaaacaagatcaacaacagaaagagatcttacgctaaggctagagtatggggtgttaagtactttaagaacaacttcaacaggctggttaacgtcaagactatagttgacccacagaatttcttcagacacgaacaatctattccaccactattcttgtcctggaaaagatccgatcataagaagcacttgtcctcctggtcacatccacaatttgaaaag

SEQ ID NO: 103 - protein, 556 aa, synthetic construct
  REGION 1..556: note = Synthetic Construct
MRFPSIFTAVLFAASSALAMALPVQEFLHCLSLRSPNSSIAKLVYTQNNSSYSSVLKSAAQNFRFTTPSTPTPLAIVTPLHASHIQAAVYCCRKHGLQVRTRSGGHDFEGLSYTSAYKVPFVVIDLVNLRSVRVNVEKATAWVESGATVGELYDEIARKSKTLGFPAGICPTLGVGGHLSGGGYGLLFRKYGLGADNVIDARLIDVKGRILDKKSMGEDLFWAIRGGGGGTFGIVLAWKLKLVAVPANVTVFTVRKTLEQNATKLVHRWQSIAHKFPKEMHLSIAIARANSSEDEEKMTIQASFTSTFLGSIDELIPLMEEKFPELGLVKEDCLEMSWAESNLYSFLFLLRSPSETLLNRNQKSSISKTFFKAKSDFVKQPIPESAFEGLWSKFYEDEAKSAIMVFVAFGGKMDEILETEYPYPHRAGNLYTILYTVDWEEKENINSEKFMSWMRRVYNYMTPYVSKSPREAYINYRDLDIGTNKINNRKRSYAKARVWGVKYFKNNFNRLVNVKTIVDPQNFFRHEQSIPPLFLSWKRSDHKKHLSSWSHPQFEK

SEQ ID NO: 104 - DNA, 1878 bp, synthetic construct
  misc_feature 1..1878: note = CBS133-A0A1U8MHW2-full alpha factor -no signal peptide-strep Tag
  source 1..1878: mol_type = other DNA
  CDS 1..1878
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatggctttgccagttcaagaattcctacattgcttgtctttgagatccccaaactcttctattgccaagttggtctatacccagaacaactcttcttactcctctgttttgaaatccgctgctcaaaacttcagattcactactccatctactccaactccattggctatagttactccattgcatgcctctcatattcaagctgctgtttactgttgtagaaaacacggtttacaagtcagaactagatctggtggtcatgattttgaaggtttgtcttacacttccgcttacaaggttccattcgttgttatcgatttggtcaacttgagatccgttagagttaacgttgaaaaagctactgcttgggttgaatctggtgctactgttggtgaattatacgacgaaattgccagaaagtctaagaccttgggttttccagctggtatttgtccaactttaggtgttggtggtcacttgtctggtggtggttatggtttgttgtttagaaagtatggtttgggtgccgataacgtcattgatgctagattgattgatgtcaagggtcgtatcttggacaaaaagtctatgggtgaagatttgttttgggccattagaggtggtggtggcggtacttttggtatagttttggcttggaaattgaagttggttgctgttccagctaacgttactgttttcactgttagaaagactttggaacaaaacgccactaagttggttcatagatggcaatctattgcccacaagttcccaaaagaaatgcacttgtctattgctattgctagggctaactcttccgaagatgaagaaaagatgactatccaagcttctttcacctctactttcttgggttccattgatgaattgatcccattgatggaagaaaagttcccagaattgggtttagtcaaagaagattgcctagaaatgtcttgggctgagtctaacttgtacagctttttgttcttgctgagatctccatctgaaaccctgttgaatagaaaccagaagtcctctatctccaaaacctttttcaaggccaagtccgattttgtcaaacagccaattccagaatctgctttcgaaggtttatggtccaagttttatgaagatgaggctaagtccgccatcatggtttttgttgcttttggtggtaagatggacgaaatcttggaaactgaatacccatatccacatagagccggtaacttatacactatcttgtacactgttgactgggaagagaaagagaacatcaactctgaaaagttcatgtcctggatgagaagagtctacaattacatgactccctacgtttctaagtctccaagagaagcttacatcaactacagagatttggacatcggtacaaacaagatcaacaacagaaagagatcttacgctaaggctagagtatggggtgttaagtactttaagaacaacttcaacaggctggttaacgtcaagactatagttgacccacagaatttcttcagacacgaacaatctattccaccactattcttgtcctggaaaagatccgatcataagaagcacttgtcctcctggtcacatccacaatttgaaaag

SEQ ID NO: 105 - protein, 626 aa, synthetic construct
  REGION 1..626: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAMALPVQEFLHCLSLRSPNSSIAKLVYTQNNSSYSSVLKSAAQNFRFTTPSTPTPLAIVTPLHASHIQAAVYCCRKHGLQVRTRSGGHDFEGLSYTSAYKVPFVVIDLVNLRSVRVNVEKATAWVESGATVGELYDEIARKSKTLGFPAGICPTLGVGGHLSGGGYGLLFRKYGLGADNVIDARLIDVKGRILDKKSMGEDLFWAIRGGGGGTFGIVLAWKLKLVAVPANVTVFTVRKTLEQNATKLVHRWQSIAHKFPKEMHLSIAIARANSSEDEEKMTIQASFTSTFLGSIDELIPLMEEKFPELGLVKEDCLEMSWAESNLYSFLFLLRSPSETLLNRNQKSSISKTFFKAKSDFVKQPIPESAFEGLWSKFYEDEAKSAIMVFVAFGGKMDEILETEYPYPHRAGNLYTILYTVDWEEKENINSEKFMSWMRRVYNYMTPYVSKSPREAYINYRDLDIGTNKINNRKRSYAKARVWGVKYFKNNFNRLVNVKTIVDPQNFFRHEQSIPPLFLSWKRSDHKKHLSSWSHPQFEK

SEQ ID NO: 106 - DNA, 1638 bp, synthetic construct
  misc_feature 1..1638: note = CBS134-A0A1J3CT99
  source 1..1638: mol_type = other DNA
  CDS 1..1638
atgaggatctctaagccaattccagtcatctcctacattttgttcttggcccaatacttctcattgcacacaaaagaacctacctcttctacctcattgcaagatgatttcatcaagtgcttgcacagaaacaccaatgttgatttcccattggacaagactttcttcactccacaaagaaactcctccaacttcaaagaagttttgaacaccaccgtctggaatcaaagatatatgactaagtctatgcccaagccagagttcatttttaagccagttcatgaatcccatgtccaagcctctattatttgctctaagaagttgagaatccacttcagagttagatctggtggtcatgattatgagggtttgtcttacgtttctcaaatggaaactccattcgtcttgatcgatttgtggaagttgaagcaaatcaacgtcgacatcgaagataattctgcttgggttcaagctggtgctactactggtgagttgtattatagaattgctgccaagtctaaggttcatggttttccagctggtttgtgttctactgttggtataggtggtcatatcactggtggtggttatggtactttgatgagaaagtatggtttgtccaccgataacgttttggatgctagaatcgttgatgctaatggtcagttgttgaacagaactgctatgggtgaagatttgttctgggctattagaggtggtggtggcggttcatttggtgttattttggcttggaagatcaagttggttccagttccagaaactttgaccattttcactattaccaagaccttggaacaagatgccggtttcaagattttgtctaagtggcaaagaatcgtcgacaagttggtagaagaattgtccatcagagtcttgttcagaaacactggtaacaagactgttactactacctacagaggtgaattcttgggtgaaaaaggtacattgatggaagtcatgaagaagtgctttccagaattgggtttgactcaagaggattgcattgaaatgtcttggatcgaaactgttgtcaacaatgctggttttactactagaccacctccaccattggaaattttgttgcaaccatcttctccatacggtagaccatacttcaaagctaagtctgatttcgtcaaagaacccattccagttttgggtttaaagggtatgttcaagagaatgatcgaagaggatgcctctttcttgatttggaaaccatacggtggtatgatggctagaattccagaatccgaaattccatttccacacagaaacggtactatcttcaagatccattacttcaccaagtggtctgaaaacggttcttcttctgatgacaaacacatgaagtggatcagggaactgtattcttatatgaccccatacgtttcttctaacccaagacaagcttacgttaactacagagatttggacatcggtcagaacaagaagaatgactctgaagataactttagaagggctcaaatttggggtgctaagtactttaagaacaacttcaacaggctggtcaagatcaagacaaaagttgatccagaaaacttgttcaggcacgaacaatctattccaccattaccaattagaaggcagacctct

SEQ ID NO: 107 - protein, 546 aa, synthetic construct
  REGION 1..546: note = Synthetic Construct
MRISKPIPVISYILFLAQYFSLHTKEPTSSTSLQDDFIKCLHRNTNVDFPLDKTFFTPQRNSSNFKEVLNTTVWNQRYMTKSMPKPEFIFKPVHESHVQASIICSKKLRIHFRVRSGGHDYEGLSYVSQMETPFVLIDLWKLKQINVDIEDNSAWVQAGATTGELYYRIAAKSKVHGFPAGLCSTVGIGGHITGGGYGTLMRKYGLSTDNVLDARIVDANGQLLNRTAMGEDLFWAIRGGGGGSFGVILAWKIKLVPVPETLTIFTITKTLEQDAGFKILSKWQRIVDKLVEELSIRVLFRNTGNKTVTTTYRGEFLGEKGTLMEVMKKCFPELGLTQEDCIEMSWIETVVNNAGFTTRPPPPLEILLQPSSPYGRPYFKAKSDFVKEPIPVLGLKGMFKRMIEEDASFLIWKPYGGMMARIPESEIPFPHRNGTIFKIHYFTKWSENGSSSDDKHMKWIRELYSYMTPYVSSNPRQAYVNYRDLDIGQNKKNDSEDNFRRAQIWGAKYFKNNFNRLVKIKTKVDPENLFRHEQSIPPLPIRRQTS

SEQ ID NO: 108 - DNA, 1569 bp, synthetic construct
  misc_feature 1..1569: note = CBS134-A0A1J3CT99- no signal peptide
  source 1..1569: mol_type = other DNA
  CDS 1..1569
atgaaagaacctacctcttctacctcattgcaagatgatttcatcaagtgcttgcacagaaacaccaatgttgatttcccattggacaagactttcttcactccacaaagaaactcctccaacttcaaagaagttttgaacaccaccgtctggaatcaaagatatatgactaagtctatgcccaagccagagttcatttttaagccagttcatgaatcccatgtccaagcctctattatttgctctaagaagttgagaatccacttcagagttagatctggtggtcatgattatgagggtttgtcttacgtttctcaaatggaaactccattcgtcttgatcgatttgtggaagttgaagcaaatcaacgtcgacatcgaagataattctgcttgggttcaagctggtgctactactggtgagttgtattatagaattgctgccaagtctaaggttcatggttttccagctggtttgtgttctactgttggtataggtggtcatatcactggtggtggttatggtactttgatgagaaagtatggtttgtccaccgataacgttttggatgctagaatcgttgatgctaatggtcagttgttgaacagaactgctatgggtgaagatttgttctgggctattagaggtggtggtggcggttcatttggtgttattttggcttggaagatcaagttggttccagttccagaaactttgaccattttcactattaccaagaccttggaacaagatgccggtttcaagattttgtctaagtggcaaagaatcgtcgacaagttggtagaagaattgtccatcagagtcttgttcagaaacactggtaacaagactgttactactacctacagaggtgaattcttgggtgaaaaaggtacattgatggaagtcatgaagaagtgctttccagaattgggtttgactcaagaggattgcattgaaatgtcttggatcgaaactgttgtcaacaatgctggttttactactagaccacctccaccattggaaattttgttgcaaccatcttctccatacggtagaccatacttcaaagctaagtctgatttcgtcaaagaacccattccagttttgggtttaaagggtatgttcaagagaatgatcgaagaggatgcctctttcttgatttggaaaccatacggtggtatgatggctagaattccagaatccgaaattccatttccacacagaaacggtactatcttcaagatccattacttcaccaagtggtctgaaaacggttcttcttctgatgacaaacacatgaagtggatcagggaactgtattcttatatgaccccatacgtttcttctaacccaagacaagcttacgttaactacagagatttggacatcggtcagaacaagaagaatgactctgaagataactttagaagggctcaaatttggggtgctaagtactttaagaacaacttcaacaggctggtcaagatcaagacaaaagttgatccagaaaacttgttcaggcacgaacaatctattccaccattaccaattagaaggcagacctct

SEQ ID NO: 109 - protein, 523 aa, synthetic construct
  REGION 1..523: note = Synthetic Construct
MKEPTSSTSLQDDFIKCLHRNTNVDFPLDKTFFTPQRNSSNFKEVLNTTVWNQRYMTKSMPKPEFIFKPVHESHVQASIICSKKLRIHFRVRSGGHDYEGLSYVSQMETPFVLIDLWKLKQINVDIEDNSAWVQAGATTGELYYRIAAKSKVHGFPAGLCSTVGIGGHITGGGYGTLMRKYGLSTDNVLDARIVDANGQLLNRTAMGEDLFWAIRGGGGGSFGVILAWKIKLVPVPETLTIFTITKTLEQDAGFKILSKWQRIVDKLVEELSIRVLFRNTGNKTVTTTYRGEFLGEKGTLMEVMKKCFPELGLTQEDCIEMSWIETVVNNAGFTTRPPPPLEILLQPSSPYGRPYFKAKSDFVKEPIPVLGLKGMFKRMIEEDASFLIWKPYGGMMARIPESEIPFPHRNGTIFKIHYFTKWSENGSSSDDKHMKWIRELYSYMTPYVSSNPRQAYVNYRDLDIGQNKKNDSEDNFRRAQIWGAKYFKNNFNRLVKIKTKVDPENLFRHEQSIPPLPIRRQTS

SEQ ID NO: 110 - DNA, 1593 bp, synthetic construct
  misc_feature 1..1593: note = CBS134-A0A1J3CT99-no signal peptide-strep Tag
  source 1..1593: mol_type = other DNA
  CDS 1..1593
atgaaagaacctacctcttctacctcattgcaagatgatttcatcaagtgcttgcacagaaacaccaatgttgatttcccattggacaagactttcttcactccacaaagaaactcctccaacttcaaagaagttttgaacaccaccgtctggaatcaaagatatatgactaagtctatgcccaagccagagttcatttttaagccagttcatgaatcccatgtccaagcctctattatttgctctaagaagttgagaatccacttcagagttagatctggtggtcatgattatgagggtttgtcttacgtttctcaaatggaaactccattcgtcttgatcgatttgtggaagttgaagcaaatcaacgtcgacatcgaagataattctgcttgggttcaagctggtgctactactggtgagttgtattatagaattgctgccaagtctaaggttcatggttttccagctggtttgtgttctactgttggtataggtggtcatatcactggtggtggttatggtactttgatgagaaagtatggtttgtccaccgataacgttttggatgctagaatcgttgatgctaatggtcagttgttgaacagaactgctatgggtgaagatttgttctgggctattagaggtggtggtggcggttcatttggtgttattttggcttggaagatcaagttggttccagttccagaaactttgaccattttcactattaccaagaccttggaacaagatgccggtttcaagattttgtctaagtggcaaagaatcgtcgacaagttggtagaagaattgtccatcagagtcttgttcagaaacactggtaacaagactgttactactacctacagaggtgaattcttgggtgaaaaaggtacattgatggaagtcatgaagaagtgctttccagaattgggtttgactcaagaggattgcattgaaatgtcttggatcgaaactgttgtcaacaatgctggttttactactagaccacctccaccattggaaattttgttgcaaccatcttctccatacggtagaccatacttcaaagctaagtctgatttcgtcaaagaacccattccagttttgggtttaaagggtatgttcaagagaatgatcgaagaggatgcctctttcttgatttggaaaccatacggtggtatgatggctagaattccagaatccgaaattccatttccacacagaaacggtactatcttcaagatccattacttcaccaagtggtctgaaaacggttcttcttctgatgacaaacacatgaagtggatcagggaactgtattcttatatgaccccatacgtttcttctaacccaagacaagcttacgttaactacagagatttggacatcggtcagaacaagaagaatgactctgaagataactttagaagggctcaaatttggggtgctaagtactttaagaacaacttcaacaggctggtcaagatcaagacaaaagttgatccagaaaacttgttcaggcacgaacaatctattccaccattaccaattagaaggcagacctcttggtcacatccacaatttgaaaag

SEQ ID NO: 111 - protein, 531 aa, synthetic construct
  REGION 1..531: note = Synthetic Construct
MKEPTSSTSLQDDFIKCLHRNTNVDFPLDKTFFTPQRNSSNFKEVLNTTVWNQRYMTKSMPKPEFIFKPVHESHVQASIICSKKLRIHFRVRSGGHDYEGLSYVSQMETPFVLIDLWKLKQINVDIEDNSAWVQAGATTGELYYRIAAKSKVHGFPAGLCSTVGIGGHITGGGYGTLMRKYGLSTDNVLDARIVDANGQLLNRTAMGEDLFWAIRGGGGGSFGVILAWKIKLVPVPETLTIFTITKTLEQDAGFKILSKWQRIVDKLVEELSIRVLFRNTGNKTVTTTYRGEFLGEKGTLMEVMKKCFPELGLTQEDCIEMSWIETVVNNAGFTTRPPPPLEILLQPSSPYGRPYFKAKSDFVKEPIPVLGLKGMFKRMIEEDASFLIWKPYGGMMARIPESEIPFPHRNGTIFKIHYFTKWSENGSSSDDKHMKWIRELYSYMTPYVSSNPRQAYVNYRDLDIGQNKKNDSEDNFRRAQIWGAKYFKNNFNRLVKIKTKVDPENLFRHEQSIPPLPIRRQTSWSHPQFEK

SEQ ID NO: 112 - DNA, 1650 bp, synthetic construct
  misc_feature 1..1650: note = CBS134-A0A1J3CT99- truncated alpha factor - no signal peptide-strep Tag
  source 1..1650: mol_type = other DNA
  CDS 1..1650
atgagatttccttccatcttcacggctgtgctatttgcagcatcctccgcacttgcaatgaaagaacctacctcttctacctcattgcaagatgatttcatcaagtgcttgcacagaaacaccaatgttgatttcccattggacaagactttcttcactccacaaagaaactcctccaacttcaaagaagttttgaacaccaccgtctggaatcaaagatatatgactaagtctatgcccaagccagagttcatttttaagccagttcatgaatcccatgtccaagcctctattatttgctctaagaagttgagaatccacttcagagttagatctggtggtcatgattatgagggtttgtcttacgtttctcaaatggaaactccattcgtcttgatcgatttgtggaagttgaagcaaatcaacgtcgacatcgaagataattctgcttgggttcaagctggtgctactactggtgagttgtattatagaattgctgccaagtctaaggttcatggttttccagctggtttgtgttctactgttggtataggtggtcatatcactggtggtggttatggtactttgatgagaaagtatggtttgtccaccgataacgttttggatgctagaatcgttgatgctaatggtcagttgttgaacagaactgctatgggtgaagatttgttctgggctattagaggtggtggtggcggttcatttggtgttattttggcttggaagatcaagttggttccagttccagaaactttgaccattttcactattaccaagaccttggaacaagatgccggtttcaagattttgtctaagtggcaaagaatcgtcgacaagttggtagaagaattgtccatcagagtcttgttcagaaacactggtaacaagactgttactactacctacagaggtgaattcttgggtgaaaaaggtacattgatggaagtcatgaagaagtgctttccagaattgggtttgactcaagaggattgcattgaaatgtcttggatcgaaactgttgtcaacaatgctggttttactactagaccacctccaccattggaaattttgttgcaaccatcttctccatacggtagaccatacttcaaagctaagtctgatttcgtcaaagaacccattccagttttgggtttaaagggtatgttcaagagaatgatcgaagaggatgcctctttcttgatttggaaaccatacggtggtatgatggctagaattccagaatccgaaattccatttccacacagaaacggtactatcttcaagatccattacttcaccaagtggtctgaaaacggttcttcttctgatgacaaacacatgaagtggatcagggaactgtattcttatatgaccccatacgtttcttctaacccaagacaagcttacgttaactacagagatttggacatcggtcagaacaagaagaatgactctgaagataactttagaagggctcaaatttggggtgctaagtactttaagaacaacttcaacaggctggtcaagatcaagacaaaagttgatccagaaaacttgttcaggcacgaacaatctattccaccattaccaattagaaggcagacctcttggtcacatccacaatttgaaaag

SEQ ID NO: 113 - protein, 550 aa, synthetic construct
  REGION 1..550: note = Synthetic Construct
MRFPSIFTAVLFAASSALAMKEPTSSTSLQDDFIKCLHRNTNVDFPLDKTFFTPQRNSSNFKEVLNTTVWNQRYMTKSMPKPEFIFKPVHESHVQASIICSKKLRIHFRVRSGGHDYEGLSYVSQMETPFVLIDLWKLKQINVDIEDNSAWVQAGATTGELYYRIAAKSKVHGFPAGLCSTVGIGGHITGGGYGTLMRKYGLSTDNVLDARIVDANGQLLNRTAMGEDLFWAIRGGGGGSFGVILAWKIKLVPVPETLTIFTITKTLEQDAGFKILSKWQRIVDKLVEELSIRVLFRNTGNKTVTTTYRGEFLGEKGTLMEVMKKCFPELGLTQEDCIEMSWIETVVNNAGFTTRPPPPLEILLQPSSPYGRPYFKAKSDFVKEPIPVLGLKGMFKRMIEEDASFLIWKPYGGMMARIPESEIPFPHRNGTIFKIHYFTKWSENGSSSDDKHMKWIRELYSYMTPYVSSNPRQAYVNYRDLDIGQNKKNDSEDNFRRAQIWGAKYFKNNFNRLVKIKTKVDPENLFRHEQSIPPLPIRRQTSWSHPQFEK

SEQ ID NO: 114 - DNA, 1860 bp, synthetic construct
  misc_feature 1..1860: note = CBS134-A0A1J3CT99-full alpha factor-no signal peptide-strep Tag
  source 1..1860: mol_type = other DNA
  CDS 1..1860
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatgaaagaacctacctcttctacctcattgcaagatgatttcatcaagtgcttgcacagaaacaccaatgttgatttcccattggacaagactttcttcactccacaaagaaactcctccaacttcaaagaagttttgaacaccaccgtctggaatcaaagatatatgactaagtctatgcccaagccagagttcatttttaagccagttcatgaatcccatgtccaagcctctattatttgctctaagaagttgagaatccacttcagagttagatctggtggtcatgattatgagggtttgtcttacgtttctcaaatggaaactccattcgtcttgatcgatttgtggaagttgaagcaaatcaacgtcgacatcgaagataattctgcttgggttcaagctggtgctactactggtgagttgtattatagaattgctgccaagtctaaggttcatggttttccagctggtttgtgttctactgttggtataggtggtcatatcactggtggtggttatggtactttgatgagaaagtatggtttgtccaccgataacgttttggatgctagaatcgttgatgctaatggtcagttgttgaacagaactgctatgggtgaagatttgttctgggctattagaggtggtggtggcggttcatttggtgttattttggcttggaagatcaagttggttccagttccagaaactttgaccattttcactattaccaagaccttggaacaagatgccggtttcaagattttgtctaagtggcaaagaatcgtcgacaagttggtagaagaattgtccatcagagtcttgttcagaaacactggtaacaagactgttactactacctacagaggtgaattcttgggtgaaaaaggtacattgatggaagtcatgaagaagtgctttccagaattgggtttgactcaagaggattgcattgaaatgtcttggatcgaaactgttgtcaacaatgctggttttactactagaccacctccaccattggaaattttgttgcaaccatcttctccatacggtagaccatacttcaaagctaagtctgatttcgtcaaagaacccattccagttttgggtttaaagggtatgttcaagagaatgatcgaagaggatgcctctttcttgatttggaaaccatacggtggtatgatggctagaattccagaatccgaaattccatttccacacagaaacggtactatcttcaagatccattacttcaccaagtggtctgaaaacggttcttcttctgatgacaaacacatgaagtggatcagggaactgtattcttatatgaccccatacgtttcttctaacccaagacaagcttacgttaactacagagatttggacatcggtcagaacaagaagaatgactctgaagataactttagaagggctcaaatttggggtgctaagtactttaagaacaacttcaacaggctggtcaagatcaagacaaaagttgatccagaaaacttgttcaggcacgaacaatctattccaccattaccaattagaaggcagacctcttggtcacatccacaatttgaaaag

SEQ ID NO: 115 - protein, 620 aa, synthetic construct
  REGION 1..620: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAMKEPTSSTSLQDDFIKCLHRNTNVDFPLDKTFFTPQRNSSNFKEVLNTTVWNQRYMTKSMPKPEFIFKPVHESHVQASIICSKKLRIHFRVRSGGHDYEGLSYVSQMETPFVLIDLWKLKQINVDIEDNSAWVQAGATTGELYYRIAAKSKVHGFPAGLCSTVGIGGHITGGGYGTLMRKYGLSTDNVLDARIVDANGQLLNRTAMGEDLFWAIRGGGGGSFGVILAWKIKLVPVPETLTIFTITKTLEQDAGFKILSKWQRIVDKLVEELSIRVLFRNTGNKTVTTTYRGEFLGEKGTLMEVMKKCFPELGLTQEDCIEMSWIETVVNNAGFTTRPPPPLEILLQPSSPYGRPYFKAKSDFVKEPIPVLGLKGMFKRMIEEDASFLIWKPYGGMMARIPESEIPFPHRNGTIFKIHYFTKWSENGSSSDDKHMKWIRELYSYMTPYVSSNPRQAYVNYRDLDIGQNKKNDSEDNFRRAQIWGAKYFKNNFNRLVKIKTKVDPENLFRHEQSIPPLPIRRQTSWSHPQFEK

SEQ ID NO: 116 - DNA, 1638 bp, synthetic construct
  misc_feature 1..1638: note = CBS146-A0A1J3J6A2
  source 1..1638: mol_type = other DNA
  CDS 1..1638
atgaggatctctaagccaattccagtcatctcctacattttgttcttggcccaatacttctcattgcacacaaaagaacctacctcttctacctcattgcaagatgattttgttaagtgcttgcacagaaacaccaacgttgattttccattggacaagactttcttcaccccacaaagaaactcctccaacttcaaagaagtcttgaactctaccgtctggaatcaaagatatatgaccaagtctatgccaaagccagagttcatttttaagccagttcatgaatcccatgtccaagcctctattatttgctctaagaagttgagaatccacttcagagttagatctggtggtcatgattatgagggtttgtcttacgtttctcaaatggaaactccattcgtcttgatcgatttgtggaagttgaagcagattaacgtcgacatcgaagataattctgcttgggttcaagctggtgctactactggtgagttgtattatagaattgccgagaagtctaaggttcatggttttccagctggtttgtgttctactgttggtataggtggtcatatcactggtggtggttatggtactttgatgagaaagtatggtttgtccaccgataacgttttggatgctagaatcgttgatgctaatggtcagttgttgaacagaactgctatgggtgaagatttgttctgggctatttctggtggcggtggtggtagttttggtgttattttggcttggaagattaagttggttccagttccagaaaccttgaccattttcactattaccaagaccttggaacaagatgccggtttcaagattttgtccaaatggcaaagaatcgtcgacaagttggttgaagaattgtccatcagagtcttgttcagaaacactggtaacaagactgttactactacctacagaggtgaattcttgggtgaaaaaggtacattgatggaagtcatgaacaagtgctttccagaattgggtttgactcaagaggattgcattgaaatgtcttggatcgaaactgttgtcaacaatgctggttttactactagaccaccacctccattggaaattttgttgcaaccatcttctccatacggtagaccatacttcaaagctaagtctgatttcgtcaaagaacccattccagttttgggtttaaagggtatgttcaagagaatgatcgaagaggatgcctctttcttgatttggaaaccatacggtggtatgatggctagaattccagaatctgaattgccatttccacacagaaacggtactattttcaagatccattacttcaccaagtggtccgaaaatggttcttcttctgatgacaaacacatgaagtggatcagggaactgtattcttatatgaccccatacgtttcttctaacccaagacaagcttacgttaactacagagatttggacatcggtcagaacaagaagaacgattctgaaaacaatttcagaagggctcaaatttggggtgctaagtactttaagaacaacttcaacaggctggtcaagatcaagacaaaagttgatccagaaaacttgttcaggcacgaacaatctattccaccattgccaattagaaggcaaacctct

SEQ ID NO: 117 - protein, 546 aa, synthetic construct
  REGION 1..546: note = Synthetic Construct
MRISKPIPVISYILFLAQYFSLHTKEPTSSTSLQDDFVKCLHRNTNVDFPLDKTFFTPQRNSSNFKEVLNSTVWNQRYMTKSMPKPEFIFKPVHESHVQASIICSKKLRIHFRVRSGGHDYEGLSYVSQMETPFVLIDLWKLKQINVDIEDNSAWVQAGATTGELYYRIAEKSKVHGFPAGLCSTVGIGGHITGGGYGTLMRKYGLSTDNVLDARIVDANGQLLNRTAMGEDLFWAISGGGGGSFGVILAWKIKLVPVPETLTIFTITKTLEQDAGFKILSKWQRIVDKLVEELSIRVLFRNTGNKTVTTTYRGEFLGEKGTLMEVMNKCFPELGLTQEDCIEMSWIETVVNNAGFTTRPPPPLEILLQPSSPYGRPYFKAKSDFVKEPIPVLGLKGMFKRMIEEDASFLIWKPYGGMMARIPESELPFPHRNGTIFKIHYFTKWSENGSSSDDKHMKWIRELYSYMTPYVSSNPRQAYVNYRDLDIGQNKKNDSENNFRRAQIWGAKYFKNNFNRLVKIKTKVDPENLFRHEQSIPPLPIRRQTS

SEQ ID NO: 118 - DNA, 1569 bp, synthetic construct
  misc_feature 1..1569: note = CBS146-A0A1J3J6A2-no signal peptide
  source 1..1569: mol_type = other DNA
  CDS 1..1569
atgaaagaacctacctcttctacctcattgcaagatgattttgttaagtgcttgcacagaaacaccaacgttgattttccattggacaagactttcttcaccccacaaagaaactcctccaacttcaaagaagtcttgaactctaccgtctggaatcaaagatatatgaccaagtctatgccaaagccagagttcatttttaagccagttcatgaatcccatgtccaagcctctattatttgctctaagaagttgagaatccacttcagagttagatctggtggtcatgattatgagggtttgtcttacgtttctcaaatggaaactccattcgtcttgatcgatttgtggaagttgaagcagattaacgtcgacatcgaagataattctgcttgggttcaagctggtgctactactggtgagttgtattatagaattgccgagaagtctaaggttcatggttttccagctggtttgtgttctactgttggtataggtggtcatatcactggtggtggttatggtactttgatgagaaagtatggtttgtccaccgataacgttttggatgctagaatcgttgatgctaatggtcagttgttgaacagaactgctatgggtgaagatttgttctgggctatttctggtggcggtggtggtagttttggtgttattttggcttggaagattaagttggttccagttccagaaaccttgaccattttcactattaccaagaccttggaacaagatgccggtttcaagattttgtccaaatggcaaagaatcgtcgacaagttggttgaagaattgtccatcagagtcttgttcagaaacactggtaacaagactgttactactacctacagaggtgaattcttgggtgaaaaaggtacattgatggaagtcatgaacaagtgctttccagaattgggtttgactcaagaggattgcattgaaatgtcttggatcgaaactgttgtcaacaatgctggttttactactagaccaccacctccattggaaattttgttgcaaccatcttctccatacggtagaccatacttcaaagctaagtctgatttcgtcaaagaacccattccagttttgggtttaaagggtatgttcaagagaatgatcgaagaggatgcctctttcttgatttggaaaccatacggtggtatgatggctagaattccagaatctgaattgccatttccacacagaaacggtactattttcaagatccattacttcaccaagtggtccgaaaatggttcttcttctgatgacaaacacatgaagtggatcagggaactgtattcttatatgaccccatacgtttcttctaacccaagacaagcttacgttaactacagagatttggacatcggtcagaacaagaagaacgattctgaaaacaatttcagaagggctcaaatttggggtgctaagtactttaagaacaacttcaacaggctggtcaagatcaagacaaaagttgatccagaaaacttgttcaggcacgaacaatctattccaccattgccaattagaaggcaaacctct

SEQ ID NO: 119 - protein, 523 aa, synthetic construct
  REGION 1..523: note = Synthetic Construct
MKEPTSSTSLQDDFVKCLHRNTNVDFPLDKTFFTPQRNSSNFKEVLNSTVWNQRYMTKSMPKPEFIFKPVHESHVQASIICSKKLRIHFRVRSGGHDYEGLSYVSQMETPFVLIDLWKLKQINVDIEDNSAWVQAGATTGELYYRIAEKSKVHGFPAGLCSTVGIGGHITGGGYGTLMRKYGLSTDNVLDARIVDANGQLLNRTAMGEDLFWAISGGGGGSFGVILAWKIKLVPVPETLTIFTITKTLEQDAGFKILSKWQRIVDKLVEELSIRVLFRNTGNKTVTTTYRGEFLGEKGTLMEVMNKCFPELGLTQEDCIEMSWIETVVNNAGFTTRPPPPLEILLQPSSPYGRPYFKAKSDFVKEPIPVLGLKGMFKRMIEEDASFLIWKPYGGMMARIPESELPFPHRNGTIFKIHYFTKWSENGSSSDDKHMKWIRELYSYMTPYVSSNPRQAYVNYRDLDIGQNKKNDSENNFRRAQIWGAKYFKNNFNRLVKIKTKVDPENLFRHEQSIPPLPIRRQTS

SEQ ID NO: 120 - DNA, 1593 bp, synthetic construct
  misc_feature 1..1593: note = CBS146-A0A1J3J6A2- no signal peptide-strep Tag
  source 1..1593: mol_type = other DNA
  CDS 1..1593
atgaaagaacctacctcttctacctcattgcaagatgattttgttaagtgcttgcacagaaacaccaacgttgattttccattggacaagactttcttcaccccacaaagaaactcctccaacttcaaagaagtcttgaactctaccgtctggaatcaaagatatatgaccaagtctatgccaaagccagagttcatttttaagccagttcatgaatcccatgtccaagcctctattatttgctctaagaagttgagaatccacttcagagttagatctggtggtcatgattatgagggtttgtcttacgtttctcaaatggaaactccattcgtcttgatcgatttgtggaagttgaagcagattaacgtcgacatcgaagataattctgcttgggttcaagctggtgctactactggtgagttgtattatagaattgccgagaagtctaaggttcatggttttccagctggtttgtgttctactgttggtataggtggtcatatcactggtggtggttatggtactttgatgagaaagtatggtttgtccaccgataacgttttggatgctagaatcgttgatgctaatggtcagttgttgaacagaactgctatgggtgaagatttgttctgggctatttctggtggcggtggtggtagttttggtgttattttggcttggaagattaagttggttccagttccagaaaccttgaccattttcactattaccaagaccttggaacaagatgccggtttcaagattttgtccaaatggcaaagaatcgtcgacaagttggttgaagaattgtccatcagagtcttgttcagaaacactggtaacaagactgttactactacctacagaggtgaattcttgggtgaaaaaggtacattgatggaagtcatgaacaagtgctttccagaattgggtttgactcaagaggattgcattgaaatgtcttggatcgaaactgttgtcaacaatgctggttttactactagaccaccacctccattggaaattttgttgcaaccatcttctccatacggtagaccatacttcaaagctaagtctgatttcgtcaaagaacccattccagttttgggtttaaagggtatgttcaagagaatgatcgaagaggatgcctctttcttgatttggaaaccatacggtggtatgatggctagaattccagaatctgaattgccatttccacacagaaacggtactattttcaagatccattacttcaccaagtggtccgaaaatggttcttcttctgatgacaaacacatgaagtggatcagggaactgtattcttatatgaccccatacgtttcttctaacccaagacaagcttacgttaactacagagatttggacatcggtcagaacaagaagaacgattctgaaaacaatttcagaagggctcaaatttggggtgctaagtactttaagaacaacttcaacaggctggtcaagatcaagacaaaagttgatccagaaaacttgttcaggcacgaacaatctattccaccattgccaattagaaggcaaacctcttggtcacatccacaatttgaaaag

SEQ ID NO: 121 - protein, 531 aa, synthetic construct
  REGION 1..531: note = Synthetic Construct
MKEPTSSTSLQDDFVKCLHRNTNVDFPLDKTFFTPQRNSSNFKEVLNSTVWNQRYMTKSMPKPEFIFKPVHESHVQASIICSKKLRIHFRVRSGGHDYEGLSYVSQMETPFVLIDLWKLKQINVDIEDNSAWVQAGATTGELYYRIAEKSKVHGFPAGLCSTVGIGGHITGGGYGTLMRKYGLSTDNVLDARIVDANGQLLNRTAMGEDLFWAISGGGGGSFGVILAWKIKLVPVPETLTIFTITKTLEQDAGFKILSKWQRIVDKLVEELSIRVLFRNTGNKTVTTTYRGEFLGEKGTLMEVMNKCFPELGLTQEDCIEMSWIETVVNNAGFTTRPPPPLEILLQPSSPYGRPYFKAKSDFVKEPIPVLGLKGMFKRMIEEDASFLIWKPYGGMMARIPESELPFPHRNGTIFKIHYFTKWSENGSSSDDKHMKWIRELYSYMTPYVSSNPRQAYVNYRDLDIGQNKKNDSENNFRRAQIWGAKYFKNNFNRLVKIKTKVDPENLFRHEQSIPPLPIRRQTSWSHPQFEK

SEQ ID NO: 122 - DNA, 1650 bp, synthetic construct
  misc_feature 1..1650: note = CBS146-A0A1J3J6A2-truncated alpha factor - no signal peptide-strep Tag
  source 1..1650: mol_type = other DNA
  CDS 1..1650
atgagatttccttccatcttcacggctgtgctatttgcagcatcctccgcacttgcaatgaaagaacctacctcttctacctcattgcaagatgattttgttaagtgcttgcacagaaacaccaacgttgattttccattggacaagactttcttcaccccacaaagaaactcctccaacttcaaagaagtcttgaactctaccgtctggaatcaaagatatatgaccaagtctatgccaaagccagagttcatttttaagccagttcatgaatcccatgtccaagcctctattatttgctctaagaagttgagaatccacttcagagttagatctggtggtcatgattatgagggtttgtcttacgtttctcaaatggaaactccattcgtcttgatcgatttgtggaagttgaagcagattaacgtcgacatcgaagataattctgcttgggttcaagctggtgctactactggtgagttgtattatagaattgccgagaagtctaaggttcatggttttccagctggtttgtgttctactgttggtataggtggtcatatcactggtggtggttatggtactttgatgagaaagtatggtttgtccaccgataacgttttggatgctagaatcgttgatgctaatggtcagttgttgaacagaactgctatgggtgaagatttgttctgggctatttctggtggcggtggtggtagttttggtgttattttggcttggaagattaagttggttccagttccagaaaccttgaccattttcactattaccaagaccttggaacaagatgccggtttcaagattttgtccaaatggcaaagaatcgtcgacaagttggttgaagaattgtccatcagagtcttgttcagaaacactggtaacaagactgttactactacctacagaggtgaattcttgggtgaaaaaggtacattgatggaagtcatgaacaagtgctttccagaattgggtttgactcaagaggattgcattgaaatgtcttggatcgaaactgttgtcaacaatgctggttttactactagaccaccacctccattggaaattttgttgcaaccatcttctccatacggtagaccatacttcaaagctaagtctgatttcgtcaaagaacccattccagttttgggtttaaagggtatgttcaagagaatgatcgaagaggatgcctctttcttgatttggaaaccatacggtggtatgatggctagaattccagaatctgaattgccatttccacacagaaacggtactattttcaagatccattacttcaccaagtggtccgaaaatggttcttcttctgatgacaaacacatgaagtggatcagggaactgtattcttatatgaccccatacgtttcttctaacccaagacaagcttacgttaactacagagatttggacatcggtcagaacaagaagaacgattctgaaaacaatttcagaagggctcaaatttggggtgctaagtactttaagaacaacttcaacaggctggtcaagatcaagacaaaagttgatccagaaaacttgttcaggcacgaacaatctattccaccattgccaattagaaggcaaacctcttggtcacatccacaatttgaaaag

SEQ ID NO: 123 - protein, 550 aa, synthetic construct
  REGION 1..550: note = Synthetic Construct
MRFPSIFTAVLFAASSALAMKEPTSSTSLQDDFVKCLHRNTNVDFPLDKTFFTPQRNSSNFKEVLNSTVWNQRYMTKSMPKPEFIFKPVHESHVQASIICSKKLRIHFRVRSGGHDYEGLSYVSQMETPFVLIDLWKLKQINVDIEDNSAWVQAGATTGELYYRIAEKSKVHGFPAGLCSTVGIGGHITGGGYGTLMRKYGLSTDNVLDARIVDANGQLLNRTAMGEDLFWAISGGGGGSFGVILAWKIKLVPVPETLTIFTITKTLEQDAGFKILSKWQRIVDKLVEELSIRVLFRNTGNKTVTTTYRGEFLGEKGTLMEVMNKCFPELGLTQEDCIEMSWIETVVNNAGFTTRPPPPLEILLQPSSPYGRPYFKAKSDFVKEPIPVLGLKGMFKRMIEEDASFLIWKPYGGMMARIPESELPFPHRNGTIFKIHYFTKWSENGSSSDDKHMKWIRELYSYMTPYVSSNPRQAYVNYRDLDIGQNKKNDSENNFRRAQIWGAKYFKNNFNRLVKIKTKVDPENLFRHEQSIPPLPIRRQTSWSHPQFEK

SEQ ID NO: 124 - DNA, 1860 bp, synthetic construct
  misc_feature 1..1860: note = CBS146-A0A1J3J6A2- full alpha factor -no signal peptide-strep Tag
  source 1..1860: mol_type = other DNA
  CDS 1..1860
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatgaaagaacctacctcttctacctcattgcaagatgattttgttaagtgcttgcacagaaacaccaacgttgattttccattggacaagactttcttcaccccacaaagaaactcctccaacttcaaagaagtcttgaactctaccgtctggaatcaaagatatatgaccaagtctatgccaaagccagagttcatttttaagccagttcatgaatcccatgtccaagcctctattatttgctctaagaagttgagaatccacttcagagttagatctggtggtcatgattatgagggtttgtcttacgtttctcaaatggaaactccattcgtcttgatcgatttgtggaagttgaagcagattaacgtcgacatcgaagataattctgcttgggttcaagctggtgctactactggtgagttgtattatagaattgccgagaagtctaaggttcatggttttccagctggtttgtgttctactgttggtataggtggtcatatcactggtggtggttatggtactttgatgagaaagtatggtttgtccaccgataacgttttggatgctagaatcgttgatgctaatggtcagttgttgaacagaactgctatgggtgaagatttgttctgggctatttctggtggcggtggtggtagttttggtgttattttggcttggaagattaagttggttccagttccagaaaccttgaccattttcactattaccaagaccttggaacaagatgccggtttcaagattttgtccaaatggcaaagaatcgtcgacaagttggttgaagaattgtccatcagagtcttgttcagaaacactggtaacaagactgttactactacctacagaggtgaattcttgggtgaaaaaggtacattgatggaagtcatgaacaagtgctttccagaattgggtttgactcaagaggattgcattgaaatgtcttggatcgaaactgttgtcaacaatgctggttttactactagaccaccacctccattggaaattttgttgcaaccatcttctccatacggtagaccatacttcaaagctaagtctgatttcgtcaaagaacccattccagttttgggtttaaagggtatgttcaagagaatgatcgaagaggatgcctctttcttgatttggaaaccatacggtggtatgatggctagaattccagaatctgaattgccatttccacacagaaacggtactattttcaagatccattacttcaccaagtggtccgaaaatggttcttcttctgatgacaaacacatgaagtggatcagggaactgtattcttatatgaccccatacgtttcttctaacccaagacaagcttacgttaactacagagatttggacatcggtcagaacaagaagaacgattctgaaaacaatttcagaagggctcaaatttggggtgctaagtactttaagaacaacttcaacaggctggtcaagatcaagacaaaagttgatccagaaaacttgttcaggcacgaacaatctattccaccattgccaattagaaggcaaacctcttggtcacatccacaatttgaaaag

SEQ ID NO: 125 - protein, 620 aa, synthetic construct
  REGION 1..620: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAMKEPTSSTSLQDDFVKCLHRNTNVDFPLDKTFFTPQRNSSNFKEVLNSTVWNQRYMTKSMPKPEFIFKPVHESHVQASIICSKKLRIHFRVRSGGHDYEGLSYVSQMETPFVLIDLWKLKQINVDIEDNSAWVQAGATTGELYYRIAEKSKVHGFPAGLCSTVGIGGHITGGGYGTLMRKYGLSTDNVLDARIVDANGQLLNRTAMGEDLFWAISGGGGGSFGVILAWKIKLVPVPETLTIFTITKTLEQDAGFKILSKWQRIVDKLVEELSIRVLFRNTGNKTVTTTYRGEFLGEKGTLMEVMNKCFPELGLTQEDCIEMSWIETVVNNAGFTTRPPPPLEILLQPSSPYGRPYFKAKSDFVKEPIPVLGLKGMFKRMIEEDASFLIWKPYGGMMARIPESELPFPHRNGTIFKIHYFTKWSENGSSSDDKHMKWIRELYSYMTPYVSSNPRQAYVNYRDLDIGQNKKNDSENNFRRAQIWGAKYFKNNFNRLVKIKTKVDPENLFRHEQSIPPLPIRRQTSWSHPQFEK

SEQ ID NO: 126 - DNA, 1629 bp, synthetic construct
  misc_feature 1..1629: note = CBS152-A0A1S3BE31
  source 1..1629: mol_type = other DNA
  CDS 1..1629
atgaagtccttgatcacctctttcttgcacatcttcatcgttttgtcttttgcttgggctgcttctgctcataatcatgaatctttcttacactgcttgtcccatcattctccaaacacctcttctatctccaagattatctacactccaactaacccctcttactcctctgttttgaacttctccattcacaacttgagattcacctctccaaaaactccaaagccacatattatcgttaccccattcttggtgtctcaaattcaagcctctattatctgcgctaaaaagaccggtttccaaatcagaactagatctggtggtcatgattacgaaggtttgtcttacgtttccgatgtttccttcgttgttgttgacttgattaacctgagatccatttctgttgatgccgaaaacaatgttgcttgggttcaatctggtgctactattggtgagttgtactacagaattgccgaaaagtctaagaccttgggttttccagctggtgtttgtccaactgttggtgttggtggtcacttttctggtggtggttatggtttgatgttgagaaaatttggtttggccgctgataacgtcattgatgcttacttcattgatgtcaacggtaagctgcatgacagaaaatctatgggtgaagatgttttctgggctattagaggtggtggtggcgcttcttttggtatagttttggcttggaagatcaagttgttgccagttccatctatcgttactgttttcactattaccagaaccttggaacaaaacgccaccgatttgattcatagatggcaatacgtttcttctaagcaggatgacaagttgttcatcagaattatcttgaccggtatcaacaccaccaattcacaacatggtaacaagagaactattgaagctgccttcaactctttgttcttgggtaagattgaagagttggtcccaattatggaaaagactttcccagaattgggtttgaccaaagaagattgcattgagatgtcctggattgagtctgtcttgtactttgctggtttttctagaggtcaaccattgaacgttttgttggatagaaggccattgactccaaagagatttttcaaggctaagtccgattacgtcaacgaacctattccaaaagctggtttggaaggtatttgggagttgttcgatgaagaagaagctgaagctgctgttttgattttgtctccatacggtggtatcatggacaagatttccgaatccgaaattccatttccacatagagctggcaacctgtacaaaatccaacatttggtttactgggacgaagaaggtgaagatattgctaagagacatatcaactggatcagaaagctgtactcttacatggctccattcgtttctaaaaatccaagagctgcctacatcaactacagagatttggatattggcaccaacgaaaagaacggtagaaacacttcttacaacgaagcttctgtttggggtgttaagtactttaagggtaacttcaagaggctggtttctgttaagacaaaggttgacccatccaatttcttcaagaacgaacaatccattccatccttgaaaccatggtggaacaagagaggttat

SEQ ID NO: 127 - protein, 543 aa, synthetic construct
  REGION 1..543: note = Synthetic Construct
MKSLITSFLHIFIVLSFAWAASAHNHESFLHCLSHHSPNTSSISKIIYTPTNPSYSSVLNFSIHNLRFTSPKTPKPHIIVTPFLVSQIQASIICAKKTGFQIRTRSGGHDYEGLSYVSDVSFVVVDLINLRSISVDAENNVAWVQSGATIGELYYRIAEKSKTLGFPAGVCPTVGVGGHFSGGGYGLMLRKFGLAADNVIDAYFIDVNGKLHDRKSMGEDVFWAIRGGGGASFGIVLAWKIKLLPVPSIVTVFTITRTLEQNATDLIHRWQYVSSKQDDKLFIRIILTGINTTNSQHGNKRTIEAAFNSLFLGKIEELVPIMEKTFPELGLTKEDCIEMSWIESVLYFAGFSRGQPLNVLLDRRPLTPKRFFKAKSDYVNEPIPKAGLEGIWELFDEEEAEAAVLILSPYGGIMDKISESEIPFPHRAGNLYKIQHLVYWDEEGEDIAKRHINWIRKLYSYMAPFVSKNPRAAYINYRDLDIGTNEKNGRNTSYNEASVWGVKYFKGNFKRLVSVKTKVDPSNFFKNEQSIPSLKPWWNKRGY

SEQ ID NO: 128 - DNA, 1566 bp, synthetic construct
  misc_feature 1..1566: note = CBS152-A0A1S3BE31- no signal peptide
  source 1..1566: mol_type = other DNA
  CDS 1..1566
atggctcataatcatgaatctttcttacactgcttgtcccatcattctccaaacacctcttctatctccaagattatctacactccaactaacccctcttactcctctgttttgaacttctccattcacaacttgagattcacctctccaaaaactccaaagccacatattatcgttaccccattcttggtgtctcaaattcaagcctctattatctgcgctaaaaagaccggtttccaaatcagaactagatctggtggtcatgattacgaaggtttgtcttacgtttccgatgtttccttcgttgttgttgacttgattaacctgagatccatttctgttgatgccgaaaacaatgttgcttgggttcaatctggtgctactattggtgagttgtactacagaattgccgaaaagtctaagaccttgggttttccagctggtgtttgtccaactgttggtgttggtggtcacttttctggtggtggttatggtttgatgttgagaaaatttggtttggccgctgataacgtcattgatgcttacttcattgatgtcaacggtaagctgcatgacagaaaatctatgggtgaagatgttttctgggctattagaggtggtggtggcgcttcttttggtatagttttggcttggaagatcaagttgttgccagttccatctatcgttactgttttcactattaccagaaccttggaacaaaacgccaccgatttgattcatagatggcaatacgtttcttctaagcaggatgacaagttgttcatcagaattatcttgaccggtatcaacaccaccaattcacaacatggtaacaagagaactattgaagctgccttcaactctttgttcttgggtaagattgaagagttggtcccaattatggaaaagactttcccagaattgggtttgaccaaagaagattgcattgagatgtcctggattgagtctgtcttgtactttgctggtttttctagaggtcaaccattgaacgttttgttggatagaaggccattgactccaaagagatttttcaaggctaagtccgattacgtcaacgaacctattccaaaagctggtttggaaggtatttgggagttgttcgatgaagaagaagctgaagctgctgttttgattttgtctccatacggtggtatcatggacaagatttccgaatccgaaattccatttccacatagagctggcaacctgtacaaaatccaacatttggtttactgggacgaagaaggtgaagatattgctaagagacatatcaactggatcagaaagctgtactcttacatggctccattcgtttctaaaaatccaagagctgcctacatcaactacagagatttggatattggcaccaacgaaaagaacggtagaaacacttcttacaacgaagcttctgtttggggtgttaagtactttaagggtaacttcaagaggctggtttctgttaagacaaaggttgacccatccaatttcttcaagaacgaacaatccattccatccttgaaaccatggtggaacaagagaggttat

SEQ ID NO: 129 - protein, 522 aa, synthetic construct
  REGION 1..522: note = Synthetic Construct
MAHNHESFLHCLSHHSPNTSSISKIIYTPTNPSYSSVLNFSIHNLRFTSPKTPKPHIIVTPFLVSQIQASIICAKKTGFQIRTRSGGHDYEGLSYVSDVSFVVVDLINLRSISVDAENNVAWVQSGATIGELYYRIAEKSKTLGFPAGVCPTVGVGGHFSGGGYGLMLRKFGLAADNVIDAYFIDVNGKLHDRKSMGEDVFWAIRGGGGASFGIVLAWKIKLLPVPSIVTVFTITRTLEQNATDLIHRWQYVSSKQDDKLFIRIILTGINTTNSQHGNKRTIEAAFNSLFLGKIEELVPIMEKTFPELGLTKEDCIEMSWIESVLYFAGFSRGQPLNVLLDRRPLTPKRFFKAKSDYVNEPIPKAGLEGIWELFDEEEAEAAVLILSPYGGIMDKISESEIPFPHRAGNLYKIQHLVYWDEEGEDIAKRHINWIRKLYSYMAPFVSKNPRAAYINYRDLDIGTNEKNGRNTSYNEASVWGVKYFKGNFKRLVSVKTKVDPSNFFKNEQSIPSLKPWWNKRGY

SEQ ID NO: 130 - DNA, 1590 bp, synthetic construct
  misc_feature 1..1590: note = CBS152-A0A1S3BE31- no signal peptide-strep Tag
  source 1..1590: mol_type = other DNA
  CDS 1..1590
atggctcataatcatgaatctttcttacactgcttgtcccatcattctccaaacacctcttctatctccaagattatctacactccaactaacccctcttactcctctgttttgaacttctccattcacaacttgagattcacctctccaaaaactccaaagccacatattatcgttaccccattcttggtgtctcaaattcaagcctctattatctgcgctaaaaagaccggtttccaaatcagaactagatctggtggtcatgattacgaaggtttgtcttacgtttccgatgtttccttcgttgttgttgacttgattaacctgagatccatttctgttgatgccgaaaacaatgttgcttgggttcaatctggtgctactattggtgagttgtactacagaattgccgaaaagtctaagaccttgggttttccagctggtgtttgtccaactgttggtgttggtggtcacttttctggtggtggttatggtttgatgttgagaaaatttggtttggccgctgataacgtcattgatgcttacttcattgatgtcaacggtaagctgcatgacagaaaatctatgggtgaagatgttttctgggctattagaggtggtggtggcgcttcttttggtatagttttggcttggaagatcaagttgttgccagttccatctatcgttactgttttcactattaccagaaccttggaacaaaacgccaccgatttgattcatagatggcaatacgtttcttctaagcaggatgacaagttgttcatcagaattatcttgaccggtatcaacaccaccaattcacaacatggtaacaagagaactattgaagctgccttcaactctttgttcttgggtaagattgaagagttggtcccaattatggaaaagactttcccagaattgggtttgaccaaagaagattgcattgagatgtcctggattgagtctgtcttgtactttgctggtttttctagaggtcaaccattgaacgttttgttggatagaaggccattgactccaaagagatttttcaaggctaagtccgattacgtcaacgaacctattccaaaagctggtttggaaggtatttgggagttgttcgatgaagaagaagctgaagctgctgttttgattttgtctccatacggtggtatcatggacaagatttccgaatccgaaattccatttccacatagagctggcaacctgtacaaaatccaacatttggtttactgggacgaagaaggtgaagatattgctaagagacatatcaactggatcagaaagctgtactcttacatggctccattcgtttctaaaaatccaagagctgcctacatcaactacagagatttggatattggcaccaacgaaaagaacggtagaaacacttcttacaacgaagcttctgtttggggtgttaagtactttaagggtaacttcaagaggctggtttctgttaagacaaaggttgacccatccaatttcttcaagaacgaacaatccattccatccttgaaaccatggtggaacaagagaggttattggtcacatccacaatttgaaaag

SEQ ID NO: 131 - protein, 530 aa, synthetic construct
  REGION 1..530: note = Synthetic Construct
MAHNHESFLHCLSHHSPNTSSISKIIYTPTNPSYSSVLNFSIHNLRFTSPKTPKPHIIVTPFLVSQIQASIICAKKTGFQIRTRSGGHDYEGLSYVSDVSFVVVDLINLRSISVDAENNVAWVQSGATIGELYYRIAEKSKTLGFPAGVCPTVGVGGHFSGGGYGLMLRKFGLAADNVIDAYFIDVNGKLHDRKSMGEDVFWAIRGGGGASFGIVLAWKIKLLPVPSIVTVFTITRTLEQNATDLIHRWQYVSSKQDDKLFIRIILTGINTTNSQHGNKRTIEAAFNSLFLGKIEELVPIMEKTFPELGLTKEDCIEMSWIESVLYFAGFSRGQPLNVLLDRRPLTPKRFFKAKSDYVNEPIPKAGLEGIWELFDEEEAEAAVLILSPYGGIMDKISESEIPFPHRAGNLYKIQHLVYWDEEGEDIAKRHINWIRKLYSYMAPFVSKNPRAAYINYRDLDIGTNEKNGRNTSYNEASVWGVKYFKGNFKRLVSVKTKVDPSNFFKNEQSIPSLKPWWNKRGYWSHPQFEK

SEQ ID NO: 132 - DNA, 1647 bp, synthetic construct
  misc_feature 1..1647: note = CBS152-A0A1S3BE31- no signal peptide-truncated alpha factor-strep Tag
  source 1..1647: mol_type = other DNA
  CDS 1..1647
atgagatttccttccatcttcacggctgtgctatttgcagcatcctccgcacttgcaatggctcataatcatgaatctttcttacactgcttgtcccatcattctccaaacacctcttctatctccaagattatctacactccaactaacccctcttactcctctgttttgaacttctccattcacaacttgagattcacctctccaaaaactccaaagccacatattatcgttaccccattcttggtgtctcaaattcaagcctctattatctgcgctaaaaagaccggtttccaaatcagaactagatctggtggtcatgattacgaaggtttgtcttacgtttccgatgtttccttcgttgttgttgacttgattaacctgagatccatttctgttgatgccgaaaacaatgttgcttgggttcaatctggtgctactattggtgagttgtactacagaattgccgaaaagtctaagaccttgggttttccagctggtgtttgtccaactgttggtgttggtggtcacttttctggtggtggttatggtttgatgttgagaaaatttggtttggccgctgataacgtcattgatgcttacttcattgatgtcaacggtaagctgcatgacagaaaatctatgggtgaagatgttttctgggctattagaggtggtggtggcgcttcttttggtatagttttggcttggaagatcaagttgttgccagttccatctatcgttactgttttcactattaccagaaccttggaacaaaacgccaccgatttgattcatagatggcaatacgtttcttctaagcaggatgacaagttgttcatcagaattatcttgaccggtatcaacaccaccaattcacaacatggtaacaagagaactattgaagctgccttcaactctttgttcttgggtaagattgaagagttggtcccaattatggaaaagactttcccagaattgggtttgaccaaagaagattgcattgagatgtcctggattgagtctgtcttgtactttgctggtttttctagaggtcaaccattgaacgttttgttggatagaaggccattgactccaaagagatttttcaaggctaagtccgattacgtcaacgaacctattccaaaagctggtttggaaggtatttgggagttgttcgatgaagaagaagctgaagctgctgttttgattttgtctccatacggtggtatcatggacaagatttccgaatccgaaattccatttccacatagagctggcaacctgtacaaaatccaacatttggtttactgggacgaagaaggtgaagatattgctaagagacatatcaactggatcagaaagctgtactcttacatggctccattcgtttctaaaaatccaagagctgcctacatcaactacagagatttggatattggcaccaacgaaaagaacggtagaaacacttcttacaacgaagcttctgtttggggtgttaagtactttaagggtaacttcaagaggctggtttctgttaagacaaaggttgacccatccaatttcttcaagaacgaacaatccattccatccttgaaaccatggtggaacaagagaggttattggtcacatccacaatttgaaaag

SEQ ID NO: 133 - protein, 549 aa, synthetic construct
  REGION 1..549: note = Synthetic Construct
MRFPSIFTAVLFAASSALAMAHNHESFLHCLSHHSPNTSSISKIIYTPTNPSYSSVLNFSIHNLRFTSPKTPKPHIIVTPFLVSQIQASIICAKKTGFQIRTRSGGHDYEGLSYVSDVSFVVVDLINLRSISVDAENNVAWVQSGATIGELYYRIAEKSKTLGFPAGVCPTVGVGGHFSGGGYGLMLRKFGLAADNVIDAYFIDVNGKLHDRKSMGEDVFWAIRGGGGASFGIVLAWKIKLLPVPSIVTVFTITRTLEQNATDLIHRWQYVSSKQDDKLFIRIILTGINTTNSQHGNKRTIEAAFNSLFLGKIEELVPIMEKTFPELGLTKEDCIEMSWIESVLYFAGFSRGQPLNVLLDRRPLTPKRFFKAKSDYVNEPIPKAGLEGIWELFDEEEAEAAVLILSPYGGIMDKISESEIPFPHRAGNLYKIQHLVYWDEEGEDIAKRHINWIRKLYSYMAPFVSKNPRAAYINYRDLDIGTNEKNGRNTSYNEASVWGVKYFKGNFKRLVSVKTKVDPSNFFKNEQSIPSLKPWWNKRGYWSHPQFEK

SEQ ID NO: 134 - DNA, 1857 bp, synthetic construct
  misc_feature 1..1857: note = CBS152-A0A1S3BE31- no signal peptide-full alpha factor -strep Tag
  source 1..1857: mol_type = other DNA
  CDS 1..1857
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatggctcataatcatgaatctttcttacactgcttgtcccatcattctccaaacacctcttctatctccaagattatctacactccaactaacccctcttactcctctgttttgaacttctccattcacaacttgagattcacctctccaaaaactccaaagccacatattatcgttaccccattcttggtgtctcaaattcaagcctctattatctgcgctaaaaagaccggtttccaaatcagaactagatctggtggtcatgattacgaaggtttgtcttacgtttccgatgtttccttcgttgttgttgacttgattaacctgagatccatttctgttgatgccgaaaacaatgttgcttgggttcaatctggtgctactattggtgagttgtactacagaattgccgaaaagtctaagaccttgggttttccagctggtgtttgtccaactgttggtgttggtggtcacttttctggtggtggttatggtttgatgttgagaaaatttggtttggccgctgataacgtcattgatgcttacttcattgatgtcaacggtaagctgcatgacagaaaatctatgggtgaagatgttttctgggctattagaggtggtggtggcgcttcttttggtatagttttggcttggaagatcaagttgttgccagttccatctatcgttactgttttcactattaccagaaccttggaacaaaacgccaccgatttgattcatagatggcaatacgtttcttctaagcaggatgacaagttgttcatcagaattatcttgaccggtatcaacaccaccaattcacaacatggtaacaagagaactattgaagctgccttcaactctttgttcttgggtaagattgaagagttggtcccaattatggaaaagactttcccagaattgggtttgaccaaagaagattgcattgagatgtcctggattgagtctgtcttgtactttgctggtttttctagaggtcaaccattgaacgttttgttggatagaaggccattgactccaaagagatttttcaaggctaagtccgattacgtcaacgaacctattccaaaagctggtttggaaggtatttgggagttgttcgatgaagaagaagctgaagctgctgttttgattttgtctccatacggtggtatcatggacaagatttccgaatccgaaattccatttccacatagagctggcaacctgtacaaaatccaacatttggtttactgggacgaagaaggtgaagatattgctaagagacatatcaactggatcagaaagctgtactcttacatggctccattcgtttctaaaaatccaagagctgcctacatcaactacagagatttggatattggcaccaacgaaaagaacggtagaaacacttcttacaacgaagcttctgtttggggtgttaagtactttaagggtaacttcaagaggctggtttctgttaagacaaaggttgacccatccaatttcttcaagaacgaacaatccattccatccttgaaaccatggtggaacaagagaggttattggtcacatccacaatttgaaaag

SEQ ID NO: 135 - protein, 619 aa, synthetic construct
  REGION 1..619: note = Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAMAHNHESFLHCLSHHSPNTSSISKIIYTPTNPSYSSVLNFSIHNLRFTSPKTPKPHIIVTPFLVSQIQASIICAKKTGFQIRTRSGGHDYEGLSYVSDVSFVVVDLINLRSISVDAENNVAWVQSGATIGELYYRIAEKSKTLGFPAGVCPTVGVGGHFSGGGYGLMLRKFGLAADNVIDAYFIDVNGKLHDRKSMGEDVFWAIRGGGGASFGIVLAWKIKLLPVPSIVTVFTITRTLEQNATDLIHRWQYVSSKQDDKLFIRIILTGINTTNSQHGNKRTIEAAFNSLFLGKIEELVPIMEKTFPELGLTKEDCIEMSWIESVLYFAGFSRGQPLNVLLDRRPLTPKRFFKAKSDYVNEPIPKAGLEGIWELFDEEEAEAAVLILSPYGGIMDKISESEIPFPHRAGNLYKIQHLVYWDEEGEDIAKRHINWIRKLYSYMAPFVSKNPRAAYINYRDLDIGTNEKNGRNTSYNEASVWGVKYFKGNFKRLVSVKTKVDPSNFFKNEQSIPSLKPWWNKRGYWSHPQFEK